SEQUENCE LISTING (68 sequences)

SEQ ID NO: 1 - DNA, 1263 bp, Artificial Sequence
  Her-2 chimeric protein
gagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagactaa

SEQ ID NO: 2 - protein, 420 aa, Artificial Sequence [sequence omitted]

SEQ ID NO: 3 - DNA, 1323 bp, Listeria monocytogenes
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgat

SEQ ID NO: 4 - protein, 441 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 5 - protein, 14 aa, Listeria monocytogenes
KTEEQPSEVNTGPR

SEQ ID NO: 6 - protein, 28 aa, Listeria monocytogenes
KASVTDTSEGDLDSSMQSADESTPQPLK

SEQ ID NO: 7 - protein, 20 aa, Listeria monocytogenes
KNEEVNASDFPPPPTDEELR

SEQ ID NO: 8 - protein, 33 aa, Listeria monocytogenes
RGGIPTSEEFSSLNSGDFTDDENSETTEEEIDR

SEQ ID NO: 9 - protein, 17 aa, Streptococcus pyogenes
KQNTASTETTTTNEQPK

SEQ ID NO: 10 - protein, 17 aa, Streptococcus equisimilis
KQNTANTETTTTNEQPK

SEQ ID NO: 11 - protein, 9 aa, Homo sapiens
HLYQGCQVV

SEQ ID NO: 12 - protein, 9 aa, Homo sapiens
KIFGSLAFL

SEQ ID NO: 13 - protein, 9 aa, Homo sapiens
RLLQETELV

SEQ ID NO: 14 - protein, 55 aa, Mus musculus
GGTCACAGCTGAGGACGGAACACAGCGTTGTGAGAAATGCAGCAAGCCCTGTGCT

SEQ ID NO: 15 - protein, 60 aa, Mus musculus
CGAGTGTGCTATGGTCTGGGCATGGAGCACCTTCGAGGGGCGAGGGCCATCACCAGTGAC

SEQ ID NO: 16 - protein, 60 aa, Mus musculus
AATGTCCAGGAGTTTGATGGCTGCAAGAAGATCTTTGGGAGCCTGGCATTTTTGCCGGAG

SEQ ID NO: 17 - protein, 60 aa, Mus musculus
AGCTTTGATGGGGACCCCTCCTCCGGCATTGCTCCGCTGAGGCCTGAGCAGCTCCAAGTG

SEQ ID NO: 18 - protein, 60 aa, Mus musculus
TTCGAAACCCTGGAGGAGATCACAGGTTACCTGTACATCTCAGCATGGCCAGACAGTCTC

SEQ ID NO: 19 - protein, 60 aa, Mus musculus
CGTGACCTCAGTGTCTTCCAGAACCTTCGAATCATTCGGGGACGGATTCTCCACGATGGC

SEQ ID NO: 20 - protein, 60 aa, Mus musculus
GCGTACTCATTGACACTGCAAGGCCTGGGGATCCACTCGCTGGGGCTGCGCTCACTGCGG

SEQ ID NO: 21 - protein, 60 aa, Mus musculus
GAGCTGGGCAGTGGATTGGCTCTGATTCACCGCAACGCCCATCTCTGCTTTGTACACACT

SEQ ID NO: 22 - protein, 60 aa, Mus musculus
GTACCTTGGGACCAGCTCTTCCGGAACCCACATCAGGCCCTGCTCCACAGTGGGAACCGG

SEQ ID NO: 23 - protein, 60 aa, Mus musculus
CCGGAAGAGGATTGTGGTCTCGAGGGCTTGGTCTGTAACTCACTGTGTGCCCACGGGCAC

SEQ ID NO: 24 - protein, 60 aa, Mus musculus
TGCTGGGGGCCAGGGCCCACCCAGTGTGTCAACTGCAGTCATTTCCTTCGGGGCCAGGAG

SEQ ID NO: 25 - protein, 57 aa, Mus musculus
CGCCCAGCGGAGCAATGCCCAACCAGGCTCAGATGCGGATCCTAAAAGAGACGGAGC

SEQ ID NO: 26 - protein, 60 aa, Mus musculus
TAAGGAAGGTGAAGGTGCTTGGATCAGGAGCTTTTGGCACTGTCTACAAGGGCATCTGGA

SEQ ID NO: 27 - protein, 60 aa, Mus musculus
TCCCAGATGGGGAGAATGTGAAAATCCCCGTGGCTATCAAGGTGTTGAGAGAAAACACAT

SEQ ID NO: 28 - protein, 60 aa, Mus musculus
CTCCTAAAGCCAACAAAGAAATTCTAGATGAAGCGTATGTGATGGCTGGTGTGGGTTCTC

SEQ ID NO: 29 - protein, 60 aa, Mus musculus
CGTATGTGTCCCGCCTCCTGGGCATCTGCCTGACATCCACAGTACAGCTGGTGACACAGC

SEQ ID NO: 30 - protein, 60 aa, Mus musculus
TTATGCCCTACGGCTGCCTTCTGGACCATGTCCGAGAACACCGAGGTCGCCTAGGCTCCC

SEQ ID NO: 31 - protein, 60 aa, Mus musculus
AGGACCTGCTCAACTGGTGTGTTCAGATTGCCAAGGGGATGAGCTACCTGGAGGACGTGC

SEQ ID NO: 32 - protein, 60 aa, Mus musculus
GGCTTGTACACAGGGACCTGGCTGCCCGGAATGTGCTAGTCAAGAGTCCCAACCACGTCA

SEQ ID NO: 33 - protein, 60 aa, Mus musculus
AGATTACAGATTTCGGGCTGGCTCGGCTGCTGGACATTGATGAGACAGAGTACCATGCAG

SEQ ID NO: 34 - protein, 60 aa, Mus musculus
ATGGGGGCAAGGTGCCCATCAAATGGATGGCATTGGAATCTATTCTCAGACGCCGGTTCA

SEQ ID NO: 35 - protein, 60 aa, Mus musculus
CCCATCAGAGTGATGTGTGGAGCTATGGAGTGACTGTGTGGGAGCTGATGACTTTTGGGG

SEQ ID NO: 36 - protein, 59 aa, Mus musculus
CCAAACCTTACGATGGAATCCCAGCCCGGGAGATCCCTGATTTGCTGGAGAAGGGAGAA

SEQ ID NO: 37 - protein, 58 aa, Mus musculus
CGCCTACCTCAGCCTCCAATCTGCACCATTGATGTCTACATGATTATGGTCAAATGTT

SEQ ID NO: 38 - protein, 54 aa, Mus musculus
GGATGATTGACTCTGAATGTCGCCCGAGATTCCGGGAGTTGGTGTCAGAATTTT

SEQ ID NO: 39 - protein, 52 aa, Mus musculus
CACGTATGGCGAGGGACCCCCAGCGTTTTGTGGTCATCCAGAACGAGGACTT

SEQ ID NO: 40 - protein, 60 aa, Mus musculus
CCCAGGCAGAACCCCAGAGGGGCTGCGGGAGCTGCAGCTTCGAAGTCTCACAGAGATCCT

SEQ ID NO: 41 - protein, 60 aa, Mus musculus
GAAGGGAGGAGTTTTGATCCGTGGGAACCCTCAGCTCTGCTACCAGGACATGGTTTTGTG

SEQ ID NO: 42 - protein, 60 aa, Mus musculus
CCGGGCCTGTCCACCTTGTGCCCCCGCCTGCAAAGACAATCACTGTTGGGGTGAGAGTCC

SEQ ID NO: 43 - protein, 60 aa, Mus musculus
GGAAGACTGTCAGATCTTGACTGGCACCATCTGTACCAGTGGTTGTGCCCGGTGCAAGGG

SEQ ID NO: 44 - protein, 60 aa, Mus musculus
CCGGCTGCCCACTGACTGCTGCCATGAGCAGTGTGCCGCAGGCTGCACGGGCCCCAAGCA

SEQ ID NO: 45 - DNA, 3716 bp, Rattus norvegicus
ccggaatcgcgggcacccaagtgtgtaccggcacagacatgaagttgcggctccctgccagtcctgagacccacctggacatgctccgccacctgtaccagggctgtcaggtagtgcagggcaacttggagcttacctacgtgcctgccaatgccagcctctcattcctgcaggacatccaggaagttcagggttacatgctcatcgctcacaaccaggtgaagcgcgtcccactgcaaaggctgcgcatcgtgagagggacccagctctttgaggacaagtatgccctggctgtgctagacaaccgagatcctcaggacaatgtcgccgcctccaccccaggcagaaccccagaggggctgcgggagctgcagcttcgaagtctcacagagatcctgaagggaggagttttgatccgtgggaaccctcagctctgctaccaggacatggttttgtggaaggacgtcttccgcaagaataaccaactggctcctgtcgatatagacaccaatcgttcccgggcctgtccaccttgtgcccccgcctgcaaagacaatcactgttggggtgagagtccggaagactgtcagatcttgactggcaccatctgtaccagtggttgtgcccggtgcaagggccggctgcccactgactgctgccatgagcagtgtgccgcaggctgcacgggccccaagcattctgactgcctggcctgcctccacttcaatcatagtggtatctgtgagctgcactgcccagccctcgtcacctacaacacagacacctttgagtccatgcacaaccctgagggtcgctacacctttggtgccagctgcgtgaccacctgcccctacaactacctgtctacggaagtgggatcctgcactctggtgtgtcccccgaataaccaagaggtcacagctgaggacggaacacagcgttgtgagaaatgcagcaagccctgtgctcgagtgtgctatggtctgggcatggagcaccttcgaggggcgagggccatcaccagtgacaatgtccaggagtttgatggctgcaagaagatctttgggagcctggcatttttgccggagagctttgatggggacccctcctccggcattgctccgctgaggcctgagcagctccaagtgttcgaaaccctggaggagatcacaggttacctgtacatctcagcatggccagacagtctccgtgacctcagtgtcttccagaaccttcgaatcattcggggacggattctccacgatggcgcgtactcattgacactgcaaggcctggggatccactcgctggggctgcgctcactgcgggagctgggcagtggattggctctgattcaccgcaacgcccatctctgctttgtacacactgtaccttgggaccagctcttccggaacccacatcaggccctgctccacagtgggaaccggccggaagaggattgtggtctcgagggcttggtctgtaactcactgtgtgcccacgggcactgctgggggccagggcccacccagtgtgtcaactgcagtcatttccttcggggccaggagtgtgtggaggagtgccgagtatggaaggggctcccccgggagtatgtgagtgacaagcgctgtctgccgtgtcaccccgagtgtcagcctcaaaacagctcagagacctgctttggatcggaggctgatcagtgtgcagcctgcgcccactacaaggactcgtcctcctgtgtggctcgctgccccagtggtgtgaaaccggacctctcctacatgcccatctggaagtacccggatgaggagggcatatgccagccgtgccccatcaactgcacccactcctgtgtggatctggatgaacgaggctgcccagcagagcagagagccagcccggtgacattcatcattgcaactgtagtgggcgtcctgctgttcctgatcttagtggtggtcgttggaatcctaatcaaacgaaggagacagaagatccggaagtatacgatgcgtaggctgctgcaggaaactgagttagtggagccgctgacgcccagcggagcaatgcccaaccaggctcagatgcggatcctaaaagagacggagctaaggaaggtgaaggtgcttggatcaggagcttttggcactgtctacaagggcatctggatcccagatggggagaatgtgaaaatccccgtggctatcaaggtgttgagagaaaacacatctcctaaagccaacaaagaaattctagatgaagcgtatgtgatggctggtgtgggttctccgtatgtgtcccgcctcctgggcatctgcctgacatccacagtacagctggtgacacagcttatgccctacggctgccttctggaccatgtccgagaacaccgaggtcgcctaggctcccaggacctgctcaactggtgtgttcagattgccaaggggatgagctacctggaggacgtgcggcttgtacacagggacctggctgcccggaatgtgctagtcaagagtcccaaccacgtcaagattacagatttcgggctggctcggctgctggacattgatgagacagagtaccatgcagatgggggcaaggtgcccatcaaatggatggcattggaatctattctcagacgccggttcacccatcagagtgatgtgtggagctatggagtgactgtgtgggagctgatgacttttggggccaaaccttacgatggaatcccagcccgggagatccctgatttgctggagaagggagaacgcctacctcagcctccaatctgcaccattgatgtctacatgattatggtcaaatgttggatgattgactctgaatgtcgcccgagattccgggagttggtgtcagaattttcacgtatggcgagggacccccagcgttttgtggtcatccagaacgaggacttgggcccatccagccccatggacagtaccttctaccgttcactgctggaagatgatgacatgggtgacctggtagacgctgaagagtatctggtgccccagcagggattcttctccccggaccctaccccaggcactgggagcacagcccatagaaggcaccgcagctcgtccaccaggagtggaggtggtgagctgacactgggcctggagccctcggaagaagggccccccagatctccactggctccctcggaaggggctggctccgatgtgtttgatggtgacctggcaatgggggtaaccaaagggctgcagagcctctctccacatgacctcagccctctacagcggtacagcgaggaccccacattacctctgccccccgagactgatggctatgttgctcccctggcctgcagcccccagcccgagtatgtgaaccaatcagaggttcagcctcagcctcctttaaccccagagggtcctctgcctcctgtccggcctgctggtgctactctagaaagacccaagactctctctcctgggaagaatggggttgtcaaagacgtttttgccttcgggggtgctgtggagaaccctgaatacttagtaccgagagaaggcactgcctctccgccccacccttctcctgccttcagcccagcctttgacaacctctattactgggaccagaactcatcggagcaggggcctccaccaagtaactttgaagggacccccactgcagagaaccctgagtacctaggcctggatgtacctgta

SEQ ID NO: 46 - DNA, 360 bp, Rattus norvegicus
cccaggcagaaccccagaggggctgcgggagctgcagcttcgaagtctcacagagatcctgaagggaggagttttgatccgtgggaaccctcagctctgctaccaggacatggttttgtggaaggacgtcttccgcaagaataaccaactggctcctgtcgatatagacaccaatcgttcccgggcctgtccaccttgtgcccccgcctgcaaagacaatcactgttggggtgagagtccggaagactgtcagatcttgactggcaccatctgtaccagtggttgtgcccggtgcaagggccggctgcccactgactgctgccatgagcagtgtgccgcaggctgcacgggccccaagca

SEQ ID NO: 47 - DNA, 618 bp, Rattus norvegicus
ggtcacagctgaggacggaacacagcgttgtgagaaatgcagcaagccctgtgctcgagtgtgctatggtctgggcatggagcaccttcgaggggcgagggccatcaccagtgacaatgtccaggagtttgatggctgcaagaagatctttgggagcctggcatttttgccggagagctttgatggggacccctcctccggcattgctccgctgaggcctgagcagctccaagtgttcgaaaccctggaggagatcacaggttacctgtacatctcagcatggccagacagtctccgtgacctcagtgtcttccagaaccttcgaatcattcggggacggattctccacgatggcgcgtactcattgacactgcaaggcctggggatccactcgctggggctgcgctcactgcgggagctgggcagtggattggctctgattcaccgcaacgcccatctctgctttgtacacactgtaccttgggaccagctcttccggaacccacatcaggccctgctccacagtgggaaccggccggaagaggattgtggtctcgagggcttggtctgtaactcactgtgtgcccacgggcactgctgggggccagggcccaccca

SEQ ID NO: 48 - DNA, 929 bp, Rattus norvegicus
cgcccagcggagcaatgcccaaccaggctcagatgcggatcctaaaagagacggagctaaggaaggtgaaggtgcttggatcaggagcttttggcactgtctacaagggcatctggatcccagatggggagaatgtgaaaatccccgtggctatcaaggtgttgagagaaaacacatctcctaaagccaacaaagaaattctagatgaagcgtatgtgatggctggtgtgggttctccgtatgtgtcccgcctcctgggcatctgcctgacatccacagtacagctggtgacacagcttatgccctacggctgccttctggaccatgtccgagaacaccgaggtcgcctaggctcccaggacctgctcaactggtgtgttcagattgccaaggggatgagctacctggaggacgtgcggcttgtacacagggacctggctgcccggaatgtgctagtcaagagtcccaaccacgtcaagattacagatttcgggctggctcggctgctggacattgatgagacagagtaccatgcagatgggggcaaggtgcccatcaaatggatggcattggaatctattctcagacgccggttcacccatcagagtgatgtgtggagctatggagtgactgtgtgggagctgatgacttttggggccaaaccttacgatggaatcccagcccgggagatccctgatttgctggagaagggagaacgcctacctcagcctccaatctgcaccattgatgtctacatgattatggtcaaatgttggatgattgactctgaatgtcgcccgagattccgggagttggtgtcagaattttcacgtatggcgagggacccccagcgttttgtggtcatccagaacgaggacttgggcccatccagccccatggacagtaccttctaccgttcactgctggaa

SEQ ID NO: 49 - DNA, 3798 bp, Homo sapiens
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctga

SEQ ID NO: 50 - DNA, 393 bp, Homo sapiens
gagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaag

SEQ ID NO: 51 - protein, 477 aa, Homo sapiens
AATATCCAGGAGTTTGCTGGCTGCAAGAAGATCTTTGGGAGCCTGGCATTTCTGCCGGAGAGCTTTGATGGGGACCCAGCCTCCAACACTGCCCCGCTCCAGCCAGAGCAGCTCCAAGTGTTTGAGACTCTGGAAGAGATCACAGGTTACCTATACATCTCAGCATGGCCGGACAGCCTGCCTGACCTCAGCGTCTTCCAGAACCTGCAAGTAATCCGGGGACGAATTCTGCACAATGGCGCCTACTCGCTGACCCTGCAAGGGCTGGGCATCAGCTGGCTGGGGCTGCGCTCACTGAGGGAACTGGGCAGTGGACTGGCCCTCATCCACCATAACACCCACCTCTGCTTCGTGCACACGGTGCCCTGGGACCAGCTCTTTCGGAACCCGCACCAAGCTCTGCTCCACACTGCCAACCGGCCAGAGGACGAGTGTGTGGGCGAGGGCCTGGCCTGCCACCAGCTGTGCGCCCGAGGG

SEQ ID NO: 52 - DNA, 391 bp, Homo sapiens
cagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagact

SEQ ID NO: 53 - DNA, 7075 bp, Artificial Sequence
  Plasmid sequence of pAdv164
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagactaatctagacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 54 - DNA, 921 bp, Homo sapiens
gccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggat

SEQ ID NO: 55 - DNA, 597 bp, Homo sapiens
tacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagag

SEQ ID NO: 56 - DNA, 1209 bp, Homo sapiens
cagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggct

SEQ ID NO: 57 - DNA, 28 bp, Artificial Sequence
  Her-2-Chimera (F)
tgatctcgagacccacctggacatgctc

SEQ ID NO: 58 - DNA, 49 bp, Artificial Sequence
  HerEC1-EC2F (Junction)
ctaccaggacacgattttgtggaagaatatccaggagtttgctggctgc

SEQ ID NO: 59 - DNA, 49 bp, Artificial Sequence
  HerEC1-EC2R (Junction)
gcagccagcaaactcctggatattcttccacaaaatcgtgtcctggtag

SEQ ID NO: 60 - DNA, 50 bp, Artificial Sequence
  HerEC2-ICIF (Junction)
ctgccaccagctgtgcgcccgagggcagcagaagatccggaagtacacga

SEQ ID NO: 61 - DNA, 50 bp, Artificial Sequence
  HerEC2-ICIR (Junction)
tcgtgtacttccggatcttctgctgccctcgggcgcacagctggtggcag

SEQ ID NO: 62 - DNA, 39 bp, Artificial Sequence
  Her-2-Chimera (R)
gtggcccgggtctagattagtctaagaggcagccatagg

SEQ ID NO: 63 - DNA, 28 bp, Artificial Sequence
  Her-2-EC1(F)
ccgcctcgaggccgcgagcacccaagtg

SEQ ID NO: 64 - DNA, 31 bp, Artificial Sequence
  Her-2-EC1(R)
cgcgactagtttaatcctctgctgtcacctc

SEQ ID NO: 65 - DNA, 28 bp, Artificial Sequence
  Her-2-EC2(F)
ccgcctcgagtacctttctacggacgtg

SEQ ID NO: 66 - DNA, 30 bp, Artificial Sequence
  Her- 2- EC2(R)
cgcgactagtttactctggccggttggcag

SEQ ID NO: 67 - DNA, 31 bp, Artificial Sequence
  Her-2-Her-2-IC1(F)
ccgcctcgagcagcagaagatccggaagtac

SEQ ID NO: 68 - DNA, 30 bp, Artificial Sequence
  Her-2-IC1(R)
cgcgactagtttaagccccttcggagggtg

(19) United States
(12) Patent Application Publication
(10) Pub. No.: US 2014/0234370 A1
(43) Pub. Date: Aug. 21, 2014

(54) COMPOSITIONS AND METHODS FOR PREVENTION OF ESCAPE MUTATION IN THE TREATMENT OF HER2/NEU OVER-EXPRESSING TUMORS

(71) Applicant: Advaxis, Inc., Princeton, NJ (US)

(72) Inventor: Vafa SHAHABI, Valley Forge, PA (US)

(73) Assignee: Advaxis, Inc., Princeton, NJ (US)

(21) Appl. No.: 14/268,436

(22) Filed: May 2, 2014

Related U.S. Application Data
(63) Continuation-in-part of application No. 14/189,008, filed on Feb. 25, 2014.
Application No. 14/189,008 is a continuation-in-part of application No. 13/210,696, filed on Aug. 16, 2011.
Application No. 13/210,696 is a continuation-in-part of application No. 12/945,386, filed on Nov. 12, 2010.
(60) Provisional application No. 61/260,277, filed on Nov. 11, 2009.

(51) Int. Cl. A61K 39/02; A61K 35/74

(52) U.S. Cl. CPC A61K 39/0208; A61K 35/74; A61K 2039/585
USPC 424/200.1; 424/93.2

(57) ABSTRACT

This invention provides compositions and methods for treating and vaccinating against a Her2/neu antigen-expressing tumor and inducing an immune response against dominant in a human subject.

### CROSS-REFERENCE TO RELATED APPLICATIONS

This application is a Continuation-In-Part of co-pending U.S. patent application Ser. No. 14/189,008, filed on Feb. 25, 2014, which is a Continuation-In-Part of U.S. patent application Ser. No. 13/210,696 filed on Aug. 16, 2011, which is a Continuation-In-Part of U.S. patent application Ser. No. 12/945,386, filed Nov. 12, 2010, which claims the benefit of U.S. Provisional Application Ser. No. 61/260,277, filed Nov. 11, 2009. These applications are hereby incorporated in their entirety by reference herein.

### FIELD OF INVENTION

This invention provides compositions and methods for inducing an immune response against a Her2/neu antigen-expressing tumor and for treating the same and vaccinating against the same in a human subject, wherein the human subject is a child or adolescent.

### BACKGROUND OF THE INVENTION

Her-2/neu (referred to henceforth as “Her-2”) is a 185 kDa glycoprotein that is a member of the epidermal growth factor receptor (EGFR) family of tyrosine kinases, and consists of an extracellular domain, a transmembrane domain, and an intracellular domain which is known to be involved in cellular signaling. In humans, the Her2 antigen is overexpressed in 25 to 40% of all breast cancers and is also overexpressed in many cancers of the ovaries, lung, pancreas, brain, and gastrointestinal tract. The overexpression of Her-2 is associated with uncontrolled cell growth and signaling, both of which contribute to the development of tumors. Patients with cancers that overexpress Her-2 exhibit tolerance even with detectable humoral, CD8⁺ T cell, and CD4⁺ T cell responses directed against Her-2.

Listeria monocytogenes is an intracellular pathogen that primarily infects antigen presenting cells and has adapted for life in the cytoplasm of these cells. Host cells, such as macrophages, actively phagocytose L. monocytogenes and the majority of the bacteria are degraded in the phagolysosome. Some of the bacteria escape into the host cytosol by perforating the phagosomal membrane through the action of a hemolysin, listeriolysin O (LLO). Once in the cytosol, L. monocytogenes can polymerize the host actin and pass directly from cell to cell further evading the host immune system and resulting in a negligible antibody response to L. monocytogenes.

Primary malignant bone tumors in the pediatric to young adult populations are relatively uncommon and account for about 6% of all cancers in those less than 20 years old and 3% of all cancers in adolescents and young adults (AYA) within the age range of 15 to 29 years. Osteosarcoma affects about 400 children and teens in the U.S. every year, representing a small, high need area that has seen little therapeutics improvement in decades. Although osteosarcoma (OS) is a rare malignancy, it is ranked among the leading causes of cancer-related death in the pediatric age group. Modern, multiagent, dose-intensive chemotherapy in conjunction with surgery achieves a 5-year event-free survival of 60-70% in extremity localized, non-metastatic disease. However, a major, as yet unsolved, problem is the poor prognosis for metastatic relapse or recurrence, and for patients with axial disease. Moreover, there are no products approved for osteosarcoma in the U.S, presenting a high need for novel therapies that address this disease.

The present invention meets this need by providing a recombinant Listeria-Her2/neu vaccine (ADXS31-164) that was generated using the LmddA vaccine vector which has a well-defined attenuation mechanism and is devoid of antibiotic selection markers and which has been found effective in treating canine osteosarcoma.

### SUMMARY OF THE INVENTION

In one embodiment, the invention provided herein relates to an immunogenic composition comprising a fusion polypeptide, wherein said fusion polypeptide comprises a Her2/neu chimeric antigen fused to an additional polypeptide, and wherein administering the fusion protein to a subject having a Her2/neu-expressing tumor invokes mutation avoidance. In another embodiment, mutation avoidance is due to epitope spreading. In yet another embodiment, mutation avoidance is due to the chimeric nature of the antigen.

In another embodiment, the invention provided herein relates to a recombinant Listeria vaccine strain comprising a nucleic acid molecule, wherein and in another embodiment, the nucleic acid molecule comprises a first open reading frame encoding a polypeptide, wherein the polypeptide comprises a Her2/neu chimeric antigen, wherein the nucleic acid molecule further comprises a second open reading frame encoding a metabolic enzyme, and wherein the metabolic enzyme complements an endogenous gene that is lacking in the chromosome of the recombinant Listeria strain.

In one embodiment, the invention provided herein relates to a method of treating a Her-2/neu-expressing tumor growth or cancer in a human subject, the method comprising the step of administering a recombinant Listeria comprising nucleic acid encoding a fusion polypeptide, wherein said fusion polypeptide comprises a Her2/neu chimeric antigen fused to an additional adjuvant polypeptide.

In another embodiment, the invention provided herein relates to a method of preventing a Her-2/neu-expressing tumor growth or cancer in a human subject, the method comprising the step of administering a recombinant Listeria comprising nucleic acid encoding a fusion polypeptide, wherein said fusion polypeptide comprises a Her2/neu chimeric antigen fused to an additional adjuvant polypeptide.

In one embodiment, the invention provided herein relates to a method of eliciting an enhanced immune response against a Her-2/neu-expressing tumor growth or cancer in a human subject, the method comprising the step of administering a recombinant Listeria comprising a nucleic encoding a fusion polypeptide, wherein said fusion polypeptide comprises a Her2/neu chimeric antigen fused to an additional adjuvant polypeptide.

### BRIEF DESCRIPTION OF THE FIGURES

FIG. 1. Construction of ADXS31-164. (A) Plasmid map of pAdv164, which harbors bacillus subtilis dal gene under the control of constitutive Listeria p60 promoter for complementation of the chromosomal dal-dat deletion in LmddA strain. It also contains the fusion of truncated LLO₍₁₋₄₄₁₎ to the chimeric human Her2/neu gene, which was constructed by the direct fusion of 3 fragments the Her2/neu: EC1 (aa 40-170), EC2 (aa 359-518) and ICI (aa 679-808). (B) Expression and secretion of tLLO-ChHer2 was detected in Lm-LLO-ChHer2 (Lm-LLO-138) and LmddA-LLO-ChHer2 (ADXS31-164) by western blot analysis of the TCA precipitated cell culture supernatants blotted with anti-LLO antibody. A differential band of ˜104 KD corresponds to tLLO-ChHer2. The endogenous LLO is detected as a 58 KD band. Listeria control lacked ChHer2 expression.

FIG. 2. Immunogenic properties of ADXS31-164 (A) Cytotoxic T cell responses elicited by Her2/neu Listeria-based vaccines in splenocytes from immunized mice were tested using NT-2 cells as stimulators and 3T3/neu cells as targets. Lm-control was based on the LmddA background that was identical in all ways but expressed an irrelevant antigen (HPV16-E7). (B) IFN-γ secreted by the splenocytes from immunized FVB/N mice into the cell culture medium, measured by ELISA, after 24 hours of in vitro stimulation with mitomycin C treated NT-2 cells. (C) IFN-γ secretion by splenocytes from HLA-A2 transgenic mice immunized with the chimeric vaccine, in response to in vitro incubation with peptides from different regions of the protein. A recombinant ChHer2 protein was used as positive control and an irrelevant peptide or no peptide groups constituted the negative controls as listed in the figure legend. IFN-γ secretion was detected by an ELISA assay using cell culture supernatants harvested after 72 hours of co-incubation. Each data point was an average of triplicate data+/−standard error. * P value<0.001.

FIG. 3. Tumor Prevention Studies for Listeria-ChHer2/neu Vaccines Her2/neu transgenic mice were injected six times with each recombinant Listeria-ChHer2 or a control Listeria vaccine Immunizations started at 6 weeks of age and continued every three weeks until week 21. Appearance of tumors was monitored on a weekly basis and expressed as percentage of tumor free mice. *p<0.05, N=9 per group.

FIG. 4. Effect of immunization with ADXS31-164 on the % of Tregs in Spleens. FVB/N mice were inoculated s.c. with 1×10⁶ NT-2 cells and immunized three times with each vaccine at one week intervals. Spleens were harvested 7 days after the second immunization. After isolation of the immune cells, they were stained for detection of Tregs by anti CD3, CD4, CD25 and FoxP3 antibodies. dot-plots of the Tregs from a representative experiment showing the frequency of CD25⁺/FoxP3⁺ T cells, expressed as percentages of the total CD3⁺ or CD3⁺CD4⁺ T cells across the different treatment groups.

FIG. 5. Effect of immunization with ADXS31-164 on the % of tumor infiltrating Tregs in NT-2 tumors. FVB/N mice were inoculated s.c. with 1×10⁶ NT-2 cells and immunized three times with each vaccine at one week intervals. Tumors were harvested 7 days after the second immunization. After isolation of the immune cells, they were stained for detection of Tregs by anti CD3, CD4, CD25 and FoxP3 antibodies. (A). dot-plots of the Tregs from a representative experiment. (B). Frequency of CD25⁺/FoxP3⁺ T cells, expressed as percentages of the total CD3⁺ or CD3⁺CD4⁺ T cells (left panel) and intratumoral CD8/Tregs ratio (right panel) across the different treatment groups. Data is shown as mean±SEM obtained from 2 independent experiments.

FIG. 6. Vaccination with ADXS31-164 can delay the growth of a breast cancer cell line in the brain. Balb/c mice were immunized thrice with ADXS31-164 or a control Listeria vaccine. EMT6-Luc cells (5,000) were injected intracranially in anesthetized mice. (A) Ex vivo imaging of the mice was performed on the indicated days using a Xenogen X-100 CCD camera. (B) Pixel intensity was graphed as number of photons per second per cm2 of surface area; this is shown as average radiance. (C) Expression of Her2/neu by EMT6-Luc cells, 4T1-Luc and NT-2 cell lines was detected by Western blots, using an anti-Her2/neu antibody. J774.A2 cells, a murine macrophage like cell line was used as a negative control.

FIG. 7. Shows the first 18 patients vaccinated with ADXS31-164.

FIG. 8. Shows intravenous administration of ADXS31-164 causes mild transient fever with no systemic hypotension.

FIG. 9. Shows that ADXS31-164 administration does not cause early or late cardiac damage.

FIG. 10. Shows the evaluation of combination ADXS31-164 and palliative radiation therapy (RT) in primary disease.

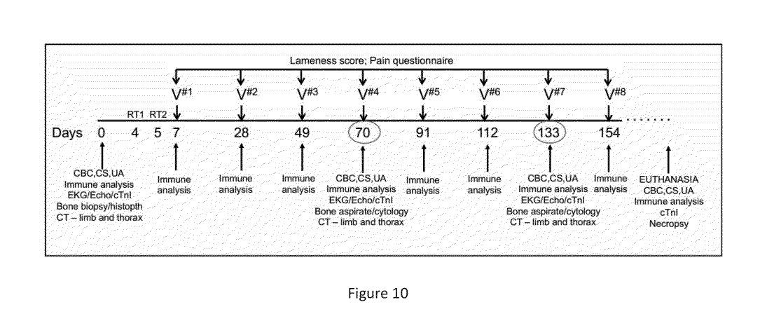

FIG. 11. Shows no evidence of metastatic disease following fracture of proximal humerus and also shows the presence of boney callus indicating fracture healing.

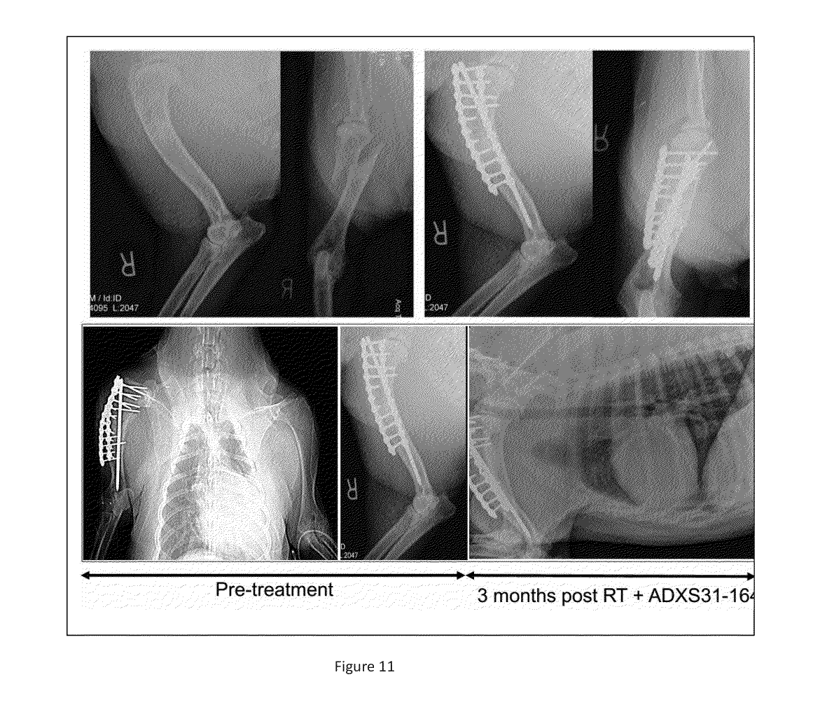

FIG. 12. Timeline of a pilot phase I clinical trial to evaluate the safety and efficacy of a L. monocytogenes recombinant expressing huHer2/neu to elicit therapeutically effective anti-tumor immunity in dogs with appendicular osteosarcoma.

FIG. 13. Treatment-related adverse events and survival curves following ADXS-31-164 administration. A) Treatment-related adverse events. B) All dogs that completed vaccination treatment. Dogs in the control group underwent limb amputation followed by either carboplatin alone of carboplatin plus Adriamycin. 4 dogs have been censored from the vaccine arm: 3 dogs had metastatic disease at the time of vaccination and came off trial after first vaccine (2 dogs) and second vaccine (1 dog) due to progressive metastatic disease. One dog died from a disease unrelated to OSA.C) All dogs that enrolled in the trial.

### DETAILED DESCRIPTION OF THE INVENTION

In one embodiment, provided herein are compositions and methods for preventing, treating and vaccinating against a Her2-neu antigen-expressing tumor and inducing an immune response against sub-dominant epitopes of the Her2-neu antigen, while invoking mutation avoidance. In another embodiment, mutation avoidance is due to epitope spreading. In yet another embodiment, mutation avoidance is due to the chimeric nature of the antigen.

In another embodiment, provided herein is an immunogenic composition comprising a fusion polypeptide, wherein said fusion polypeptide comprises a Her2/neu chimeric antigen fused to an additional polypeptide, and wherein administering the fusion protein to a subject having an Her2/neu-expressing tumor prevents escape mutations within said tumor. In another embodiment, provided herein is a recombinant Listeria vaccine strain comprising the immunogenic composition.

In one embodiment, the subject is a canine. In another embodiment, the canine is a dog.

In one embodiment, provided herein is a method of eliciting an enhanced immune response against a Her-2/neu-expressing tumor growth or cancer in a human subject, the method comprising the step of administering a recombinant Listeria comprising a nucleic encoding a fusion polypeptide, wherein said fusion polypeptide comprises a Her2/neu chimeric antigen fused to an additional adjuvant polypeptide.

In another embodiment, provided herein is a method of preventing a Her-2/neu-expressing tumor growth or cancer in a human subject, the method comprising the step of administering a recombinant Listeria comprising nucleic acid encoding a fusion polypeptide, wherein said fusion polypeptide comprises a Her2/neu chimeric antigen fused to an additional adjuvant polypeptide.

In one embodiment, provided herein is a method of treating a Her-2/neu-expressing tumor growth or cancer in a human subject, the method comprising the step of administering a recombinant Listeria comprising nucleic acid encoding a fusion polypeptide, wherein said fusion polypeptide comprises a Her2/neu chimeric antigen fused to an additional adjuvant polypeptide. In another embodiment, the human subject is a canine. In yet another embodiment, the canine is a dog.

In one embodiment, provided herein is a recombinant Listeria vaccine strain comprising a nucleic acid molecule, wherein the nucleic acid molecule comprises a first open reading frame encoding a polypeptide, wherein the polypeptide comprises a Her2/neu chimeric antigen, wherein the nucleic acid molecule further comprises a second open reading frame encoding a metabolic enzyme, and wherein the metabolic enzyme complements an endogenous gene that is lacking in the chromosome of the recombinant Listeria strain. In another embodiment, the recombinant Listeria vaccine strain further comprises a nucleic acid molecule comprising a third open reading frame encoding a metabolic enzyme, and wherein the metabolic enzyme complements an endogenous gene that is lacking in the chromosome of the recombinant Listeria strain.

In another embodiment, provided herein is a recombinant Listeria vaccine strain comprising a nucleic acid molecule, wherein the nucleic acid molecule comprises a first open reading frame encoding a polypeptide, wherein the polypeptide comprises a Her2/neu chimeric antigen, wherein the nucleic acid molecule further comprises a second and a third open reading frame each encoding a metabolic enzyme, and wherein the metabolic enzyme complements an endogenous gene that is lacking in the chromosome of said recombinant Listeria strain. In one embodiment, the nucleic acid molecule is integrated into the Listeria genome. In another embodiment, the nucleic acid molecule is in a plasmid in the recombinant Listeria vaccine strain. In yet another embodiment, the plasmid is stably maintained in the recombinant Listeria vaccine strain in the absence of antibiotic selection. In another embodiment, the plasmid does not confer antibiotic resistance upon the recombinant Listeria.

In another embodiment, the recombinant Listeria strain is attenuated. In another embodiment, the recombinant Listeria is an attenuated auxotrophic strain. In another embodiment, the high metabolic burden that the expression of a foreign antigen exerts on a bacterium such as one of the present invention is also an important mechanism of attenuation.

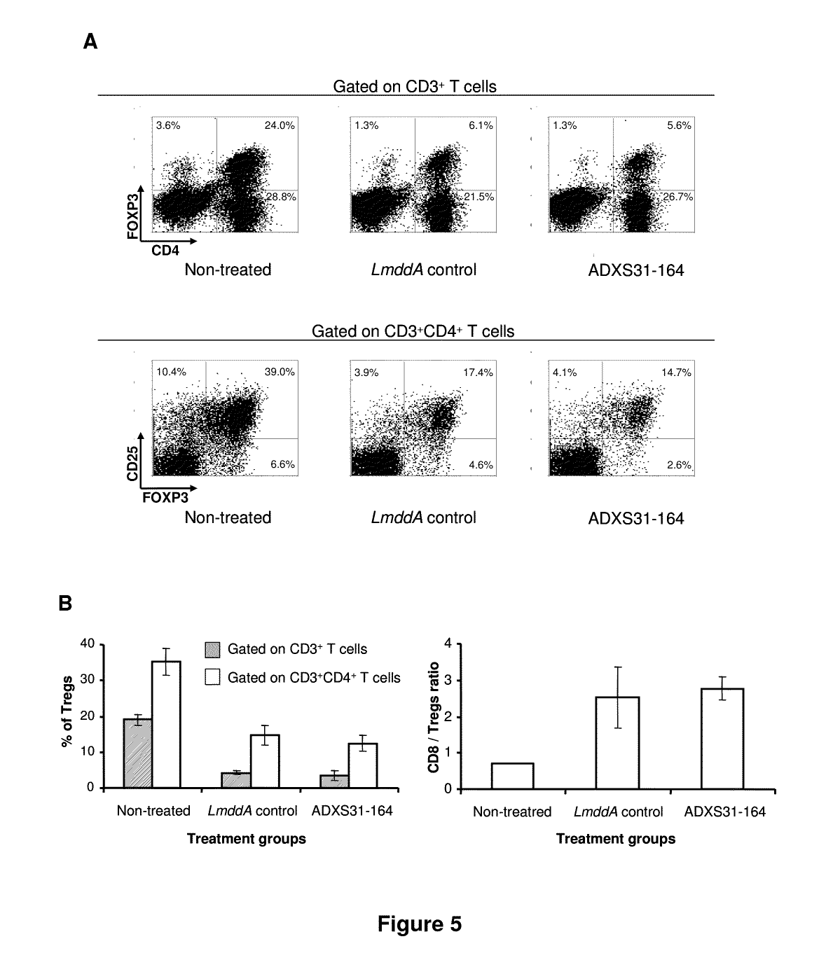

In one embodiment the attenuated strain is LmddA. In another embodiment, this strain exerts a strong adjuvant effect which is an inherent property of Listeria-based vaccines. One manifestation of this adjuvant effect is the 5-fold decrease in the number of the intratumoral Tregs caused by either Listeria expressing an antigen other than chimeric Her-2/neu or the ADXS-31-164 (expressing chimeric Her-2/neu) vaccines (see FIG. 5 herein). In another embodiment, the LmddA vector expressing a different antigen (HPV 16 E7) is also associated with a significant decrease in the frequency of Tregs in the tumors, likely as a consequence of innate immunity responses. In another embodiment, the LmddA vector expresses a prostate-specific antigen (PSA), a human papilloma virus (HPV) antigen (E6, E7). In another embodiment, the HPV strain is HPV 16, HPV 18, or any strain known in the art.

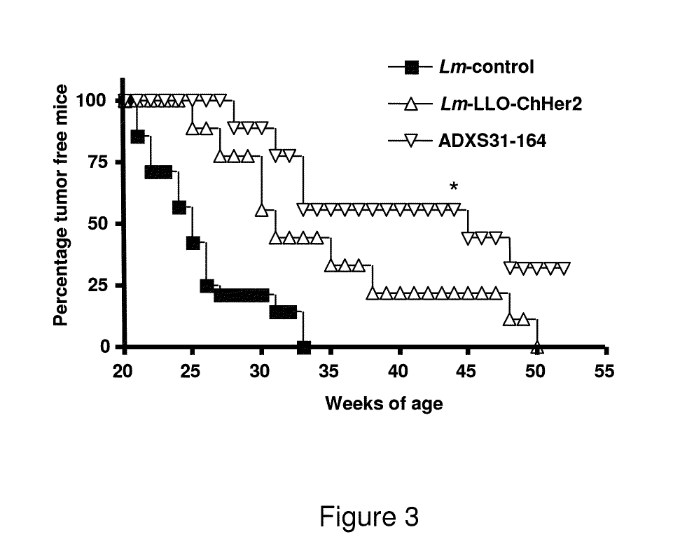

In one embodiment, the attenuated auxotrophic Listeria vaccine strain is the ADXS-31-164 strain. ADXS-31-164 is based on a Listeria vaccine vector which is attenuated due to the deletion of virulence gene actA and retains the plasmid for Her2/neu expression in vivo and in vitro by complementation of dal gene. In one embodiment, ADXS31-164 expresses and secretes the chimeric Her2/neu protein fused to the first 441 amino acids of listeriolysin O (LLO). In another embodiment, ADXS31-164 exerts strong and antigen specific anti-tumor responses with ability to break tolerance toward HER2/neu in transgenic animals (see Examples). In another embodiment, the ADXS31-164 strain is highly attenuated and has a better safety profile than previous Listeria vaccine generation, as it is more rapidly cleared from the spleens of the immunized mice. In another embodiment, the ADXS31-164 results in a longer delay of tumor onset in transgenic animals than Lm-LLO-ChHer2, the antibiotic resistant and more virulent version of this vaccine (see FIG. 3). In another embodiment, ADXS31-164 strain is highly immunogenic, able to break tolerance toward the HER2/neu self-antigen and prevent tumor formation in Her2/neu transgenic animals. In another embodiment, ADXS31-164 causes a significant decrease in intra-tumoral T regulatory cells (Tregs). In another embodiment, the lower frequency of Tregs in tumors treated with LmddA vaccines resulted in an increased intratumoral CD8/Tregs ratio, suggesting that a more favorable tumor microenvironment can be obtained after immunization with LmddA vaccines. In another embodiment, the use of this chimeric antigen does not result in escape mutations indicating that tumors do not mutate away from a therapeutic efficacious response to treatment with this novel antigen (see example 6). In another embodiment, peripheral immunization with ADXS31-164 delays the growth of a metastatic breast cancer cell line in the brain (see Example 7).

In one embodiment, the Lm-LLO-ChHer2 strain is Lm-LLO-138.

In one embodiment, recombinant attenuated, antibiotic-free Listeria-expressing chimeric antigens are useful for preventing, and treating a cancer or solid tumors, as exemplified herein. In another embodiment, the tumor is a Her2/neu positive tumor. In another embodiment, the cancer is a Her2/neu-expressing cancer. In another embodiment, the cancer is breast cancer, a central nervous system (CNS) cancer, a head and neck cancer, an osteosarcoma, a canine osteosarcoma, Ewing's sarcoma (ES), or any Her2/neu-expressing cancer known in the art. In another embodiment, the tumor is an osteo tumor, a breast tumor, a head and neck tumor, or any other antigen-expressing tumor known in the art. In another embodiment, recombinant Listeria expressing a chimeric Her2/neu are useful as a therapeutic vaccine for the treatment of Her2/neu overexpressing solid tumors. In another embodiment, the Her2/neu chimeric antigen provided herein is useful for treating Her2/neu-expressing tumors and preventing escape mutations of the same. In another embodiment, the term “escape mutation” refers to a tumor mutating away from a therapeutic efficacious response to treatment.

In one embodiment, provided herein is a nucleic acid molecule comprising a first open reading frame encoding a recombinant polypeptide provided herein, wherein the nucleic molecule resides within the recombinant Listeria vaccine strain. In another embodiment, the nucleic acid molecule provided herein is used to transform the Listeria in order to arrive at a recombinant Listeria. In another embodiment, the nucleic acid provided herein lacks a virulence gene. In another embodiment, the nucleic acid molecule integrated into the Listeria genome carries a non-functional virulence gene. In another embodiment, the virulence gene is mutated in the genome of the recombinant Listeria. In yet another embodiment, the nucleic acid molecule is used to inactivate the endogenous gene present in the Listeria genome. In yet another embodiment, the virulence gene is an ActA gene. In another embodiment, the virulence gene is a PrfA gene. As will be understood by a skilled artisan, the virulence gene can be any gene known in the art to be associated with virulence in the recombinant Listeria.

In one embodiment, the metabolic gene, the virulence gene, etc. is lacking in a chromosome of the Listeria strain. In another embodiment, the metabolic gene, virulence gene, etc. is lacking in the chromosome and in any episomal genetic element of the Listeria strain. It will be appreciated by a skilled artisan that the term “episome”, “episomal”, etc. refer to a plasmid vector or use thereof that does not integrate into the chromosome of the Listeria provided herein. In another embodiment, the term refers to plasmid vectors that integrate into the chromosome of the Listeria provided herein. In another embodiment, the metabolic gene, virulence gene, etc. is lacking in the genome of the virulence strain. In one embodiment, the virulence gene is mutated in the chromosome. In another embodiment, the virulence gene is deleted from the chromosome.

In one embodiment, the metabolic gene, the virulence gene, etc. is lacking in a chromosome of the Listeria strain. In another embodiment, the metabolic gene, virulence gene, etc. is lacking in the chromosome and in any episomal genetic element of the Listeria strain. In another embodiment, the metabolic gene, virulence gene, etc. is lacking in the genome of the virulence strain. In one embodiment, the virulence gene is mutated in the chromosome. In another embodiment, the virulence gene is deleted from the chromosome.

In another embodiment, the nucleic acids and plasmids provided herein do not confer antibiotic resistance upon the recombinant Listeria.

“Nucleic acid molecule” refers, in another embodiment, to a plasmid. In another embodiment, the term refers to an integration vector. In another embodiment, the term refers to a non-integration vector. In another embodiment, the term refers to a plasmid comprising an integration vector. In another embodiment, the integration vector is a site-specific integration vector. In another embodiment, a nucleic acid molecule of methods and compositions of the present invention are composed of any type of nucleotide known in the art. Each possibility represents a separate embodiment of the present invention.

“Metabolic enzyme” refers, in another embodiment, to an enzyme involved in synthesis of a nutrient required by the host bacteria. In another embodiment, the term refers to an enzyme required for synthesis of a nutrient required by the host bacteria. In another embodiment, the term refers to an enzyme involved in synthesis of a nutrient utilized by the host bacteria. In another embodiment, the term refers to an enzyme involved in synthesis of a nutrient required for sustained growth of the host bacteria. In another embodiment, the enzyme is required for synthesis of the nutrient. Each possibility represents a separate embodiment of the present invention.

“Stably maintained” refers, in another embodiment, to maintenance of a nucleic acid molecule or plasmid in the absence of selection (e.g. antibiotic selection) for 10 generations, without detectable loss. In another embodiment, the period is 15 generations. In another embodiment, the period is 20 generations. In another embodiment, the period is 25 generations. In another embodiment, the period is 30 generations. In another embodiment, the period is 40 generations. In another embodiment, the period is 50 generations. In another embodiment, the period is 60 generations. In another embodiment, the period is 80 generations. In another embodiment, the period is 100 generations. In another embodiment, the period is 150 generations. In another embodiment, the period is 200 generations. In another embodiment, the period is 300 generations. In another embodiment, the period is 500 generations. In another embodiment, the period is more than generations. In another embodiment, the nucleic acid molecule or plasmid is maintained stably in vitro (e.g. in culture). In another embodiment, the nucleic acid molecule or plasmid is maintained stably in vivo. In another embodiment, the nucleic acid molecule or plasmid is maintained stably both in vitro and in vitro. Each possibility represents a separate embodiment of the present invention.

In one embodiment, the present invention provides a recombinant Listeria strain expressing the antigen. The present invention also provides recombinant polypeptides comprising a listeriolysin (LLO) protein fragment fused to a Her-2 chimeric protein or fragment thereof, vaccines and immunogenic compositions comprising same, and methods of inducing an anti-Her-2 immune response and treating and vaccinating against a Her-2-expressing tumor, comprising the same.

In another embodiment, a recombinant Listeria strain of the present invention has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the Listeria strain contains a genomic insertion of the gene encoding the antigen-containing recombinant peptide. In another embodiment, the Listeria strain carries a plasmid comprising the gene encoding the antigen-containing recombinant peptide. In another embodiment, the passaging is performed by any other method known in the art.

In one embodiment, the polypeptide provided herein is a fusion protein comprising an additional polypeptide selected from the group consisting of: a) non-hemolytic LLO protein or N-terminal fragment, b) a PEST sequence, or c) an ActA fragment, and further wherein said additional polypeptide is fused to the Her2/neu chimeric antigen. In another embodiment, the additional polypeptide is functional. In another embodiment, a fragment of the additional polypeptide is immunogenic. In another embodiment, the additional polypeptide is immunogenic.

In another embodiment, the polypeptide provided herein is a fusion protein comprising a non-hemolytic LLO protein or N-terminal fragment fused to the Her2/neu chimeric antigen. In another embodiment, a fusion protein of methods and compositions of the present invention comprises an ActA sequence from a Listeria organism. ActA proteins and fragments thereof augment antigen presentation and immunity in a similar fashion to LLO.

In another embodiment of methods and compositions of the present invention, the fusion protein comprises the Her2/neu antigen and an additional adjuvant polypeptide In one embodiment, the additional polypeptide is a non-hemolytic LLO protein or fragment thereof (Examples herein). In another embodiment, the additional polypeptide is a PEST sequence. In another embodiment, the additional polypeptide is an ActA protein or a fragment thereof. ActA proteins and fragments thereof augment antigen presentation and immunity in a similar fashion to LLO.

The additional polypeptide of methods and compositions of the present invention is, in another embodiment, a listeriolysin (LLO) peptide. In another embodiment, the additional polypeptide is an ActA peptide. In another embodiment, the additional polypeptide is a PEST-like sequence peptide. In another embodiment, the additional polypeptide is any other peptide capable of enhancing the immunogenicity of an antigen peptide. Each possibility represents a separate embodiment of the present invention.

Fusion proteins comprising the Her2/neu chimeric antigen may be prepared by any suitable method, including, for example, cloning and restriction of appropriate sequences or direct chemical synthesis by methods discussed below. Alternatively, subsequences may be cloned and the appropriate subsequences cleaved using appropriate restriction enzymes. The fragments may then be ligated to produce the desired DNA sequence. In one embodiment, DNA encoding the antigen can be produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The antigen is ligated into a plasmid. Each method represents a separate embodiment of the present invention.

The results of the present invention demonstrate that administration of compositions of the present invention has utility for inducing formation of antigen-specific T cells (e.g. cytotoxic T cells) that recognize and kill tumor cells (Examples herein).

In one embodiment, the present invention provides a recombinant polypeptide comprising an N-terminal fragment of an LLO protein fused to a Her-2 chimeric protein or fused to a fragment thereof. In one embodiment, the present invention provides a recombinant polypeptide consisting of an N-terminal fragment of an LLO protein fused to a Her-2 chimeric protein or fused to a fragment thereof.

In another embodiment, the Her-2 chimeric protein of the methods and compositions of the present invention is a human Her-2 chimeric protein. In another embodiment, the Her-2 protein is a mouse Her-2 chimeric protein. In another embodiment, the Her-2 protein is a rat Her-2 chimeric protein. In another embodiment, the Her-2 protein is a primate Her-2 chimeric protein. In another embodiment, the Her-2 protein is a Her-2 chimeric protein of human or any other animal species or combinations thereof known in the art. Each possibility represents a separate embodiment of the present invention.

In another embodiment, a Her-2 protein is a protein referred to as “HER-2/neu,” “Erbb2,” “v-erb-b2,” “c-erb-b2,” “neu,” or “cNeu.” Each possibility represents a separate embodiment of the present invention.

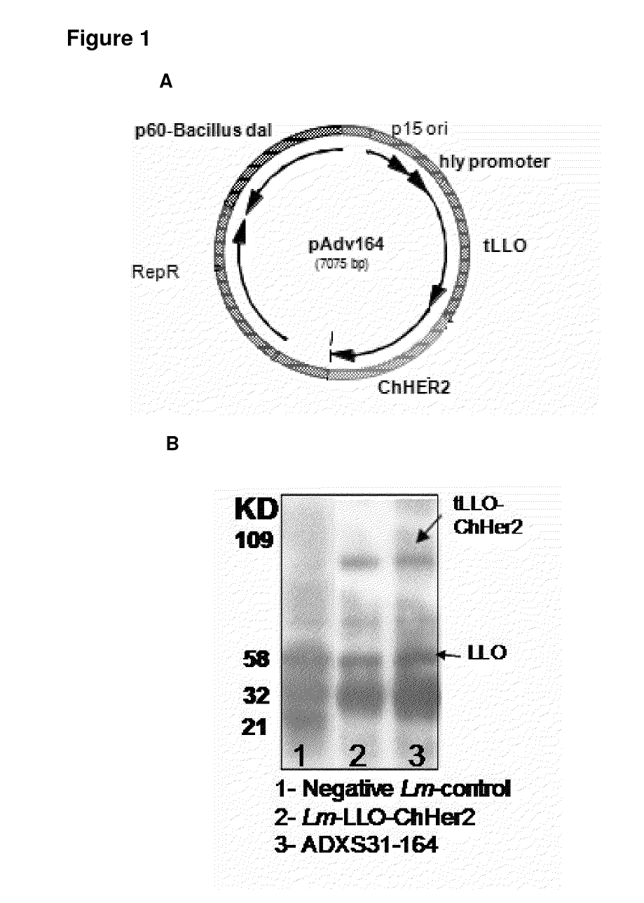

In one embodiment, the Her2-neu chimeric protein, harbors two of the extracellular and one intracellular fragments of Her2/neu antigen showing clusters of MHC-class I epitopes of the oncogene, where, in another embodiment, the chimeric protein, harbors 3 H2Dq and at least 17 of the mapped human MHC-class I epitopes of the Her2/neu antigen (fragments EC1, EC2, and IC1) (See FIG. 1). In another embodiment, the chimeric protein harbors at least 13 of the mapped human MHC-class I epitopes (fragments EC2 and IC1). In another embodiment, the chimeric protein harbors at least 14 of the mapped human MHC-class I epitopes (fragments EC1 and IC1). In another embodiment, the chimeric protein harbors at least 9 of the mapped human MHC-class I epitopes (fragments EC1 and IC2). In another embodiment, the Her2-neu chimeric protein is fused to a non-hemolytic listeriolysin O (LLO). In another embodiment, the Her2-neu chimeric protein is fused to the first 441 amino acids of the Listeria-monocytogenes listeriolysin O (LLO) protein and expressed and secreted by the Listeria monocytogenes attenuated auxotrophic strain LmddA. In another embodiment, the expression and secretion of the fusion protein tLLO-ChHer2 from the attenuated auxotrophic strain provided herein that expresses a chimeric Her2/neu antigen/LLO fusion protein is comparable to that of the Lm-LLO-ChHer2 in TCA precipitated cell culture supernatants after 8 hours of in vitro growth (See FIG. 1B).

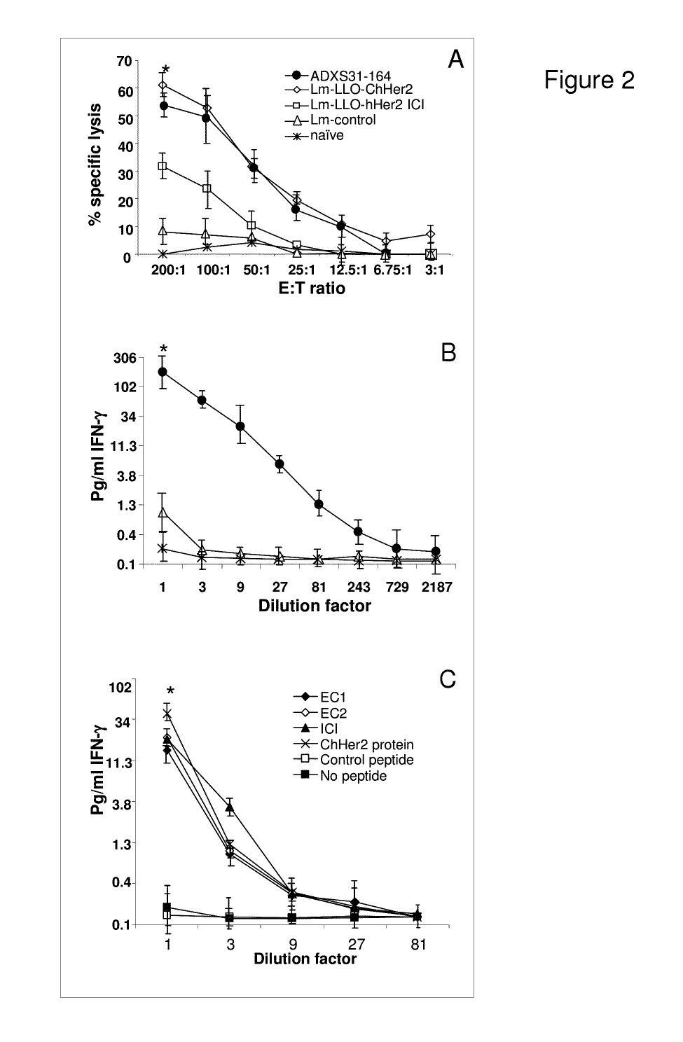

In one embodiment, no CTL activity is detected in naïve animals or mice injected with an irrelevant Listeria vaccine (See FIG. 2A). While in another embodiment, the attenuated auxotrophic strain (ADXS31-164) provided herein is able to stimulate the secretion of IFN-γ by the splenocytes from wild type FVB/N mice (FIG. 2B).

In another embodiment, the metabolic enzyme of the methods and compositions provided herein is an amino acid metabolism enzyme, where, in another embodiment, the metabolic enzyme is an alanine racemase enzyme. In another embodiment, the metabolic enzyme is a D-amino acid transferase enzyme. In another embodiment, the metabolic enzyme catalyzes a formation of an amino acid used for a cell wall synthesis in the recombinant Listeria strain, where in another embodiment, the metabolic enzyme is an alanine racemase enzyme.

In another embodiment, the gene encoding the metabolic enzyme is expressed under the control of the Listeria p60 promoter. In another embodiment, the inlA (encodes internalin) promoter is used. In another embodiment, the hly promoter is used. In another embodiment, the ActA promoter is used. In another embodiment, the integrase gene is expressed under the control of any other gram positive promoter. In another embodiment, the gene encoding the metabolic enzyme is expressed under the control of any other promoter that functions in Listeria. The skilled artisan will appreciate that other promoters or polycistronic expression cassettes may be used to drive the expression of the gene. Each possibility represents a separate embodiment of the present invention.

In another embodiment, the Her-2 chimeric protein is encoded by the following nucleic acid sequence set forth in SEQ ID NO:1

(SEQ ID NO: 1)

gagacccacctggacatgctccgccacctctaccagggctgccaggtggt
gcagggaaacctggaactcacctacctgcccaccaatgccagcctgtcct
tcctgcaggatatccaggaggtgcagggctacgtgctcatcgctcacaac
caagtgaggcaggtcccactgcagaggctgcggattgtgcgaggcaccca
gctctttgaggacaactatgccctggccgtgctagacaatggagacccgc
tgaacaataccacccctgtcacaggggcctccccaggaggcctgcgggag
ctgcagcttcgaagcctcacagagatcttgaaaggaggggtcttgatcca
gcggaacccccagctctgctaccaggacacgattttgtggaagaatatcc
aggagtttgctggctgcaagaagatctttgggagcctggcatttctgccg
gagagctttgatggggacccagcctccaacactgccccgctccagccaga
gcagctccaagtgtttgagactctggaagagatcacaggttacctataca
tctcagcatggccggacagcctgcctgacctcagcgtcttccagaacctg
caagtaatccggggacgaattctgcacaatggcgcctactcgctgaccct
gcaagggctgggcatcagctggctggggctgcgctcactgagggaactgg
gcagtggactggccctcatccaccataacacccacctctgcttcgtgcac
acggtgccctgggaccagctctttcggaacccgcaccaagctctgctcca
cactgccaaccggccagaggacgagtgtgtgggcgagggcctggcctgcc
accagctgtgcgcccgagggcagcagaagatccggaagtacacgatgcgg
agactgctgcaggaaacggagctggtggagccgctgacacctagcggagc
gatgcccaaccaggcgcagatgcggatcctgaaagagacggagctgagga
aggtgaaggtgcttggatctggcgcttttggcacagtctacaagggcatc
tggatccctgatggggagaatgtgaaaattccagtggccatcaaagtgtt
gagggaaaacacatcccccaaagccaacaaagaaatcttagacgaagcat
acgtgatggctggtgtgggctccccatatgtctcccgccttctgggcatc
tgcctgacatccacggtgcagctggtgacacagcttatgccctatggctg
cctcttagactaa.

In another embodiment, the Her-2 chimeric protein has the sequence:

(SEQ ID NO: 2)
E T H L D M L R H L Y Q G C Q V V Q G N L E L T Y L P T N A S L S
F L Q D I Q E V Q G Y V L I A H N Q V R Q V P L Q R L R I V R G T
Q L F E D N Y A L A V L D N G D P L N N T T P V T G A S P G G L R
E L Q L R S L T E I L K G G V L I Q R N P Q L C Y Q D T I L W K N I
Q E F A G C K K I F G S L A F L P E S F D G D P A S N T A P L Q P E
Q L Q V F E T L E E I T G Y L Y I S A W P D S L P D L S V F Q N L Q
V I R G R I L H N G A Y S L T L Q G L G I S W L G L R S L R E L G S
G L A L I H H N T H L C F V H T V P W D Q L F R N P H Q A L L H T
A N R P E D E C V G E G L A C H Q L C A R G Q Q K I R K Y T M R
R L L Q E T E L V E P L T P S G A M P N Q A Q M R I L K E T E L R
K V K V L G S G A F G T V Y K G I W I P D G E N V K I P V A I K V
L R E N T S P K A N K E I L D E A Y V M A G V G S P Y V S R L L G I C
L T S T V Q L V T Q L M P Y G C L L D.

In one embodiment, the Her2 chimeric protein or fragment thereof of the methods and compositions provided herein does not include a signal sequence thereof. In another embodiment, omission of the signal sequence enables the Her2 fragment to be successfully expressed in Listeria, due the high hydrophobicity of the signal sequence. Each possibility represents a separate embodiment of the present invention.

In another embodiment, the fragment of a Her2 chimeric protein of methods and compositions of the present invention does not include a transmembrane domain (TM) thereof. In one embodiment, omission of the TM enables the Her-2 fragment to be successfully expressed in Listeria, due the high hydrophobicity of the TM. Each possibility represents a separate embodiment of the present invention.

In one embodiment, the nucleic acid sequence of rat-Her2/neu gene is

(SEQ ID NO: 45)

CCGGAATCGCGGGCACCCAAGTGTGTACCGGCACAGACATGAAGTTGCGG
CTCCCTGCCAGTCCTGAGACCCACCTGGACATGCTCCGCCACCTGTACCA
GGGCTGTCAGGTAGTGCAGGGCAACTTGGAGCTTACCTACGTGCCTGCCA
ATGCCAGCCTCTCATTCCTGCAGGACATCCAGGAAGTTCAGGGTTACATG
CTCATCGCTCACAACCAGGTGAAGCGCGTCCCACTGCAAAGGCTGCGCAT
CGTGAGAGGGACCCAGCTCTTTGAGGACAAGTATGCCCTGGCTGTGCTAG
ACAACCGAGATCCTCAGGACAATGTCGCCGCCTCCACCCCAGGCAGAACC
CCAGAGGGGCTGCGGGAGCTGCAGCTTCGAAGTCTCACAGAGATCCTGAA
GGGAGGAGTTTTGATCCGTGGGAACCCTCAGCTCTGCTACCAGGACATGG
TTTTGTGGAAGGACGTCTTCCGCAAGAATAACCAACTGGCTCCTGTCGAT
ATAGACACCAATCGTTCCCGGGCCTGTCCACCTTGTGCCCCCGCCTGCAA
AGACAATCACTGTTGGGGTGAGAGTCCGGAAGACTGTCAGATCTTGACTG
GCACCATCTGTACCAGTGGTTGTGCCCGGTGCAAGGGCCGGCTGCCCACT
GACTGCTGCCATGAGCAGTGTGCCGCAGGCTGCACGGGCCCCAAGCATTC
TGACTGCCTGGCCTGCCTCCACTTCAATCATAGTGGTATCTGTGAGCTGC
ACTGCCCAGCCCTCGTCACCTACAACACAGACACCTTTGAGTCCATGCAC
AACCCTGAGGGTCGCTACACCTTTGGTGCCAGCTGCGTGACCACCTGCCC
CTACAACTACCTGTCTACGGAAGTGGGATCCTGCACTCTGGTGTGTCCCC
CGAATAACCAAGAGGTCACAGCTGAGGACGGAACACAGCGTTGTGAGAAA
TGCAGCAAGCCCTGTGCTCGAGTGTGCTATGGTCTGGGCATGGAGCACCT
TCGAGGGGCGAGGGCCATCACCAGTGACAATGTCCAGGAGTTTGATGGCT
GCAAGAAGATCTTTGGGAGCCTGGCATTTTTGCCGGAGAGCTTTGATGGG
GACCCCTCCTCCGGCATTGCTCCGCTGAGGCCTGAGCAGCTCCAAGTGTT
CGAAACCCTGGAGGAGATCACAGGTTACCTGTACATCTCAGCATGGCCAG
ACAGTCTCCGTGACCTCAGTGTCTTCCAGAACCTTCGAATCATTCGGGGA
CGGATTCTCCACGATGGCGCGTACTCATTGACACTGCAAGGCCTGGGGAT
CCACTCGCTGGGGCTGCGCTCACTGCGGGAGCTGGGCAGTGGATTGGCTC
TGATTCACCGCAACGCCCATCTCTGCTTTGTACACACTGTACCTTGGGAC
CAGCTCTTCCGGAACCCACATCAGGCCCTGCTCCACAGTGGGAACCGGCC
GGAAGAGGATTGTGGTCTCGAGGGCTTGGTCTGTAACTCACTGTGTGCCC
ACGGGCACTGCTGGGGGCCAGGGCCCACCCAGTGTGTCAACTGCAGTCAT
TTCCTTCGGGGCCAGGAGTGTGTGGAGGAGTGCCGAGTATGGAAGGGGCT
CCCCCGGGAGTATGTGAGTGACAAGCGCTGTCTGCCGTGTCACCCCGAGT
GTCAGCCTCAAAACAGCTCAGAGACCTGCTTTGGATCGGAGGCTGATCAG
TGTGCAGCCTGCGCCCACTACAAGGACTCGTCCTCCTGTGTGGCTCGCTG
CCCCAGTGGTGTGAAACCGGACCTCTCCTACATGCCCATCTGGAAGTACC
CGGATGAGGAGGGCATATGCCAGCCGTGCCCCATCAACTGCACCCACTCC
TGTGTGGATCTGGATGAACGAGGCTGCCCAGCAGAGCAGAGAGCCAGCCC
GGTGACATTCATCATTGCAACTGTAGTGGGCGTCCTGCTGTTCCTGATCT
TAGTGGTGGTCGTTGGAATCCTAATCAAACGAAGGAGACAGAAGATCCGG
AAGTATACGATGCGTAGGCTGCTGCAGGAAACTGAGTTAGTGGAGCCGCT
GACGCCCAGCGGAGCAATGCCCAACCAGGCTCAGATGCGGATCCTAAAAG
AGACGGAGCTAAGGAAGGTGAAGGTGCTTGGATCAGGAGCTTTTGGCACT
GTCTACAAGGGCATCTGGATCCCAGATGGGGAGAATGTGAAAATCCCCGT
GGCTATCAAGGTGTTGAGAGAAAACACATCTCCTAAAGCCAACAAAGAAA
TTCTAGATGAAGCGTATGTGATGGCTGGTGTGGGTTCTCCGTATGTGTCC
CGCCTCCTGGGCATCTGCCTGACATCCACAGTACAGCTGGTGACACAGCT
TATGCCCTACGGCTGCCTTCTGGACCATGTCCGAGAACACCGAGGTCGCC
TAGGCTCCCAGGACCTGCTCAACTGGTGTGTTCAGATTGCCAAGGGGATG
AGCTACCTGGAGGACGTGCGGCTTGTACACAGGGACCTGGCTGCCCGGAA
TGTGCTAGTCAAGAGTCCCAACCACGTCAAGATTACAGATTTCGGGCTGG
CTCGGCTGCTGGACATTGATGAGACAGAGTACCATGCAGATGGGGGCAAG
GTGCCCATCAAATGGATGGCATTGGAATCTATTCTCAGACGCCGGTTCAC
CCATCAGAGTGATGTGTGGAGCTATGGAGTGACTGTGTGGGAGCTGATGA
CTTTTGGGGCCAAACCTTACGATGGAATCCCAGCCCGGGAGATCCCTGAT
TTGCTGGAGAAGGGAGAACGCCTACCTCAGCCTCCAATCTGCACCATTGA
TGTCTACATGATTATGGTCAAATGTTGGATGATTGACTCTGAATGTCGCC
CGAGATTCCGGGAGTTGGTGTCAGAATTTTCACGTATGGCGAGGGACCCC
CAGCGTTTTGTGGTCATCCAGAACGAGGACTTGGGCCCATCCAGCCCCAT
GGACAGTACCTTCTACCGTTCACTGCTGGAAGATGATGACATGGGTGACC
TGGTAGACGCTGAAGAGTATCTGGTGCCCCAGCAGGGATTCTTCTCCCCG
GACCCTACCCCAGGCACTGGGAGCACAGCCCATAGAAGGCACCGCAGCTC
GTCCACCAGGAGTGGAGGTGGTGAGCTGACACTGGGCCTGGAGCCCTCGG
AAGAAGGGCCCCCCAGATCTCCACTGGCTCCCTCGGAAGGGGCTGGCTCC
GATGTGTTTGATGGTGACCTGGCAATGGGGGTAACCAAAGGGCTGCAGAG
CCTCTCTCCACATGACCTCAGCCCTCTACAGCGGTACAGCGAGGACCCCA
CATTACCTCTGCCCCCCGAGACTGATGGCTATGTTGCTCCCCTGGCCTGC
AGCCCCCAGCCCGAGTATGTGAACCAATCAGAGGTTCAGCCTCAGCCTCC
TTTAACCCCAGAGGGTCCTCTGCCTCCTGTCCGGCCTGCTGGTGCTACTC
TAGAAAGACCCAAGACTCTCTCTCCTGGGAAGAATGGGGTTGTCAAAGAC
GTTTTTGCCTTCGGGGGTGCTGTGGAGAACCCTGAATACTTAGTACCGAG
AGAAGGCACTGCCTCTCCGCCCCACCCTTCTCCTGCCTTCAGCCCAGCCT
TTGACAACCTCTATTACTGGGACCAGAACTCATCGGAGCAGGGGCCTCCA
CCAAGTAACTTTGAAGGGACCCCCACTGCAGAGAACCCTGAGTACCTAGG
CCTGGATGTACCTGTA.

In one embodiment, the nucleic acid sequence encoding the rat/her2/neu EC 1 fragment is

(SEQ ID NO: 46)

CCCAGGCAGAACCCCAGAGGGGCTGCGGGAGCTGCAGCTTCGAAGTCTCA
CAGAGATCCTGAAGGGAGGAGTTTTGATCCGTGGGAACCCTCAGCTCTGC
TACCAGGACATGGTTTTGTGGAAGGACGTCTTCCGCAAGAATAACCAACT
GGCTCCTGTCGATATAGACACCAATCGTTCCCGGGCCTGTCCACCTTGTG
CCCCCGCCTGCAAAGACAATCACTGTTGGGGTGAGAGTCCGGAAGACTGT
CAGATCTTGACTGGCACCATCTGTACCAGTGGTTGTGCCCGGTGCAAGGG
CCGGCTGCCCACTGACTGCTGCCATGAGCAGTGTGCCGCAGGCTGCACGG
GCCCCAAGCA.

In another embodiment, the nucleic acid sequence encoding the rat her2/neu EC2 fragment is:

(SEQ ID NO: 47)

GGTCACAGCTGAGGACGGAACACAGCGTTGTGAGAAATGCAGCAAGCCCT
GTGCTCGAGTGTGCTATGGTCTGGGCATGGAGCACCTTCGAGGGGCGAGG
GCCATCACCAGTGACAATGTCCAGGAGTTTGATGGCTGCAAGAAGATCTT
TGGGAGCCTGGCATTTTTGCCGGAGAGCTTTGATGGGGACCCCTCCTCCG
GCATTGCTCCGCTGAGGCCTGAGCAGCTCCAAGTGTTCGAAACCCTGGAG
GAGATCACAGGTTACCTGTACATCTCAGCATGGCCAGACAGTCTCCGTGA
CCTCAGTGTCTTCCAGAACCTTCGAATCATTCGGGGACGGATTCTCCACG
ATGGCGCGTACTCATTGACACTGCAAGGCCTGGGGATCCACTCGCTGGGG
CTGCGCTCACTGCGGGAGCTGGGCAGTGGATTGGCTCTGATTCACCGCAA
CGCCCATCTCTGCTTTGTACACACTGTACCTTGGGACCAGCTCTTCCGGA
ACCCACATCAGGCCCTGCTCCACAGTGGGAACCGGCCGGAAGAGGATTGT
GGTCTCGAGGGCTTGGTCTGTAACTCACTGTGTGCCCACGGGCACTGCTG
GGGGCCAGGGCCCACCCA.

In another embodiment, the nucleic acid sequence encoding the rat her2/neu IC 1 fragment is:

(SEQ ID NO: 48)

CGCCCAGCGGAGCAATGCCCAACCAGGCTCAGATGCGGATCCTAAAAGAG
ACGGAGCTAAGGAAGGTGAAGGTGCTTGGATCAGGAGCTTTTGGCACTGT
CTACAAGGGCATCTGGATCCCAGATGGGGAGAATGTGAAAATCCCCGTGG
CTATCAAGGTGTTGAGAGAAAACACATCTCCTAAAGCCAACAAAGAAATT
CTAGATGAAGCGTATGTGATGGCTGGTGTGGGTTCTCCGTATGTGTCCCG
CCTCCTGGGCATCTGCCTGACATCCACAGTACAGCTGGTGACACAGCTTA
TGCCCTACGGCTGCCTTCTGGACCATGTCCGAGAACACCGAGGTCGCCTA
GGCTCCCAGGACCTGCTCAACTGGTGTGTTCAGATTGCCAAGGGGATGAG
CTACCTGGAGGACGTGCGGCTTGTACACAGGGACCTGGCTGCCCGGAATG
TGCTAGTCAAGAGTCCCAACCACGTCAAGATTACAGATTTCGGGCTGGCT
CGGCTGCTGGACATTGATGAGACAGAGTACCATGCAGATGGGGGCAAGGT
GCCCATCAAATGGATGGCATTGGAATCTATTCTCAGACGCCGGTTCACCC
ATCAGAGTGATGTGTGGAGCTATGGAGTGACTGTGTGGGAGCTGATGACT
TTTGGGGCCAAACCTTACGATGGAATCCCAGCCCGGGAGATCCCTGATTT
GCTGGAGAAGGGAGAACGCCTACCTCAGCCTCCAATCTGCACCATTGATG
TCTACATGATTATGGTCAAATGTTGGATGATTGACTCTGAATGTCGCCCG
AGATTCCGGGAGTTGGTGTCAGAATTTTCACGTATGGCGAGGGACCCCCA
GCGTTTTGTGGTCATCCAGAACGAGGACTTGGGCCCATCCAGCCCCATGG
ACAGTACCTTCTACCGTTCACTGCTGGAA.

In one embodiment, the nucleic acid sequence of human-Her2/neu gene is:

(SEQ ID NO: 49)

ATGGAGCTGGCGGCCTTGTGCCGCTGGGGGCTCCTCCTCGCCCTCTTGCC
CCCCGGAGCCGCGAGCACCCAAGTGTGCACCGGCACAGACATGAAGCTGC
GGCTCCCTGCCAGTCCCGAGACCCACCTGGACATGCTCCGCCACCTCTAC
CAGGGCTGCCAGGTGGTGCAGGGAAACCTGGAACTCACCTACCTGCCCAC
CAATGCCAGCCTGTCCTTCCTGCAGGATATCCAGGAGGTGCAGGGCTACG
TGCTCATCGCTCACAACCAAGTGAGGCAGGTCCCACTGCAGAGGCTGCGG
ATTGTGCGAGGCACCCAGCTCTTTGAGGACAACTATGCCCTGGCCGTGCT
AGACAATGGAGACCCGCTGAACAATACCACCCCTGTCACAGGGGCCTCCC
CAGGAGGCCTGCGGGAGCTGCAGCTTCGAAGCCTCACAGAGATCTTGAAA
GGAGGGGTCTTGATCCAGCGGAACCCCCAGCTCTGCTACCAGGACACGAT
TTTGTGGAAGGACATCTTCCACAAGAACAACCAGCTGGCTCTCACACTGA
TAGACACCAACCGCTCTCGGGCCTGCCACCCCTGTTCTCCGATGTGTAAG
GGCTCCCGCTGCTGGGGAGAGAGTTCTGAGGATTGTCAGAGCCTGACGCG
CACTGTCTGTGCCGGTGGCTGTGCCCGCTGCAAGGGGCCACTGCCCACTG
ACTGCTGCCATGAGCAGTGTGCTGCCGGCTGCACGGGCCCCAAGCACTCT
GACTGCCTGGCCTGCCTCCACTTCAACCACAGTGGCATCTGTGAGCTGCA
CTGCCCAGCCCTGGTCACCTACAACACAGACACGTTTGAGTCCATGCCCA
ATCCCGAGGGCCGGTATACATTCGGCGCCAGCTGTGTGACTGCCTGTCCC
TACAACTACCTTTCTACGGACGTGGGATCCTGCACCCTCGTCTGCCCCCT
GCACAACCAAGAGGTGACAGCAGAGGATGGAACACAGCGGTGTGAGAAGT
GCAGCAAGCCCTGTGCCCGAGTGTGCTATGGTCTGGGCATGGAGCACTTG
CGAGAGGTGAGGGCAGTTACCAGTGCCAATATCCAGGAGTTTGCTGGCTG
CAAGAAGATCTTTGGGAGCCTGGCATTTCTGCCGGAGAGCTTTGATGGGG
ACCCAGCCTCCAACACTGCCCCGCTCCAGCCAGAGCAGCTCCAAGTGTTT
GAGACTCTGGAAGAGATCACAGGTTACCTATACATCTCAGCATGGCCGGA
CAGCCTGCCTGACCTCAGCGTCTTCCAGAACCTGCAAGTAATCCGGGGAC
GAATTCTGCACAATGGCGCCTACTCGCTGACCCTGCAAGGGCTGGGCATC
AGCTGGCTGGGGCTGCGCTCACTGAGGGAACTGGGCAGTGGACTGGCCCT
CATCCACCATAACACCCACCTCTGCTTCGTGCACACGGTGCCCTGGGACC
AGCTCTTTCGGAACCCGCACCAAGCTCTGCTCCACACTGCCAACCGGCCA
GAGGACGAGTGTGTGGGCGAGGGCCTGGCCTGCCACCAGCTGTGCGCCCG
AGGGCACTGCTGGGGTCCAGGGCCCACCCAGTGTGTCAACTGCAGCCAGT
TCCTTCGGGGCCAGGAGTGCGTGGAGGAATGCCGAGTACTGCAGGGGCTC
CCCAGGGAGTATGTGAATGCCAGGCACTGTTTGCCGTGCCACCCTGAGTG
TCAGCCCCAGAATGGCTCAGTGACCTGTTTTGGACCGGAGGCTGACCAGT
GTGTGGCCTGTGCCCACTATAAGGACCCTCCCTTCTGCGTGGCCCGCTGC
CCCAGCGGTGTGAAACCTGACCTCTCCTACATGCCCATCTGGAAGTTTCC
AGATGAGGAGGGCGCATGCCAGCCTTGCCCCATCAACTGCACCCACTCCT
GTGTGGACCTGGATGACAAGGGCTGCCCCGCCGAGCAGAGAGCCAGCCCT
CTGACGTCCATCGTCTCTGCGGTGGTTGGCATTCTGCTGGTCGTGGTCTT
GGGGGTGGTCTTTGGGATCCTCATCAAGCGACGGCAGCAGAAGATCCGGA
AGTACACGATGCGGAGACTGCTGCAGGAAACGGAGCTGGTGGAGCCGCTG
ACACCTAGCGGAGCGATGCCCAACCAGGCGCAGATGCGGATCCTGAAAGA
GACGGAGCTGAGGAAGGTGAAGGTGCTTGGATCTGGCGCTTTTGGCACAG
TCTACAAGGGCATCTGGATCCCTGATGGGGAGAATGTGAAAATTCCAGTG
GCCATCAAAGTGTTGAGGGAAAACACATCCCCCAAAGCCAACAAAGAAAT
CTTAGACGAAGCATACGTGATGGCTGGTGTGGGCTCCCCATATGTCTCCC
GCCTTCTGGGCATCTGCCTGACATCCACGGTGCAGCTGGTGACACAGCTT
ATGCCCTATGGCTGCCTCTTAGACCATGTCCGGGAAAACCGCGGACGCCT
GGGCTCCCAGGACCTGCTGAACTGGTGTATGCAGATTGCCAAGGGGATGA
GCTACCTGGAGGATGTGCGGCTCGTACACAGGGACTTGGCCGCTCGGAAC
GTGCTGGTCAAGAGTCCCAACCATGTCAAAATTACAGACTTCGGGCTGGC
TCGGCTGCTGGACATTGACGAGACAGAGTACCATGCAGATGGGGGCAAGG
TGCCCATCAAGTGGATGGCGCTGGAGTCCATTCTCCGCCGGCGGTTCACC
CACCAGAGTGATGTGTGGAGTTATGGTGTGACTGTGTGGGAGCTGATGAC
TTTTGGGGCCAAACCTTACGATGGGATCCCAGCCCGGGAGATCCCTGACC
TGCTGGAAAAGGGGGAGCGGCTGCCCCAGCCCCCCATCTGCACCATTGAT
GTCTACATGATCATGGTCAAATGTTGGATGATTGACTCTGAATGTCGGCC
AAGATTCCGGGAGTTGGTGTCTGAATTCTCCCGCATGGCCAGGGACCCCC
AGCGCTTTGTGGTCATCCAGAATGAGGACTTGGGCCCAGCCAGTCCCTTG
GACAGCACCTTCTACCGCTCACTGCTGGAGGACGATGACATGGGGGACCT
GGTGGATGCTGAGGAGTATCTGGTACCCCAGCAGGGCTTCTTCTGTCCAG
ACCCTGCCCCGGGCGCTGGGGGCATGGTCCACCACAGGCACCGCAGCTCA
TCTACCAGGAGTGGCGGTGGGGACCTGACACTAGGGCTGGAGCCCTCTGA
AGAGGAGGCCCCCAGGTCTCCACTGGCACCCTCCGAAGGGGCTGGCTCCG
ATGTATTTGATGGTGACCTGGGAATGGGGGCAGCCAAGGGGCTGCAAAGC
CTCCCCACACATGACCCCAGCCCTCTACAGCGGTACAGTGAGGACCCCAC
AGTACCCCTGCCCTCTGAGACTGATGGCTACGTTGCCCCCCTGACCTGCA
GCCCCCAGCCTGAATATGTGAACCAGCCAGATGTTCGGCCCCAGCCCCCT
TCGCCCCGAGAGGGCCCTCTGCCTGCTGCCCGACCTGCTGGTGCCACTCT
GGAAAGGGCCAAGACTCTCTCCCCAGGGAAGAATGGGGTCGTCAAAGACG
TTTTTGCCTTTGGGGGTGCCGTGGAGAACCCCGAGTACTTGACACCCCAG
GGAGGAGCTGCCCCTCAGCCCCACCCTCCTCCTGCCTTCAGCCCAGCCTT
CGACAACCTCTATTACTGGGACCAGGACCCACCAGAGCGGGGGGCTCCAC
CCAGCACCTTCAAAGGGACACCTACGGCAGAGAACCCAGAGTACCTGGGT
CTGGACGTGCCAGTGTGAACCAGAAGGCCAAGTCCGCAGAAGCCCTGA.

In another embodiment, the nucleic acid sequence encoding the human her2/neu EC 1 fragment implemented into the chimera spans from 120-510 bp of the human EC 1 region and is set forth in (SEQ ID NO: 50).

(SEQ ID NO: 50)

GAGACCCACCTGGACATGCTCCGCCACCTCTACCAGGGCTGCCAGGTGGT
GCAGGGAAACCTGGAACTCACCTACCTGCCCACCAATGCCAGCCTGTCCT
TCCTGCAGGATATCCAGGAGGTGCAGGGCTACGTGCTCATCGCTCACAAC
CAAGTGAGGCAGGTCCCACTGCAGAGGCTGCGGATTGTGCGAGGCACCCA
GCTCTTTGAGGACAACTATGCCCTGGCCGTGCTAGACAATGGAGACCCGC
TGAACAATACCACCCCTGTCACAGGGGCCTCCCCAGGAGGCCTGCGGGAG
CTGCAGCTTCGAAGCCTCACAGAGATCTTGAAAGGAGGGGTCTTGATCCA
GCGGAACCCCCAGCTCTGCTACCAGGACACGATTTTGTGGAAG.

In one embodiment, the complete EC1 human her2/neu fragment spans from (58-979 bp of the human her2/neu gene and is set forth in (SEQ ID NO: 54).

(SEQ ID NO: 54)

GCCGCGAGCACCCAAGTGTGCACCGGCACAGACATGAAGCTGCGGCTCCC
TGCCAGTCCCGAGACCCACCTGGACATGCTCCGCCACCTCTACCAGGGCT
GCCAGGTGGTGCAGGGAAACCTGGAACTCACCTACCTGCCCACCAATGCC
AGCCTGTCCTTCCTGCAGGATATCCAGGAGGTGCAGGGCTACGTGCTCAT
CGCTCACAACCAAGTGAGGCAGGTCCCACTGCAGAGGCTGCGGATTGTGC
GAGGCACCCAGCTCTTTGAGGACAACTATGCCCTGGCCGTGCTAGACAAT
GGAGACCCGCTGAACAATACCACCCCTGTCACAGGGGCCTCCCCAGGAGG
CCTGCGGGAGCTGCAGCTTCGAAGCCTCACAGAGATCTTGAAAGGAGGGG
TCTTGATCCAGCGGAACCCCCAGCTCTGCTACCAGGACACGATTTTGTGG
AAGGACATCTTCCACAAGAACAACCAGCTGGCTCTCACACTGATAGACAC
CAACCGCTCTCGGGCCTGCCACCCCTGTTCTCCGATGTGTAAGGGCTCCC
GCTGCTGGGGAGAGAGTTCTGAGGATTGTCAGAGCCTGACGCGCACTGTC
TGTGCCGGTGGCTGTGCCCGCTGCAAGGGGCCACTGCCCACTGACTGCTG
CCATGAGCAGTGTGCTGCCGGCTGCACGGGCCCCAAGCACTCTGACTGCC
TGGCCTGCCTCCACTTCAACCACAGTGGCATCTGTGAGCTGCACTGCCCA
GCCCTGGTCACCTACAACACAGACACGTTTGAGTCCATGCCCAATCCCGA
GGGCCGGTATACATTCGGCGCCAGCTGTGTGACTGCCTGTCCCTACAACT
ACCTTTCTACGGACGTGGGATCCTGCACCCTCGTCTGCCCCCTGCACAAC
CAAGAGGTGACAGCAGAGGAT.

In another embodiment, the nucleic acid sequence encoding the human her2/neu EC2 fragment implemented into the chimera spans from 1077-1554 bp of the human her2/neu EC2 fragment and includes a 50 bp extension, and is set forth in (SEQ ID NO: 51).

(SEQ ID NO: 51)

AATATCCAGGAGTTTGCTGGCTGCAAGAAGATCTTTGGGAGCCTGGCATT
TCTGCCGGAGAGCTTTGATGGGGACCCAGCCTCCAACACTGCCCCGCTCC
AGCCAGAGCAGCTCCAAGTGTTTGAGACTCTGGAAGAGATCACAGGTTAC
CTATACATCTCAGCATGGCCGGACAGCCTGCCTGACCTCAGCGTCTTCCA
GAACCTGCAAGTAATCCGGGGACGAATTCTGCACAATGGCGCCTACTCGC
TGACCCTGCAAGGGCTGGGCATCAGCTGGCTGGGGCTGCGCTCACTGAGG
GAACTGGGCAGTGGACTGGCCCTCATCCACCATAACACCCACCTCTGCTT
CGTGCACACGGTGCCCTGGGACCAGCTCTTTCGGAACCCGCACCAAGCTC
TGCTCCACACTGCCAACCGGCCAGAGGACGAGTGTGTGGGCGAGGGCCTG
GCCTGCCACCAGCTGTGCGCCCGAGGG.

In one embodiment, complete EC2 human her2/neu fragment spans from 907-1504 bp of the human her2/neu gene and is set forth in (SEQ ID NO: 55).

(SEQ ID NO: 55)

TACCTTTCTACGGACGTGGGATCCTGCACCCTCGTCTGCCCCCTGCACAA
CCAAGAGGTGACAGCAGAGGATGGAACACAGCGGTGTGAGAAGTGCAGCA
AGCCCTGTGCCCGAGTGTGCTATGGTCTGGGCATGGAGCACTTGCGAGAG
GTGAGGGCAGTTACCAGTGCCAATATCCAGGAGTTTGCTGGCTGCAAGAA
GATCTTTGGGAGCCTGGCATTTCTGCCGGAGAGCTTTGATGGGGACCCAG
CCTCCAACACTGCCCCGCTCCAGCCAGAGCAGCTCCAAGTGTTTGAGACT
CTGGAAGAGATCACAGGTTACCTATACATCTCAGCATGGCCGGACAGCCT
GCCTGACCTCAGCGTCTTCCAGAACCTGCAAGTAATCCGGGGACGAATTC
TGCACAATGGCGCCTACTCGCTGACCCTGCAAGGGCTGGGCATCAGCTGG
CTGGGGCTGCGCTCACTGAGGGAACTGGGCAGTGGACTGGCCCTCATCCA
CCATAACACCCACCTCTGCTTCGTGCACACGGTGCCCTGGGACCAGCTCT
TTCGGAACCCGCACCAAGCTCTGCTCCACACTGCCAACCGGCCAGAG.

In another embodiment, the nucleic acid sequence encoding the human her2/neu IC1 fragment implemented into the chimera is set forth in (SEQ ID NO: 52).

(SEQ ID NO: 52)

CAGCAGAAGATCCGGAAGTACACGATGCGGAGACTGCTGCAGGAAACGGA
GCTGGTGGAGCCGCTGACACCTAGCGGAGCGATGCCCAACCAGGCGCAGA
TGCGGATCCTGAAAGAGACGGAGCTGAGGAAGGTGAAGGTGCTTGGATCT
GGCGCTTTTGGCACAGTCTACAAGGGCATCTGGATCCCTGATGGGGAGAA
TGTGAAAATTCCAGTGGCCATCAAAGTGTTGAGGGAAAACACATCCCCCA
AAGCCAACAAAGAAATCTTAGACGAAGCATACGTGATGGCTGGTGTGGGC
TCCCCATATGTCTCCCGCCTTCTGGGCATCTGCCTGACATCCACGGTGCA
GCTGGTGACACAGCTTATGCCCTATGGCTGCCTCTTAGACT.

In another embodiment, the nucleic acid sequence encoding the complete human her2/neu IC 1 fragment spans from 2034-3243 of the human her2/neu gene and is set forth in (SEQ ID NO: 56).

(SEQ ID NO: 56)

CAGCAGAAGATCCGGAAGTACACGATGCGGAGACTGCTGCAGGAAACGGA
GCTGGTGGAGCCGCTGACACCTAGCGGAGCGATGCCCAACCAGGCGCAGA
TGCGGATCCTGAAAGAGACGGAGCTGAGGAAGGTGAAGGTGCTTGGATCT
GGCGCTTTTGGCACAGTCTACAAGGGCATCTGGATCCCTGATGGGGAGAA
TGTGAAAATTCCAGTGGCCATCAAAGTGTTGAGGGAAAACACATCCCCCA
AAGCCAACAAAGAAATCTTAGACGAAGCATACGTGATGGCTGGTGTGGGC
TCCCCATATGTCTCCCGCCTTCTGGGCATCTGCCTGACATCCACGGTGCA
GCTGGTGACACAGCTTATGCCCTATGGCTGCCTCTTAGACCATGTCCGGG
AAAACCGCGGACGCCTGGGCTCCCAGGACCTGCTGAACTGGTGTATGCAG
ATTGCCAAGGGGATGAGCTACCTGGAGGATGTGCGGCTCGTACACAGGGA
CTTGGCCGCTCGGAACGTGCTGGTCAAGAGTCCCAACCATGTCAAAATTA
CAGACTTCGGGCTGGCTCGGCTGCTGGACATTGACGAGACAGAGTACCAT
GCAGATGGGGGCAAGGTGCCCATCAAGTGGATGGCGCTGGAGTCCATTCT
CCGCCGGCGGTTCACCCACCAGAGTGATGTGTGGAGTTATGGTGTGACTG
TGTGGGAGCTGATGACTTTTGGGGCCAAACCTTACGATGGGATCCCAGCC
CGGGAGATCCCTGACCTGCTGGAAAAGGGGGAGCGGCTGCCCCAGCCCCC
CATCTGCACCATTGATGTCTACATGATCATGGTCAAATGTTGGATGATTG
ACTCTGAATGTCGGCCAAGATTCCGGGAGTTGGTGTCTGAATTCTCCCGC
ATGGCCAGGGACCCCCAGCGCTTTGTGGTCATCCAGAATGAGGACTTGGG
CCCAGCCAGTCCCTTGGACAGCACCTTCTACCGCTCACTGCTGGAGGACG
ATGACATGGGGGACCTGGTGGATGCTGAGGAGTATCTGGTACCCCAGCAG
GGCTTCTTCTGTCCAGACCCTGCCCCGGGCGCTGGGGGCATGGTCCACCA
CAGGCACCGCAGCTCATCTACCAGGAGTGGCGGTGGGGACCTGACACTAG
GGCTGGAGCCCTCTGAAGAGGAGGCCCCCAGGTCTCCACTGGCACCCTCC
GAAGGGGCT.

The LLO utilized in the methods and compositions provided herein is, in one embodiment, a Listeria LLO. In one embodiment, the Listeria from which the LLO is derived is Listeria monocytogenes (LM). In another embodiment, the Listeria is Listeria ivanovii. In another embodiment, the Listeria is Listeria welshimeri. In another embodiment, the Listeria is Listeria seeligeri. In another embodiment, the LLO protein is a non-Listerial LLO protein. In another embodiment, the LLO protein is a synthetic LLO protein. In another embodiment it is a recombinant LLO protein.

In one embodiment, the LLO protein is encoded by the following nucleic acid sequence set forth in (SEQ ID NO: 3)

(SEQ ID NO: 3)

atgaaaaaaataatgctagtttttattacacttatattagttagtctacc
aattgcgcaacaaactgaagcaaaggatgcatctgcattcaataaagaaa
attcaatttcatccatggcaccaccagcatctccgcctgcaagtcctaag
acgccaatcgaaaagaaacacgcggatgaaatcgataagtatatacaagg
attggattacaataaaaacaatgtattagtataccacggagatgcagtga
caaatgtgccgccaagaaaaggttacaaagatggaaatgaatatattgtt
gtggagaaaaagaagaaatccatcaatcaaaataatgcagacattcaagt
tgtgaatgcaatttcgagcctaacctatccaggtgctctcgtaaaagcga
attcggaattagtagaaaatcaaccagatgttctccctgtaaaacgtgat
tcattaacactcagcattgatttgccaggtatgactaatcaagacaataa
aatagttgtaaaaaatgccactaaatcaaacgttaacaacgcagtaaata
cattagtggaaagatggaatgaaaaatatgctcaagcttatccaaatgta
agtgcaaaaattgattatgatgacgaaatggcttacagtgaatcacaatt
aattgcgaaatttggtacagcatttaaagctgtaaataatagcttgaatg
taaacttcggcgcaatcagtgaagggaaaatgcaagaagaagtcattagt
tttaaacaaatttactataacgtgaatgttaatgaacctacaagaccttc
cagatttttcggcaaagctgttactaaagagcagttgcaagcgcttggag
tgaatgcagaaaatcctcctgcatatatctcaagtgtggcgtatggccgt
caagtttatttgaaattatcaactaattcccatagtactaaagtaaaagc
tgcttttgatgctgccgtaagcggaaaatctgtctcaggtgatgtagaac
taacaaatatcatcaaaaattcttccttcaaagccgtaatttacggaggt
tccgcaaaagatgaagttcaaatcatcgacggcaacctcggagacttacg
cgatattttgaaaaaaggcgctacttttaatcgagaaacaccaggagttc
ccattgcttatacaacaaacttcctaaaagacaatgaattagctgttatt
aaaaacaactcagaatatattgaaacaacttcaaaagcttatacagatgg
aaaaattaacatcgatcactctggaggatacgttgctcaattcaacattt
cttgggatgaagtaaattatgat.

In another embodiment, the LLO protein has the sequence SEQ ID NO: 4

(SEQ ID NO: 4)
M K K I M L V F I T L I L V S L P I A Q Q T E A K D A S A F N K E
N S I S S M A P P A S P P A S P K T P I E K K H A D E I D K Y I Q G
L D Y N K N N V L V Y H G D A V T N V P P R K G Y K D G N E Y I
V V E K K K K S I N Q N N A D I Q V V N A I S S L T Y P G A L V K
A N S E L V E N Q P D V L P V K R D S L T L S I D L P G M T N Q D
N K I V V K N A T K S N V N N A V N T L V E R W N E K Y A Q A Y
P N V S A K I D Y D D E M A Y S E S Q L I A K F G T A F K A V N N
S L N V N F G A I S E G K M Q E E V I S F K Q I Y Y N V N V N E P
T R P S R F F G K A V T K E Q L Q A L G V N A E N P P A Y I S S V
A Y G R Q V Y L K L S T N S H S T K V K A A F D A A V S G K S V
S G D V E L T N I I K N S S F K A V I Y G G S A K D E V Q I I D G
N L G D L R D I L K K G A T F N R E T P G V P I A Y T T N F L K D
N E L A V I K N N S E Y I E T T S K A Y T D G K I N I D H S G G Y
V A Q F N I S W D E V N Y D

The first 25 amino acids of the proprotein corresponding to this sequence are the signal sequence and are cleaved from LLO when it is secreted by the bacterium. Thus, in this embodiment, the full length active LLO protein is 504 residues long. In another embodiment, the LLO protein has a sequence set forth in GenBank Accession No. DQ054588, DQ054589, AY878649, U25452, or U25452. In another embodiment, the LLO protein is a variant of an LLO protein. In another embodiment, the LLO protein is a homologue of an LLO protein. Each possibility represents a separate embodiment of the present invention.

In another embodiment, “truncated LLO” or “tLLO” refers to a fragment of LLO that comprises the PEST-like domain. In another embodiment, the terms refer to an LLO fragment that does not contain the activation domain at the amino terminus and does not include cystine 484. In another embodiment, the LLO fragment consists of a PEST sequence. In another embodiment, the LLO fragment comprises a PEST sequence. In another embodiment, the LLO fragment consists of about the first 400 to 441 amino acids of the 529 amino acid full-length LLO protein. In another embodiment, the LLO fragment is a non-hemolytic form of the LLO protein.

In another embodiment of methods and compositions of the present invention, a polypeptide encoded by a nucleic acid sequence of methods and compositions of the present invention is a fusion protein comprising the chimeric Her-2/neu antigen and an additional polypeptide, where in another embodiment, the fusion protein comprises, inter alia, an LM non-hemolytic LLO protein (Examples herein).

In one embodiment, the LLO fragment consists of about residues 1-25. In another embodiment, the LLO fragment consists of about residues 1-50. In another embodiment, the LLO fragment consists of about residues 1-75. In another embodiment, the LLO fragment consists of about residues 1-100. In another embodiment, the LLO fragment consists of about residues 1-125. In another embodiment, the LLO fragment consists of about residues 1-150. In another embodiment, the LLO fragment consists of about residues 1175. In another embodiment, the LLO fragment consists of about residues 1-200. In another embodiment, the LLO fragment consists of about residues 1-225. In another embodiment, the LLO fragment consists of about residues 1-250. In another embodiment, the LLO fragment consists of about residues 1-275. In another embodiment, the LLO fragment consists of about residues 1-300. In another embodiment, the LLO fragment consists of about residues 1-325. In another embodiment, the LLO fragment consists of about residues 1-350. In another embodiment, the LLO fragment consists of about residues 1-375. In another embodiment, the LLO fragment consists of about residues 1-400. In another embodiment, the LLO fragment consists of about residues 1-425. Each possibility represents a separate embodiment of the present invention.

In another embodiment, a fusion protein of methods and compositions of the present invention comprises a PEST sequence, either from an LLO protein or from another organism, e.g. a prokaryotic organism.

The PEST-like AA sequence has, in another embodiment, a sequence selected from SEQ ID NO: 5-9. In another embodiment, the PEST-like sequence is a PEST-like sequence from the LM ActA protein. In another embodiment, the PEST-like sequence is KTEEQPSEVNTGPR (SEQ ID NO: 5), KASVTDTSEGDLDSSMQSADESTPQPLK (SEQ ID NO: 6), KNEEVNASDFPPPPTDEELR (SEQ ID NO: 7), or RGGIPTSEEFSSLNSGDFTDDENSETTEEEIDR (SEQ ID NO: 8). In another embodiment, the PEST-like sequence is from Streptolysin O protein of Streptococcus sp. In another embodiment, the PEST-like sequence is from Streptococcus pyogenes Streptolysin O, e.g. KQNTASTETTTTNEQPK (SEQ ID NO: 9) at AA 35-51. In another embodiment, the PEST-like sequence is from Streptococcus equisimilis Streptolysin O, e.g. KQNTANTETTTTNEQPK (SEQ ID NO: 10) at AA 38-54. In another embodiment, the PEST-like sequence is another PEST-like AA sequence derived from a prokaryotic organism. In another embodiment, the PEST-like sequence is any other PEST-like sequence known in the art. Each possibility represents a separate embodiment of the present invention.

In one embodiment, fusion of an antigen to the PEST-like sequence of LM enhanced cell mediated and anti-tumor immunity of the antigen. Thus, fusion of an antigen to other PEST-like sequences derived from other prokaryotic organisms will also enhance immunogenicity of the antigen. PEST-like sequence of other prokaryotic organism can be identified in accordance with methods such as described by, for example Rechsteiner and Rogers (1996, Trends Biochem. Sci. 21:267-271) for LM. Alternatively, PEST-like AA sequences from other prokaryotic organisms can also be identified based by this method. Other prokaryotic organisms wherein PEST-like AA sequences would be expected to include, but are not limited to, other Listeria species. In another embodiment, the PEST-like sequence is embedded within the antigenic protein. Thus, in another embodiment, “fusion” refers to an antigenic protein comprising both the antigen and the PEST-like amino acid sequence either linked at one end of the antigen or embedded within the antigen.

In another embodiment, provided herein is a vaccine comprising a recombinant polypeptide of the present invention. In another embodiment, provided herein is a vaccine consisting of a recombinant polypeptide of the present invention.

In another embodiment, provided herein is a nucleotide molecule encoding a recombinant polypeptide of the present invention. In another embodiment, provided herein is a vaccine comprising the nucleotide molecule.

In another embodiment, provided herein is a nucleotide molecule encoding a recombinant polypeptide of the present invention.

In another embodiment, provided herein is a recombinant polypeptide encoded by the nucleotide molecule of the present invention.

In another embodiment, provided herein is a vaccine comprising a nucleotide molecule or recombinant polypeptide of the present invention.

In another embodiment, provided herein is an immunogenic composition comprising a nucleotide molecule or recombinant polypeptide of the present invention.

In another embodiment, provided herein is a vector comprising a nucleotide molecule or recombinant polypeptide of the present invention.

In another embodiment, provided herein is a recombinant form of Listeria comprising a nucleotide molecule of the present invention.

In another embodiment, provided herein is a vaccine comprising a recombinant form of Listeria of the present invention.

In another embodiment, provided herein is a culture of a recombinant form of Listeria of the present invention.

In one embodiment, the vaccine for use in the methods of the present invention comprises a recombinant Listeria monocytogenes, in any form or embodiment as described herein. In one embodiment, the vaccine for use in the present invention consists of a recombinant Listeria monocytogenes of the present invention, in any form or embodiment as described herein. In another embodiment, the vaccine for use in the methods of the present invention consists essentially of a recombinant Listeria monocytogenes of the present invention, in any form or embodiment as described herein. In one embodiment, the term “comprise” refers to the inclusion of a recombinant Listeria monocytogenes in the vaccine, as well as inclusion of other vaccines or treatments that may be known in the art. In another embodiment, the term “consisting essentially of” refers to a vaccine, whose functional component is the recombinant Listeria monocytogenes, however, other components of the vaccine may be included that are not involved directly in the therapeutic effect of the vaccine and may, for example, refer to components which facilitate the effect of the recombinant Listeria monocytogenes (e.g. stabilizing, preserving, etc.). In another embodiment, the term “consisting” refers to a vaccine, which contains the recombinant Listeria monocytogenes.

In another embodiment, the methods of the present invention comprise the step of administering a recombinant Listeria monocytogenes, in any form or embodiment as described herein. In one embodiment, the methods of the present invention consist of the step of administering a recombinant Listeria monocytogenes of the present invention, in any form or embodiment as described herein. In another embodiment, the methods of the present invention consist essentially of the step of administering a recombinant Listeria monocytogenes of the present invention, in any form or embodiment as described herein. In one embodiment, the term “comprise” refers to the inclusion of the step of administering a recombinant Listeria monocytogenes in the methods, as well as inclusion of other methods or treatments that may be known in the art. In another embodiment, the term “consisting essentially of” refers to a methods, whose functional component is the administration of recombinant Listeria monocytogenes, however, other steps of the methods may be included that are not involved directly in the therapeutic effect of the methods and may, for example, refer to steps which facilitate the effect of the administration of recombinant Listeria monocytogenes. In one embodiment, the term “consisting” refers to a method of administering recombinant Listeria monocytogenes with no additional steps.

In another embodiment, the Listeria of methods and compositions of the present invention is Listeria monocytogenes. In another embodiment, the Listeria is Listeria ivanovii. In another embodiment, the Listeria is Listeria welshimeri. In another embodiment, the Listeria is Listeria seeligeri. Each type of Listeria represents a separate embodiment of the present invention.

In one embodiment, the Listeria strain of the methods and compositions of the present invention is the ADXS31-164 strain. In another embodiment, ADXS31-164 stimulates the secretion of IFN-γ by the splenocytes from wild type FVB/N mice. Further, the data presented herein show that ADXS31-164 is able to elicit anti-Her2/neu specific immune responses to human epitopes that are located at different domains of the targeted antigen.

In another embodiment, the present invention provides a recombinant form of Listeria comprising a nucleotide molecule encoding a Her-2 chimeric protein or a fragment thereof.

In one embodiment, the present invention provides a method of inducing an anti-Her-2 immune response in a subject, comprising administering to the subject a recombinant polypeptide comprising an N-terminal fragment of a LLO protein fused to a Her-2 chimeric protein or fused to a fragment thereof, thereby inducing an anti-Her-2 immune response in a subject.

In one embodiment, the two molecules of the fusion protein (the LLO, ActA fragment or PEST sequence and the antigen) are joined directly. In another embodiment, the two molecules are joined by a short spacer peptide, consisting of one or more amino acids. In one embodiment, the spacer has no specific biological activity other than to join the proteins or to preserve some minimum distance or other spatial relationship between them. In another embodiment, the constituent amino acids of the spacer are selected to influence some property of the molecule such as the folding, net charge, or hydrophobicity. In another embodiment, the two molecules of the protein (the LLO fragment and the antigen) are synthesized separately or unfused. In another embodiment, the two molecules of the protein are synthesized separately from the same nucleic acid. In yet another embodiment, the two molecules are individually synthesized from separate nucleic acids. Each possibility represents a separate embodiment of the present invention.

In one embodiment, nucleic acids encoding the recombinant polypeptides provided herein also encode a signal peptide or sequence. In another embodiment, the fusion protein of methods and compositions of the present invention comprises an LLO signal sequence from LLO. In one embodiment, a heterologous antigen may be expressed through the use of a signal sequence, such as a Listerial signal sequence, for example, the hemolysin signal sequence or the actA signal sequence. Alternatively, for example, foreign genes can be expressed downstream from a L. monocytogenes promoter without creating a fusion protein. In another embodiment, the signal peptide is bacterial (Listerial or non-Listerial). In one embodiment, the signal peptide is native to the bacterium. In another embodiment, the signal peptide is foreign to the bacterium. In another embodiment, the signal peptide is a signal peptide from Listeria monocytogenes, such as a secA 1 signal peptide. In another embodiment, the signal peptide is a Usp45 signal peptide from Lactococcus lactis, or a Protective Antigen signal peptide from Bacillus anthracis. In another embodiment, the signal peptide is a secA2 signal peptide, such the p60 signal peptide from Listeria monocytogenes. In addition, the recombinant nucleic acid molecule optionally comprises a third polynucleotide sequence encoding p60, or a fragment thereof. In another embodiment, the signal peptide is a Tat signal peptide, such as a B. subtilis Tat signal peptide (e.g., PhoD). In one embodiment, the signal peptide is in the same translational reading frame encoding the recombinant polypeptide.

In another embodiment, provided herein is a method of inducing an anti-Her-2 immune response in a subject, comprising administering to the subject a recombinant nucleotide encoding a recombinant polypeptide comprising an N-terminal fragment of a LLO protein fused to a Her-2 chimeric protein or fused to a fragment thereof, thereby inducing an anti-Her-2 immune response in a subject.

In one embodiment, provided herein is a method of eliciting an enhanced immune response to a Her2/neu-expressing tumor in a subject, where in another embodiment the method comprises administering to the subject a composition comprising the recombinant Listeria vaccine strain provided herein. In another embodiment, the immune response against the Her-2-expressing tumor comprises an immune response to a subdominant epitope of the Her-2 protein. In another embodiment, the immune response against the Her-2-expressing tumor comprises an immune response to several subdominant epitopes of the Her-2 protein. In another embodiment, the immune response against the Her-2-expressing tumor comprises an immune response to at least 1-5 subdominant epitopes of the Her-2 protein. In another embodiment, the immune response against the Her-2-expressing tumor comprises an immune response to at least 1-10 subdominant epitopes of the Her-2 protein. In another embodiment, the immune response against the Her-2-expressing tumor comprises an immune response to at least 1-17 subdominant epitopes of the Her-2 protein. In another embodiment, the immune response against the Her-2-expressing tumor comprises an immune response to at least 17 subdominant epitopes of the Her-2 protein.

Point mutations or amino-acid deletions in the oncogenic protein Her2/neu, have been reported to mediate treatment of resistant tumor cells, when these tumors have been targeted by small fragment Listeria-based vaccines or trastuzumab (a monoclonal antibody against an epitope located at the extracellular domain of the Her2/neu antigen). Described herein is a chimeric Her2/neu based composition which harbors two of the extracellular and one intracellular fragments of Her2/neu antigen showing clusters of MHC-class I epitopes of the oncogene. This chimeric protein, which harbors 3 H2Dq and at least 17 of the mapped human MHC-class I epitopes of the Her2/neu antigen was fused to the first 441 amino acids of the Listeria-monocytogenes listeriolysin O protein and expressed and secreted by the Listeria monocytogenes attenuated strain LmddA.

Previous reports have shown that when Her2/neu transgenic mice were immunized with Listeria-based vaccines expressing and secreting small fragments of the Her2/neu antigen separately (each of which harbored only one H2Dq epitope of the Her2/neu oncogene), Her2/neu over-expressing tumors could escape due to mutations in those epitopes of the Her2/neu antigen targeted by each vaccine (see Singh R, Paterson Y. Immunoediting sculpts tumor epitopes during immunotherapy. Cancer Res 2007; 67: 1887-92). Demonstrated herein is the unexpected result that when three or more epitopes of the Her2/neu protein are incorporated in a chimeric vaccine, it can eliminate the selection and escape of these tumors by escape mutations. Immunization with the novel Her2/neu chimeric Listeria vaccines did not result in any escape mutations that could be associated with point mutations or amino acid deletions in the Her2/neu antigen (see Example 4 herein).

In one embodiment, provided herein is a method of engineering a Listeria vaccine strain to express a Her-2 chimeric protein or recombinant polypeptide expressing the chimeric protein, the method comprising transforming a Listeria strain with a nucleic acid molecule. In another embodiment, the nucleic acid molecule comprises a first open reading frame encoding a polypeptide, wherein the polypeptide comprises a Her2/neu chimeric antigen. In another embodiment, the nucleic acid molecule further comprises a second open reading frame encoding a metabolic enzyme, and wherein said metabolic enzyme complements an endogenous gene that is lacking in the chromosome of the recombinant Listeria strain, thereby engineering a Listeria vaccine strain to express a Her-2 chimeric protein.

In one embodiment, the methods and compositions provided herein further comprise an adjuvant, where in another embodiment, the adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

In one embodiment, attenuated Listeria strains, such as LM delta-actA mutant (Brundage et al, 1993, Proc. Natl. Acad. Sci., USA, 90:11890-11894), L. monocytogenes delta-plcA (Camilli et al, 1991, J. Exp. Med., 173:751-754), or delta-ActA, delta INL-b (Brockstedt et 5 al, 2004, PNAS, 101:13832-13837) are used in the present invention. In another embodiment, attenuated Listeria strains are constructed by introducing one or more attenuating mutations, as will be understood by one of average skill in the art when equipped with the disclosure herein. Examples of such strains include, but are not limited to Listeria strains auxotrophic for aromatic amino acids (Alexander et al, 1993, Infection and Immunity 10 61:2245-2248) and mutant for the formation of lipoteichoic acids (Abachin et al, 2002, Mol. Microbiol. 43:1-14) and those attenuated by a lack of a virulence gene (see examples herein).

In another embodiment, the nucleic acid molecule of methods and compositions of the present invention is operably linked to a promoter/regulatory sequence. In another embodiment, the first open reading frame of methods and compositions of the present invention is operably linked to a promoter/regulatory sequence. In another embodiment, the second open reading frame of methods and compositions of the present invention is operably linked to a promoter/regulatory sequence. In another embodiment, each of the open reading frames are operably linked to a promoter/regulatory sequence. Each possibility represents a separate embodiment of the present invention.

The skilled artisan, when equipped with the present disclosure and the methods provided herein, will readily understand that different transcriptional promoters, terminators, carrier vectors or specific gene sequences (e.g. those in commercially available cloning vectors) can be used successfully in methods and compositions of the present invention. As is contemplated in the present invention, these functionalities are provided in, for example, the commercially available vectors known as the pUC series. In another embodiment, non-essential DNA sequences (e.g. antibiotic resistance genes) are removed. Each possibility represents a separate embodiment of the present invention. In another embodiment, a commercially available plasmid is used in the present invention. Such plasmids are available from a variety of sources, for example, Invitrogen (La Jolla, Calif.), Stratagene (La Jolla, Calif.), Clontech (Palo Alto, Calif.), or can be constructed using methods well known in the art.

Another embodiment is a plasmid such as pCR2.1 (Invitrogen, La Jolla, Calif.), which is a prokaryotic expression vector with a prokaryotic origin of replication and promoter/regulatory elements to facilitate expression in a prokaryotic organism. In another embodiment, extraneous nucleotide sequences are removed to decrease the size of the plasmid and increase the size of the cassette that can be placed therein.

Such methods are well known in the art, and are described in, for example, Sambrook et al. (1989, Molecular Cloning: A Laboratory Manual, Cold Spring Harbor Laboratory Press, New York) and Ausubei et al. (1997, Current Protocols in Molecular Biology, Green & Wiley, New York).

Antibiotic resistance genes are used in the conventional selection and cloning processes commonly employed in molecular biology and vaccine preparation. Antibiotic resistance genes contemplated in the present invention include, but are not limited to, gene products that confer resistance to ampicillin, penicillin, methicillin, streptomycin, erythromycin, kanamycin, tetracycline, cloramphenicol (CAT), neomycin, hygromycin, gentamicin and others well known in the art. Each gene represents a separate embodiment of the present invention.

Methods for transforming bacteria are well known in the art, and include calcium-chloride competent cell-based methods, electroporation methods, bacteriophage-mediated transduction, chemical, and physical transformation techniques (de Boer et al, 1989, Cell 56:641-649; Miller et al, 1995, FASEB J., 9:190-199; Sambrook et al. 1989, Molecular Cloning: A Laboratory Manual, Cold Spring Harbor Laboratory, New York; Ausubel et al., 1997, Current Protocols in Molecular Biology, John Wiley & Sons, New York; Gerhardt et al., eds., 1994, Methods for General and Molecular Bacteriology, American Society for Microbiology, Washington, D.C.; Miller, 1992, A Short Course in Bacterial Genetics, Cold Spring Harbor Laboratory Press, Cold Spring Harbor, N.Y.) In another embodiment, the Listeria vaccine strain of the present invention is transformed by electroporation. Each method represents a separate embodiment of the present invention.

In another embodiment, conjugation is used to introduce genetic material and/or plasmids into bacteria. Methods for conjugation are well known in the art, and are described, for example, in Nikodinovic J et al. (A second generation snp-derived Escherichia coli-Streptomyces shuttle expression vector that is generally transferable by conjugation. Plasmid. 2006 November; 56(3):223-7) and Auchtung J M et al (Regulation of a Bacillus subtilis mobile genetic element by intercellular signaling and the global DNA damage response. Proc Natl Acad Sci USA. 2005 Aug. 30; 102 (35):12554-9). Each method represents a separate embodiment of the present invention.

“Transforming,” in one embodiment, is used identically with the term “transfecting,” and refers to engineering a bacterial cell to take up a plasmid or other heterologous DNA molecule. In another embodiment, “transforming” refers to engineering a bacterial cell to express a gene of a plasmid or other heterologous DNA molecule. Each possibility represents a separate embodiment of the present invention.

Plasmids and other expression vectors useful in the present invention are described elsewhere herein, and can include such features as a promoter/regulatory sequence, an origin of replication for gram negative and gram positive bacteria, an isolated nucleic acid encoding a fusion protein and an isolated nucleic acid encoding an amino acid metabolism gene. Further, an isolated nucleic acid encoding a fusion protein and an amino acid metabolism gene will have a promoter suitable for driving expression of such an isolated nucleic acid. Promoters useful for driving expression in a bacterial system are well known in the art, and include bacteriophage lambda, the bla promoter of the beta-lactamase gene of pBR322, and the CAT promoter of the chloramphenicol acetyl transferase gene of pBR325. Further examples of prokaryotic promoters include the major right and left promoters of 5 bacteriophage lambda (PL and PR), the trp, recA, lacZ, lad, and gal promoters of E. coli, the alpha-amylase (Ulmanen et al, 1985. J. Bacteriol. 162:176-182) and the S28-specific promoters of B. subtilis (Gilman et al, 1984 Gene 32:11-20), the promoters of the bacteriophages of Bacillus (Gryczan, 1982, In: The Molecular Biology of the Bacilli, Academic Press, Inc., New York), and Streptomyces promoters (Ward et al, 1986, Mol. Gen. Genet. 203:468-478). Additional prokaryotic promoters contemplated in the present invention are reviewed in, for example, Glick (1987, J. Ind. Microbiol. 1:277-282); Cenatiempo, (1986, Biochimie, 68:505-516); and Gottesman, (1984, Ann. Rev. Genet. 18:415-442). Further examples of promoter/regulatory elements contemplated in the present invention include, but are not limited to the Listerial prfA promoter, the Listerial hly promoter, the Listerial p60 promoter and the Listerial ActA promoter (GenBank Acc. No. NC_{—}003210) or fragments thereof.

In another embodiment, a plasmid of methods and compositions of the present invention comprises a gene encoding a fusion protein. In another embodiment, subsequences are cloned and the appropriate subsequences cleaved using appropriate restriction enzymes. The fragments are then, in another embodiment, ligated to produce the desired DNA sequence. In another embodiment, DNA encoding the antigen is produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The antigen is ligated into a plasmid. Each method represents a separate embodiment of the present invention.

In another embodiment, the present invention further comprises a phage based chromosomal integration system for clinical applications. A host strain that is auxotrophic for essential enzymes, including, but not limited to, d-alanine racemase will be used, for example Lmdal(−)dat(−). In another embodiment, in order to avoid a “phage curing step,” a phage integration system based on PSA is used (Lauer, et al., 2002 J Bacteriol, 184:4177-4186). This requires, in another embodiment, continuous selection by antibiotics to maintain the integrated gene. Thus, in another embodiment, the current invention enables the establishment of a phage based chromosomal integration system that does not require selection with antibiotics. Instead, an auxotrophic host strain will be complemented.

The recombinant proteins of the present invention are synthesized, in another embodiment, using recombinant DNA methodology. This involves, in one embodiment, creating a DNA sequence that encodes the fusion protein, placing the DNA in an expression cassette, such as the plasmid of the present invention, under the control of a particular promoter/regulatory element, and expressing the protein. DNA encoding the fusion protein (e.g. non-hemolytic LLO/antigen) of the present invention is prepared, in another embodiment, by any suitable method, including, for example, cloning and restriction of appropriate sequences or direct chemical synthesis by methods such as the phosphotriester method of Narang et al. (1979, Meth. Enzymol. 68: 90-99); the phosphodiester method of Brown et al. (1979, Meth. Enzymol 68: 109-151); the diethylphosphoramidite method of Beaucage et al. (1981, Tetra. Lett., 22: 15 1859-1862); and the solid support method of U.S. Pat. No. 4,458,066.

In another embodiment, chemical synthesis is used to produce a single stranded oligonucleotide. This single stranded oligonucleotide is converted, in various embodiments, into double stranded DNA by hybridization with a complementary sequence, or by polymerization with a DNA polymerase using the single strand as a template. One of skill in the art would recognize that while chemical synthesis of DNA is limited to sequences of about 100 bases, longer sequences can be obtained by the ligation of shorter sequences. In another embodiment, subsequences are cloned and the appropriate subsequences cleaved using appropriate restriction enzymes. The fragments are then ligated to produce the desired DNA sequence.

In another embodiment, DNA encoding the fusion protein or the recombinant protein of the present invention is cloned using DNA amplification methods such as polymerase chain reaction (PCR). Thus, the gene for non-hemolytic LLO is PCR amplified, using a sense primer comprising a suitable restriction site and an antisense primer comprising another restriction site, e.g. a non-identical restriction site to facilitate cloning. The same is repeated for the isolated nucleic acid encoding an antigen. Ligation of the non-hemolytic LLO and antigen sequences and insertion into a plasmid or vector produces a vector encoding non-hemolytic LLO joined to a terminus of the antigen. The two molecules are joined either directly or by a short spacer introduced by the restriction site.

In another embodiment, the molecules are separated by a peptide spacer consisting of one or more amino acids, generally the spacer will have no specific biological activity other than to join the proteins or to preserve some minimum distance or other spatial relationship between them. In another embodiment, the constituent AA of the spacer are selected to influence some property of the molecule such as the folding, net charge, or hydrophobicity. In another embodiment, the nucleic acid sequences encoding the fusion or recombinant proteins are transformed into a variety of host cells, including E. coli, other bacterial hosts, such as Listeria, yeast, and various higher eukaryotic cells such as the COS, CHO and HeLa cells lines and myeloma cell lines. The recombinant fusion protein gene will be operably linked to appropriate expression control sequences for each host. Promoter/regulatory sequences are described in detail elsewhere herein. In another embodiment, the plasmid further comprises additional promoter regulatory elements, as well as a ribosome binding site and a transcription termination signal. For eukaryotic cells, the control sequences will include a promoter and an enhancer derived from e.g. immunoglobulin genes, SV40, cytomegalovirus, etc., and a polyadenylation sequence. In another embodiment, the sequences include splice donor and acceptor sequences.

In one embodiment, the term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

In another embodiment, in order to select for an auxotrophic bacterium comprising the plasmid, transformed auxotrophic bacteria are grown on a media that will select for expression of the amino acid metabolism gene. In another embodiment, a bacteria auxotrophic for D-glutamic acid synthesis is transformed with a plasmid comprising a gene for D-glutamic acid synthesis, and the auxotrophic bacteria will grow in the absence of D-glutamic acid, whereas auxotrophic bacteria that have not been transformed with the plasmid, or are not expressing the plasmid encoding a protein for D-glutamic acid synthesis, will not grow. In another embodiment, a bacterium auxotrophic for D-alanine synthesis will grow in the absence of D-alanine when transformed and expressing the plasmid of the present invention if the plasmid comprises an isolated nucleic acid encoding an amino acid metabolism enzyme for D-alanine synthesis. Such methods for making appropriate media comprising or lacking necessary growth factors, supplements, amino acids, vitamins, antibiotics, and the like are well known in the art, and are available commercially (Becton-Dickinson, Franklin Lakes, N.J.). Each method represents a separate embodiment of the present invention.

In another embodiment, once the auxotrophic bacteria comprising the plasmid of the present invention have been selected on appropriate media, the bacteria are propagated in the presence of a selective pressure. Such propagation comprises growing the bacteria in media without the auxotrophic factor. The presence of the plasmid expressing an amino acid metabolism enzyme in the auxotrophic bacteria ensures that the plasmid will replicate along with the bacteria, thus continually selecting for bacteria harboring the plasmid. The skilled artisan, when equipped with the present disclosure and methods herein will be readily able to scale-up the production of the Listeria vaccine vector by adjusting the volume of the media in which the auxotrophic bacteria comprising the plasmid are growing.

The skilled artisan will appreciate that, in another embodiment, other auxotroph strains and complementation systems are adopted for the use with this invention.

In one embodiment, provided herein is a method of impeding a growth of a Her-2-expressing tumor in a subject, wherein and in another embodiment, the method comprises the step of administering to the subject a composition comprising the recombinant Listeria vaccine strain described herein.

In another embodiment, provided herein is a method of impeding a growth of a Her-2-expressing tumor in a subject, wherein and in another embodiment, the method comprises the step of administering to the subject a composition comprising the recombinant Listeria vaccine strain described herein.

In another embodiment, provided herein is a method of eliciting an enhanced immune response to a Her2/neu-expressing tumor in a subject, wherein and in another embodiment, the method comprises the step of administering to the subject a composition comprising the recombinant Listeria vaccine strain described herein. In yet another embodiment, the immune response against the Her2/neu-expressing tumor comprises an immune response to at least one subdominant epitope of the Her2/neu protein.

In one embodiment, provided herein is a method of preventing an escape mutation in the treatment of Her2/neu over-expressing tumors, wherein and in another embodiment, the method comprises the step of administering to said subject a composition comprising the recombinant Listeria vaccine strain provided herein.

In another embodiment, provided herein is a method of preventing the onset of a Her2/neu antigen-expressing tumor in a subject, wherein and in another embodiment, the method comprises the step of administering to the subject a composition comprising the recombinant Listeria vaccine strain provided herein.

In one embodiment, provided herein is a method of decreasing the frequency of intra-tumoral T regulatory cells, wherein and in another embodiment, the method comprises the step of administering to the subject a composition comprising the recombinant Listeria vaccine strain provided herein.

In another embodiment, provided herein is a method of decreasing the frequency of intra-tumoral T regulatory cells, wherein and in another embodiment, the method comprises the step of administering to the subject a composition comprising the recombinant Listeria vaccine strain provided herein.

In one embodiment, provided herein is a method of decreasing the frequency of intra-tumoral myeloid derived suppressor cells, wherein and in another embodiment, the method comprises the step of administering to the subject a composition comprising the recombinant Listeria vaccine strain provided herein.

In another embodiment, provided herein is a method of decreasing the frequency of myeloid derived suppressor cells, wherein and in another embodiment, the method comprises the step of administering to the subject a composition comprising the recombinant Listeria vaccine strain provided herein.

In one embodiment, provided herein a method of preventing the formation of a Her2/neu-expressing tumor in a subject, wherein and in another embodiment, the method comprises the step of administering to the subject a composition comprising the recombinant Listeria vaccine strain provided herein.

In another embodiment, provided herein is a method of preventing the formation of a Her2/neu-expressing tumor in a subject, wherein and in another embodiment, the method comprises the step of administering to the subject a composition comprising the recombinant Listeria vaccine strain the provided herein.

In one embodiment, provided herein is a method of treating a Her2/neu-expressing tumor in a subject, wherein and in another embodiment, the method comprises the step of administering to the subject a composition comprising the recombinant Listeria vaccine strain provided herein.

In one embodiment, provided herein is a method of administering the composition of the present invention. In another embodiment, provided herein is a method of administering the vaccine of the present invention. In another embodiment, provided herein is a method of administering the recombinant polypeptide or recombinant nucleotide of the present invention. In another embodiment, the step of administering the composition, vaccine, recombinant polypeptide or recombinant nucleotide of the present invention is performed with an attenuated recombinant form of Listeria comprising the composition, vaccine, recombinant nucleotide or expressing the recombinant polypeptide, each in its own discrete embodiment. In another embodiment, the administering is performed with a different attenuated bacterial vector. In another embodiment, the administering is performed with a DNA vaccine (e.g. a naked DNA vaccine). In another embodiment, administration of a recombinant polypeptide of the present invention is performed by producing the protein recombinantly, then administering the recombinant protein to a subject. Each possibility represents a separate embodiment of the present invention.

In another embodiment, the immune response elicited by methods and compositions of the present invention comprises a CD8⁺ T cell-mediated response. In another embodiment, the immune response consists primarily of a CD8⁺ T cell-mediated response. In another embodiment, the only detectable component of the immune response is a CD8⁺ T cell-mediated response.

In another embodiment, the immune response elicited by methods and compositions provided herein comprises a CD4⁺ T cell-mediated response. In another embodiment, the immune response consists primarily of a CD4⁺ T cell-mediated response. In another embodiment, the only detectable component of the immune response is a CD4⁺ T cell-mediated response. In another embodiment, the CD4⁺ T cell-mediated response is accompanied by a measurable antibody response against the antigen. In another embodiment, the CD4⁺ T cell-mediated response is not accompanied by a measurable antibody response against the antigen.

In another embodiment, the present invention provides a method of inducing a CD8⁺ T cell-mediated immune response in a subject against a subdominant CD8⁺ T cell epitope of an antigen, comprising the steps of (a) fusing a nucleotide molecule encoding the Her2-neu chimeric antigen or a fragment thereof to a nucleotide molecule encoding an N-terminal fragment of a LLO protein, thereby creating a recombinant nucleotide encoding an LLO-antigen fusion protein; and (b) administering the recombinant nucleotide or the LLO-antigen fusion to the subject; thereby inducing a CD8⁺ T cell-mediated immune response against a subdominant CD8⁺ T cell epitope of an antigen.

In one embodiment, provided herein is a method of increasing intratumoral ratio of CD8+/T regulatory cells, wherein and in another embodiment, the method comprises the step of administering to the subject a composition comprising the recombinant polypeptide, recombinant Listeria, or recombinant vector of the present invention.

In another embodiment, provided herein is a method of increasing intratumoral ratio of CD8+/T regulatory cells, wherein and in another embodiment, the method comprises the step of administering to the subject a composition comprising the recombinant polypeptide, recombinant Listeria, or recombinant vector of the present invention.

In another embodiment, the immune response elicited by the methods and compositions provided herein comprises an immune response to at least one subdominant epitope of the antigen. In another embodiment, the immune response does not comprise an immune response to a subdominant epitope. In another embodiment, the immune response consists primarily of an immune response to at least one subdominant epitope. In another embodiment, the only measurable component of the immune response is an immune response to at least one subdominant epitope. Each type of immune response represents a separate embodiment of the present invention.

Methods of measuring immune responses are well known in the art, and include, e.g. measuring suppression of tumor growth, flow cytometry, target cell lysis assays (e.g. chromium release assay), the use of tetramers, and others. Each method represents a separate embodiment of the present invention.

In another embodiment, the present invention provides a method of impeding a growth of a Her-2-expressing tumor in a subject, wherein and in another embodiment, the method comprises administering to the subject a recombinant polypeptide comprising an N-terminal fragment of a LLO protein fused to the Her-2 chimeric protein or a fragment thereof or a recombinant nucleotide encoding the recombinant polypeptide, wherein the subject mounts an immune response against the Her-2-expressing tumor, thereby impeding a growth of a Her-2-expressing tumor in a subject.

In another embodiment, the present invention provides a method of improving an antigenicity of a Her-2 chimeric protein, wherein and in another embodiment, the method comprises the step of fusing a nucleotide encoding an N-terminal fragment of a LLO protein to a nucleotide encoding the Her-2 protein or a fragment thereof to create a recombinant nucleotide, thereby improving an antigenicity of a Her-2 chimeric protein.

In another embodiment, provided herein is a method of improving an antigenicity of a Her-2 chimeric protein, wherein and in another embodiment, the method comprises engineering a Listeria strain to express the recombinant nucleotide. In another embodiment, a different bacterial vector is used to express the recombinant nucleotide. In another embodiment, the bacterial vector is attenuated. In another embodiment, a DNA vaccine (e.g. a naked DNA vaccine) is used to express the recombinant nucleotide. In another embodiment, administration of the LLO-Her-2 chimera fusion peptide encoded by the nucleotide is performed by producing the protein recombinantly, then administering the recombinant protein to a subject. Each possibility represents a separate embodiment of the present invention.

In one embodiment, the present invention provides a method for “epitope spreading” of a tumor. In another embodiment, the immunization using the compositions and methods provided herein induce epitope spreading onto other tumors bearing antigens other than the antigen carried in the vaccine of the present invention.

In another embodiment, the dominant epitope or subdominant epitope is dominant or subdominant, respectively, in the subject being treated. In another embodiment, the dominant epitope or subdominant epitope is dominant or subdominant in a population being treated.

In one embodiment, provided herein is a method of treating, suppressing, or inhibiting a cancer or a tumor growth in a subject by epitope spreading wherein and in another embodiment, said cancer is associated with expression of an antigen or fragment thereof comprised in the composition of the present invention. In another embodiment, the method comprises administering to said subject a composition comprising the recombinant polypeptide, recombinant Listeria, or recombinant vector of the present invention. In yet another embodiment, the subject mounts an immune response against the antigen-expressing cancer or the antigen-expressing tumor, thereby treating, suppressing, or inhibiting a cancer or a tumor growth in a subject.

“Dominant CD8⁺ T cell epitope,” in one embodiment, refers to an epitope that is recognized by over 30% of the antigen-specific CD8⁺ T cells that are elicited by vaccination, infection, or a malignant growth with a protein or a pathogen or cancer cell containing the protein. In another embodiment, the term refers to an epitope recognized by over 35% of the antigen-specific CD8⁺ T cells that are elicited thereby. In another embodiment, the term refers to an epitope recognized by over 40% of the antigen-specific CD8⁺ T cells. In another embodiment, the term refers to an epitope recognized by over 45% of the antigen-specific CD8⁺ T cells. In another embodiment, the term refers to an epitope recognized by over 50% of the antigen-specific CD8⁺ T cells. In another embodiment, the term refers to an epitope recognized by over 55% of the antigen-specific CD8⁺ T cells. In another embodiment, the term refers to an epitope recognized by over 60% of the antigen-specific CD8⁺ T cells. In another embodiment, the term refers to an epitope recognized by over 65% of the antigen-specific CD8⁺ T cells. In another embodiment, the term refers to an epitope recognized by over 70% of the antigen-specific CD8⁺ T cells. In another embodiment, the term refers to an epitope recognized by over 75% of the antigen-specific CD8⁺ T cells. In another embodiment, the term refers to an epitope recognized by over 80% of the antigen-specific CD8⁺ T cells. In another embodiment, the term refers to an epitope recognized by over 85% of the antigen-specific CD8⁺ T cells. In another embodiment, the term refers to an epitope recognized by over 90% of the antigen-specific CD8⁺ T cells. In another embodiment, the term refers to an epitope recognized by over 95% of the antigen-specific CD8⁺ T cells. In another embodiment, the term refers to an epitope recognized by over 96% of the antigen-specific CD8⁺ T cells. In another embodiment, the term refers to an epitope recognized by over 97% of the antigen-specific CD8⁺ T cells. In another embodiment, the term refers to an epitope recognized by over 98% of the antigen-specific CD8⁺ T cells.

“Subdominant CD8⁺ T cell epitope,” in one embodiment, refers to an epitope recognized by fewer than 30% of the antigen-specific CD8⁺ T cells that are elicited by vaccination, infection, or a malignant growth with a protein or a pathogen or cancer cell containing the protein. In another embodiment, the term refers to an epitope recognized by fewer than 28% of the antigen-specific CD8⁺ T cells. In another embodiment, the term refers to an epitope recognized by over 26% of the antigen-specific CD8⁺ T cells. In another embodiment, the term refers to an epitope recognized by fewer than 24% of the antigen-specific CD8⁺ T cells. In another embodiment, the term refers to an epitope recognized by over 22% of the antigen-specific CD8⁺ T cells. In another embodiment, the term refers to an epitope recognized by fewer than 20% of the antigen-specific CD8⁺ T cells. In another embodiment, the term refers to an epitope recognized by over 18% of the antigen-specific CD8⁺ T cells. In another embodiment, the term refers to an epitope recognized by fewer than 16% of the antigen-specific CD8⁺ T cells. In another embodiment, the term refers to an epitope recognized by over 14% of the antigen-specific CD8⁺ T cells. In another embodiment, the term refers to an epitope recognized by over 12% of the antigen-specific CD8⁺ T cells. In another embodiment, the term refers to an epitope recognized by fewer than 10% of the antigen-specific CD8⁺ T cells. In another embodiment, the term refers to an epitope recognized by over 8% of the antigen-specific CD8⁺ T cells. In another embodiment, the term refers to an epitope recognized by fewer than 6% of the antigen-specific CD8⁺ T cells. In another embodiment, the term refers to an epitope recognized by fewer than 5% of the antigen-specific CD8⁺ T cells. In another embodiment, the term refers to an epitope recognized by over 4% of the antigen-specific CD8⁺ T cells. In another embodiment, the term refers to an epitope recognized by fewer than 3% of the antigen-specific CD8⁺ T cells. In another embodiment, the term refers to an epitope recognized by fewer than 2% of the antigen-specific CD8⁺ T cells. In another embodiment, the term refers to an epitope recognized by fewer than 1% of the antigen-specific CD8⁺ T cells. In another embodiment, the term refers to an epitope recognized by fewer than 0.5% of the antigen-specific CD8⁺ T cells.

Each type of the dominant epitope and subdominant epitope represents a separate embodiment of the present invention.

The antigen in methods and compositions of the present invention is, in one embodiment, expressed at a detectable level on a non-tumor cell of the subject. In another embodiment, the antigen is expressed at a detectable level on at least a certain percentage (e.g. 0.01%, 0.03%, 0.1%, 0.3%, 1%, 2%, 3%, or 5%) of non-tumor cells of the subject. In one embodiment, “non-tumor cell” refers to a cell outside the body of the tumor. In another embodiment, “non-tumor cell” refers to a non-malignant cell. In another embodiment, “non-tumor cell” refers to a non-transformed cell. In another embodiment, the non-tumor cell is a somatic cell. In another embodiment, the non-tumor cell is a germ cell. Each possibility represents a separate embodiment of the present invention.

“Detectable level” refers, in one embodiment, to a level that is detectable when using a standard assay. In one embodiment, the assay is an immunological assay. In one embodiment, the assay is enzyme-linked immunoassay (ELISA). In another embodiment, the assay is Western blot. In another embodiment, the assay is FACS. It is to be understood by a skilled artisan that any other assay available in the art can be used in the methods provided herein. In another embodiment, a detectable level is determined relative to the background level of a particular assay. Methods for performing each of these techniques are well known to those skilled in the art, and each technique represents a separate embodiment of the present invention.

In one embodiment, vaccination with recombinant antigen-expressing LM induces epitope spreading. In another embodiment, vaccination with LLO-antigen fusions, even outside the context of Her2, induces epitope spreading as well. Each possibility represents a separate embodiment of the present invention.

In another embodiment, the present invention provides a method of impeding a growth of an Her-2-expressing tumor in a subject, comprising administering to the subject a recombinant polypeptide comprising an N-terminal fragment of a LLO protein fused to a Her-2 chimeric antigen, wherein the antigen has one or more subdominant CD8⁺ T cell epitopes, wherein the subject mounts an immune response against the antigen-expressing tumor, thereby impeding a growth of an Her-2-expressing tumor in a subject. In another embodiment, the antigen does not contain any of the dominant CD8⁺ T cell epitopes. In another embodiment, provided herein is a method of impeding a growth on a Her-2-expressing tumor in a subject, comprising administering to the subject a recombinant form of Listeria comprising a recombinant nucleotide encoding the recombinant polypeptide provided herein.

In another embodiment, the present invention provides a method for inducing formation of cytotoxic T cells in a host having cancer, comprising administering to the host a composition of the present invention, thereby inducing formation of cytotoxic T cells in a host having cancer.

In another embodiment, the present invention provides a method of reducing an incidence of cancer, comprising administering a composition of the present invention. In another embodiment, the present invention provides a method of ameliorating cancer, comprising administering a composition of the present invention. Each possibility represents a separate embodiment of the present invention.

In one embodiment, the composition is administered to the cells of the subject ex vivo; in another embodiment, the composition is administered to the cells of a donor ex vivo; in another embodiment, the composition is administered to the cells of a donor in vivo, then is transferred to the subject. Each possibility represents a separate embodiment of the present invention.

In one embodiment, the cancer treated by a method of the present invention is breast cancer. In another embodiment, the cancer is a Her2 containing cancer. In another embodiment, the cancer is a melanoma. In another embodiment, the cancer is pancreatic cancer. In another embodiment, the cancer is ovarian cancer. In another embodiment, the cancer is gastric cancer. In another embodiment, the cancer is a carcinomatous lesion of the pancreas. In another embodiment, the cancer is pulmonary adenocarcinoma. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non small-cell lung carcinoma. In another embodiment, the cancer is a CNS carcinoma. In another embodiment, the cancer is an endometrial carcinoma. In another embodiment, the cancer is a bladder cancer. In another embodiment, the cancer is mesothelioma. In another embodiment, the cancer is malignant mesothelioma (MM). In another embodiment, the cancer is a head and neck cancer. In another embodiment, the cancer is a prostate carcinoma. In another embodiment, the cancer is a Her-2/neu expressing osteosarcoma. In another embodiment, the osteosarcoma is localized osteosarcoma. In another embodiment, the osteosarcoma is metastatic osteosarcoma. In another embodiment, the osteosarcoma is high grade osteosarcoma. Each possibility represents a separate embodiment of the present invention.

In another embodiment of the methods of the present invention, the subject mounts an immune response against the antigen-expressing tumor or target antigen, thereby mediating the anti-tumor effects.

In another embodiment, the present invention provides an immunogenic composition for treating cancer, the composition comprising a fusion of a truncated LLO to a Her-2 chimeric protein. In another embodiment, the immunogenic composition further comprises a Listeria strain expressing the fusion. Each possibility represents a separate embodiment of the present invention. In another embodiment, the present invention provides an immunogenic composition for treating cancer, the composition comprising a Listeria strain expressing a Her-2 chimeric protein.

In one embodiment, a treatment protocol of the present invention is therapeutic. In another embodiment, the protocol is prophylactic. In another embodiment, the vaccines of the present invention are used to protect people at risk for cancer such as breast cancer or other types of Her2-containing tumors because of familial genetics or other circumstances that predispose them to these types of ailments as will be understood by a skilled artisan. In another embodiment, the vaccines are used as a cancer immunotherapy after debulking of tumor growth by surgery, conventional chemotherapy or radiation treatment. Following such treatments, the vaccines of the present invention are administered so that the CTL response to the tumor antigen of the vaccine destroys remaining metastases and prolongs remission from the cancer. In another embodiment, vaccines are used as a cancer immunotherapy in combination with surgery, conventional chemotherapy or radiation treatment. In another embodiment, such combination treatment is used in subjects that cannot undergo amputation. In another embodiment, such combination treatment is used in subjects with primary osteosarcoma that cannot undergo amputation. In another embodiment, vaccines of the present invention are used to effect the growth of previously established tumors and to kill existing tumor cells. Each possibility represents a separate embodiment of the present invention.

In another embodiment, the vaccines and immunogenic compositions utilized in any of the methods described above have any of the characteristics of vaccines and immunogenic compositions of the present invention. Each characteristic represents a separate embodiment of the present invention.

Various embodiments of dosage ranges are contemplated by this invention. In one embodiment, in the case of vaccine vectors, the dosage is in the range of 0.4 LD₅₀/dose. In another embodiment, the dosage is from about 0.4-4.9 LD₅₀/dose. In another embodiment the dosage is from about 0.5-0.59 LD₅₀/dose. In another embodiment the dosage is from about 0.6-0.69 LD₅₀/dose. In another embodiment the dosage is from about 0.7-0.79 LD₅₀/dose. In another embodiment the dosage is about 0.8 LD₅₀/dose. In another embodiment, the dosage is 0.4 LD₅₀/dose to 0.8 of the LD₅₀/dose.

In another embodiment, the dosage is 10⁷ bacteria/dose. In another embodiment, the dosage is 1.5×10⁷ bacteria/dose. In another embodiment, the dosage is 2×10⁷ bacteria/dose.

In another embodiment, the dosage is 3×10⁷ bacteria/dose. In another embodiment, the dosage is 4×10⁷ bacteria/dose. In another embodiment, the dosage is 6×10⁷ bacteria/dose. In another embodiment, the dosage is 8×10⁷ bacteria/dose. In another embodiment, the dosage is 1×10⁸ bacteria/dose. In another embodiment, the dosage is 1.5×10⁸ bacteria/dose. In another embodiment, the dosage is 2×10⁸ bacteria/dose. In another embodiment, the dosage is 3×10⁸ bacteria/dose. In another embodiment, the dosage is 4×10⁸ bacteria/dose. In another embodiment, the dosage is 6×10⁸ bacteria/dose. In another embodiment, the dosage is 8×10⁸ bacteria/dose. In another embodiment, the dosage is 1×10⁹ bacteria/dose. In another embodiment, the dosage is 1.5×10⁹ bacteria/dose. In another embodiment, the dosage is 2×10⁹ bacteria/dose. In another embodiment, the dosage is 3×10⁹ bacteria/dose. In another embodiment, the dosage is 5×10⁹ bacteria/dose. In another embodiment, the dosage is 6×10⁹ bacteria/dose. In another embodiment, the dosage is 8×10⁹ bacteria/dose. In another embodiment, the dosage is 1×10¹⁰ bacteria/dose. In another embodiment, the dosage is 1.5×10¹⁰ bacteria/dose. In another embodiment, the dosage is 2×10¹⁰ bacteria/dose. In another embodiment, the dosage is 3×10¹⁰ bacteria/dose. In another embodiment, the dosage is 5×10¹⁰ bacteria/dose. In another embodiment, the dosage is 6×10¹⁰ bacteria/dose. In another embodiment, the dosage is 8×10¹⁰ bacteria/dose. In another embodiment, the dosage is 8×10⁹ bacteria/dose. In another embodiment, the dosage is 1×10¹¹ bacteria/dose. In another embodiment, the dosage is 1.5×10¹¹ bacteria/dose. In another embodiment, the dosage is 2×10¹¹ bacteria/dose. In another embodiment, the dosage is 3×10¹¹ bacteria/dose.

In another embodiment, the dosage is 5×10¹¹ bacteria/dose. In another embodiment, the dosage is 6×10¹¹ bacteria/dose. In another embodiment, the dosage is 8×10¹¹ bacteria/dose. Each possibility represents a separate embodiment of the present invention.

In one embodiment, a vaccine or immunogenic composition of the present invention is administered alone to a subject. In another embodiment, the vaccine or immunogenic composition is administered together with another cancer therapy. Each possibility represents a separate embodiment of the present invention.

The recombinant Listeria of methods and compositions of the present invention is, in one embodiment, stably transformed with a construct encoding a Her-2 chimeric antigen or an LLO-Her-2 chimeric antigen fusion. In one embodiment, the construct contains a polylinker to facilitate further subcloning. Several techniques for producing recombinant Listeria are known.

In one embodiment, the construct or nucleic acid molecule is integrated into the Listerial chromosome using homologous recombination. Techniques for homologous recombination are well known in the art, and are described, for example, in Baloglu S, Boyle S M, et al (Immune responses of mice to vaccinia virus recombinants expressing either Listeria monocytogenes partial listeriolysin or Brucella abortus ribosomal L7/L12 protein. Vet Microbiol 2005, 109(1-2): 11-7); and Jiang L L, Song H H, et al., (Characterization of a mutant Listeria monocytogenes strain expressing green fluorescent protein. Acta Biochim Biophys Sin(Shanghai) 2005, 37(1): 19-24). In another embodiment, homologous recombination is performed as described in U.S. Pat. No. 6,855,320. In this case, a recombinant LM strain that expresses E7 was made by chromosomal integration of the E7 gene under the control of the hly promoter and with the inclusion of the hly signal sequence to ensure secretion of the gene product, yielding the recombinant referred to as Lm-AZ/E7. In another embodiment, a temperature sensitive plasmid is used to select the recombinants. Each technique represents a separate embodiment of the present invention.

In another embodiment, the construct or nucleic acid molecule is integrated into the Listerial chromosome using transposon insertion. Techniques for transposon insertion are well known in the art, and are described, inter alia, by Sun et al. (Infection and Immunity 1990, 58: 3770-3778) in the construction of DP-L967. Transposon mutagenesis has the advantage, in another embodiment, that a stable genomic insertion mutant can be formed but the disadvantage that the position in the genome where the foreign gene has been inserted is unknown.

In another embodiment, the construct or nucleic acid molecule is integrated into the Listerial chromosome using phage integration sites (Lauer P, Chow M Y et al, Construction, characterization, and use of two Listeria monocytogenes site-specific phage integration vectors. J Bacteriol 2002; 184(15): 4177-86). In certain embodiments of this method, an integrase gene and attachment site of a bacteriophage (e.g. U153 or PSA listeriophage) is used to insert the heterologous gene into the corresponding attachment site, which may be any appropriate site in the genome (e.g. comK or the 3′ end of the arg tRNA gene). In another embodiment, endogenous prophages are cured from the attachment site utilized prior to integration of the construct or heterologous gene. In another embodiment, this method results in single-copy integrants. Each possibility represents a separate embodiment of the present invention.

In another embodiment, one of various promoters is used to express the antigen or fusion protein containing same. In one embodiment, an LM promoter is used, e.g. promoters for the genes hly, actA, pica, plcB and mpl, which encode the Listerial proteins hemolysin, actA, phosphotidylinositol-specific phospholipase, phospholipase C, and metalloprotease, respectively. Each possibility represents a separate embodiment of the present invention.

In another embodiment, methods and compositions of the present invention utilize a homologue of a Her-2 chimeric protein or LLO sequence of the present invention. In another embodiment, the methods and compositions of the present invention utilize a Her-2 chimeric protein from a non-human mammal. The terms “homology,” “homologous,” etc, when in reference to any protein or peptide, refer in one embodiment, to a percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Methods and computer programs for the alignment are well known in the art.

In another embodiment, the term “homology,” when in reference to any nucleic acid sequence similarly indicates a percentage of nucleotides in a candidate sequence that are identical with the nucleotides of a corresponding native nucleic acid sequence.

In another embodiment, the present invention provides an isolated nucleic acid encoding a signal peptide or a recombinant polypeptide or fusion protein of the present invention. In one embodiment, the isolated nucleic acid comprises a sequence sharing at least 65% homology with a nucleic acid encoding the signal peptide or the recombinant polypeptide or the fusion protein of the present invention. In another embodiment, the isolated nucleic acid comprises a sequence sharing at least 75% homology with a nucleic acid encoding the signal peptide or the recombinant polypeptide or the fusion protein of the present invention. In another embodiment, the isolated nucleic acid comprises a sequence sharing at least 85% homology with a nucleic acid encoding the signal peptide or the recombinant polypeptide or the fusion protein of the present invention. In another embodiment, the isolated nucleic acid comprises a sequence sharing at least 90% homology with a nucleic acid encoding the signal peptide or the recombinant polypeptide or the fusion protein of the present invention. In another embodiment, the isolated nucleic acid comprises a sequence sharing at least 95% homology with a nucleic acid encoding the signal peptide or the recombinant polypeptide or the fusion protein of the present invention. In another embodiment, the isolated nucleic acid comprises a sequence sharing at least 97% homology with a nucleic acid encoding the signal peptide or the recombinant polypeptide or the fusion protein of the present invention. In another embodiment, the isolated nucleic acid comprises a sequence sharing at least 99% homology with a nucleic acid encoding the signal peptide or the recombinant polypeptide or the fusion protein of the present invention.

Homology is, in one embodiment, determined by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a sequence selected from a sequence (nucleic acid or amino acid sequence) provided herein of greater than 65%. In another embodiment, “homology” refers to identity to a sequence selected from a sequence provided herein of greater than 70%. In another embodiment, the identity is greater than 75%. In another embodiment, the identity is greater than 78%. In another embodiment, the identity is greater than 80%. In another embodiment, the identity is greater than 82%. In another embodiment, the identity is greater than 83%. In another embodiment, the identity is greater than 85%. In another embodiment, the identity is greater than 87%. In another embodiment, the identity is greater than 88%. In another embodiment, the identity is greater than 90%. In another embodiment, the identity is greater than 92%. In another embodiment, the identity is greater than 93%. In another embodiment, the identity is greater than 95%. In another embodiment, the identity is greater than 96%. In another embodiment, the identity is greater than 97%. In another embodiment, the identity is greater than 98%. In another embodiment, the identity is greater than 99%. In another embodiment, the identity is 100%. Each possibility represents a separate embodiment of the present invention.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and Ausubel et al., 1989, Current Protocols in Molecular Biology, Green Publishing Associates and Wiley Interscience, N.Y.). For example methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5×Denhardt's solution, 10% dextran sulfate, and 20 ng/ml denatured, sheared salmon sperm DNA.

In one embodiment of the present invention, “nucleic acids” refers to a string of at least two base-sugar-phosphate combinations. The term includes, in one embodiment, DNA and RNA. “Nucleotides” refers, in one embodiment, to the monomeric units of nucleic acid polymers. RNA may be, in one embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). DNA may be in form of plasmid DNA, viral DNA, linear DNA, or chromosomal DNA or derivatives of these groups. In addition, these forms of DNA and RNA may be single, double, triple, or quadruple stranded. The term also includes, in another embodiment, artificial nucleic acids that may contain other types of backbones but the same bases. In one embodiment, the artificial nucleic acid is a PNA (peptide nucleic acid). PNA contain peptide backbones and nucleotide bases and are able to bind, in one embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothiorate nucleic acids and PNA are known to those skilled in the art, and are described in, for example, Neilsen P E, Curr Opin Struct Biol 9:353-57; and Raz N K et al Biochem Biophys Res Commun 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed. Each nucleic acid derivative represents a separate embodiment of the present invention.

Protein and/or peptide homology for any amino acid sequence listed herein is determined, in one embodiment, by methods well described in the art, including immunoblot analysis, or via computer algorithm analysis of amino acid sequences, utilizing any of a number of software packages available, via established methods. Some of these packages may include the FASTA, BLAST, MPsrch or Scanps packages, and may employ the use of the Smith and Waterman algorithms, and/or global/local or BLOCKS alignments for analysis, for example. Each method of determining homology represents a separate embodiment of the present invention.

In another embodiment, the present invention provides a kit comprising a reagent utilized in performing a method of the present invention. In another embodiment, the present invention provides a kit comprising a composition, tool, or instrument of the present invention.

The terms “contacting” or “administering,” in one embodiment, refer to directly contacting the cancer cell or tumor with a composition of the present invention. In another embodiment, the terms refer to indirectly contacting the cancer cell or tumor with a composition of the present invention. In another embodiment, methods of the present invention include methods in which the subject is contacted with a composition of the present invention after which the composition is brought in contact with the cancer cell or tumor by diffusion or any other active transport or passive transport process known in the art by which compounds circulate within the body. Each possibility represents a separate embodiment of the present invention.

In another embodiment, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals or organisms. This can be readily carried out by using hybridization probes to identify the same genetic locus in a variety of individuals or organisms. Any and all such nucleotide variations and resulting amino acid polymorphisms or variations that are the result of natural allelic variation and that do not alter the functional activity are intended to be within the scope of the invention.

### Pharmaceutical Compositions

The pharmaceutical compositions containing vaccines and compositions of the present invention are, in another embodiment, administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally.

In another embodiment of the methods and compositions provided herein, the vaccines or compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound and the inert carrier or diluent, a hard gelating capsule.

In another embodiment, the vaccines or compositions are administered by intravenous, intra-arterial, or intra-muscular injection of a liquid preparation. Suitable liquid formulations include solutions, suspensions, dispersions, emulsions, oils and the like. In one embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In one embodiment, the term “treating” refers to curing a disease. In another embodiment, “treating” refers to preventing a disease. In another embodiment, “treating” refers to reducing the incidence of a disease. In another embodiment, “treating” refers to ameliorating symptoms of a disease. In another embodiment, “treating” refers to increasing performance free survival or overall survival of a patient. In another embodiment, “treating” refers to stabilizing the progression of a disease. In another embodiment, “treating” refers to inducing remission. In another embodiment, “treating” refers to slowing the progression of a disease. The terms “reducing”, “suppressing” and “inhibiting” refer in another embodiment to lessening or decreasing. Each possibility represents a separate embodiment of the present invention.

The term “about” as used herein means in quantitative terms plus or minus 5%, or in another embodiment plus or minus 10%, or in another embodiment plus or minus 15%, or in another embodiment plus or minus 20%. It is to be understood by the skilled artisan that the term “subject” can encompass a mammal including an adult human or a human child, teenager or adolescent in need of therapy for, or susceptible to, a condition or its sequelae, and also may include non-human mammals such as dogs, cats, pigs, cows, sheep, goats, horses, rats, and mice. It will also be appreciated that the term may encompass livestock. The term “subject” does not exclude an individual that is normal in all respects.

In one embodiment, the term “subject” also encompasses dogs that cannot undergo amputation.

It will be appreciated by the skilled artisan that the term “mammal” for purposes of treatment refers to any animal classified as a mammal, including, but not limited to, humans, domestic and farm animals, and zoo, sports, or pet animals, such as canines, including dogs, and horses, cats, cattle, pigs, sheep, etc.

A “therapeutically effective amount”, in reference to the treatment of tumor, refers to an amount capable of invoking one or more of the following effects: (1) inhibition, to some extent, of tumor growth, including, slowing down and complete growth arrest; (2) reduction in the number of tumor cells; (3) reduction in tumor size; (4) inhibition (i.e., reduction, slowing down or complete stopping) of tumor cell infiltration into peripheral organs; (5) inhibition (i.e., reduction, slowing down or complete stopping) of metastasis; (6) enhancement of anti-tumor immune response, which may, but does not have to, result in the regression or rejection of the tumor; and/or (7) relief, to some extent, of one or more symptoms associated with the disorder. A “therapeutically effective amount” of a vaccine provided herein for purposes of treatment of tumor may be determined empirically and in a routine manner.

The following examples are presented in order to more fully illustrate the preferred embodiments of the invention. They should in no way be construed, however, as limiting the broad scope of the invention.

### EXAMPLES

### Materials and Methods

Oligonucleotides were synthesized by Invitrogen (Carlsbad, Calif.) and DNA sequencing was done by Genewiz Inc, South Plainfield, N.J. Flow cytometry reagents were purchased from Becton Dickinson Biosciences (BD, San Diego, Calif.). Cell culture media, supplements and all other reagents, unless indicated, were from Sigma (St. Louise, Mo.). Her2/neu HLA-A2 peptides were synthesized by EZbiolabs (Westfield, Ind.). Complete RPMI 1640 (C—RPMI) medium contained 2 mM glutamine, 0.1 mM non-essential amino acids, and 1 mM sodium pyruvate, 10% fetal bovine serum, penicillin/streptomycin, Hepes (25 mM). The polyclonal anti-LLO antibody was described previously and anti-Her2/neu antibody was purchased from Sigma.

### Mice and Cell Lines

All animal experiments were performed according to approved protocols by IACUC at the University of Pennsylvania or Rutgers University. FVB/N mice were purchased from Jackson laboratories (Bar Harbor, Me.). The FVB/N Her2/neu transgenic mice, which overexpress the rat Her2/neu onco-protein were housed and bred at the animal core facility at the University of Pennsylvania. The NT-2 tumor cell line expresses high levels of rat Her2/neu protein, was derived from a spontaneous mammary tumor in these mice and grown as described previously. DHFR-G8 (3T3/neu) cells were obtained from ATCC and were grown according to the ATCC recommendations. The EMT6-Luc cell line was a generous gift from Dr. John Ohlfest (University of Minnesota, Minn.) and was grown in complete C—RPMI medium. Bioluminescent work was conducted under guidance by the Small Animal Imaging Facility (SAIF) at the University of Pennsylvania (Philadelphia, Pa.).

### Listeria Constructs and Antigen Expression

Her2/neu-pGEM7Z was kindly provided by Dr. Mark Greene at the University of Pennsylvania and contained the full-length human Her2/neu (hHer2) gene cloned into the pGEM7Z plasmid (Promega, Madison Wis.). This plasmid was used as a template to amplify three segments of hHer-2/neu, namely, EC1, EC2, and IC1, by PCR using pfx DNA polymerase (Invitrogen) and the oligos indicated in Table 1.

TABLE 1
Primers for cloning of Human her-2-Chimera

| | | | Amino acid |
|---|---|---|---|
| | | Base pair | region or |
| | DNA sequence | region | junctions |
| Her-2- | TGATCTCGAGACCCACCTGGACATGCTC (SEQ ID NO: 57) | 120-510 | 40-170 |
| Chimera (F) | | | |
| HerEC1- | CTACCAGGACACGATTTTGTGGAAG-AATATCCA | 510/1077 | 170/359 |
| EC2F | GGAGTTTGCTGGCTGC (SEQ ID NO: 58) | | |
| (Junction) | | | |
| HerEC1- | GCAGCCAGCAAACTCCTGGATATT-CTTCCACAA | | |
| EC2R | AATCGTGTCCTGGTAG (SEQ ID NO: 59) | | |
| (Junction) | | | |
| HerEC2- | CTGCCACCAGCTGTGCGCCCGAGGG- | 1554/2034 | 518/679 |
| ICIF | CAGCAGAAGATCCGGAAGTACACGA (SEQ ID NO: 60) | | |
| (Junction) | | | |
| HerEC2- | TCGTGTACTTCCGGATCTTCTGCTG | | |
| ICIR | CCCTCGGGC GCACAGCTGGTGGCAG (SEQ ID NO: 61) | | |
| (Junction) | | | |
| Her-2- | GTGGCCCGGGTCTAGATTAGTCTAAGAGGCAGCCATAGG | 2034-2424 | 679-808 |
| Chimera (R) | (SEQ ID NO: 62) | | |

The Her-2/neu chimera construct was generated by direct fusion by the SOEing PCR method and each separate hHer-2/neu segment as templates. Primers are shown in Table 2.

Sequence of primers for amplification of different segments human Her2 regions

| | | | Amino acid |
|---|---|---|---|
| | DNA sequence | Base pair region | region |
| Her-2-EC1(F) | CCGCCTCGAGGCCGCGAGCACCCAAGTG | 58-979 | 20-326 |
| | (SEQ ID NO: 63) | | |
| Her-2-EC1(R) | CGCGACTAGTTTAATCCTCTGCTGTCACCTC | | |
| | (SEQ ID NO: 64) | | |
| Her-2-EC2(F) | CCGCCTCGAGTACCTTTCTACGGACGTG (SEQ | 907-1504 | 303-501 |
| | ID NO: 65) | | |
| Her-2-EC2(R) | CGCGACTAGTTTACTCTGGCCGGTTGGCAG | | |
| | (SEQ ID NO: 66) | | |
| Her-2-Her-2- | CCGCCTCGAGCAGCAGAAGATCCGGAAGTAC | 2034-3243 | 679-1081 |
| IC1(F) | (SEQ ID NO: 67) | | |
| Her-2-IC1(R) | CGCGACTAGTTTAAGCCCCTTCGGAGGGTG | | |
| | (SEQ ID NO: 68) | | |

ChHer2 gene was excised from pAdv138 using XhoI and SpeI restriction enzymes, and cloned in frame with a truncated, non-hemolytic fragment of LLO in the Lmdd shuttle vector, pAdv134. The sequences of the insert, LLO and hly promoter were confirmed by DNA sequencing analysis. This plasmid was electroporated into electro-competent actA, dal, dat mutant Listeria monocytogenes strain, LmddA and positive clones were selected on Brain Heart infusion (BHI) agar plates containing streptomycin (250 μg/ml). In some experiments similar Listeria strains expressing hHer2/neu (Lm-hHer2) fragments were used for comparative purposes. These have been previously described. In all studies, an irrelevant Listeria construct (Lm-control) was included to account for the antigen independent effects of Listeria on the immune system. Lm-controls were based on the same Listeria platform as ADXS31-164, but expressed a different antigen such as HPV16-E7 or NY-ESO-1. Expression and secretion of fusion proteins from Listeria were tested. Each construct was passaged twice in vivo.

### Cytotoxicity Assay

Groups of 3-5 FVB/N mice were immunized three times with one week intervals with 1×10⁸ colony forming units (CFU) of Lm-LLO-ChHer2, ADXS31-164, Lm-hHer2 ICI or Lm-control (expressing an irrelevant antigen) or were left naïve. NT-2 cells were grown in vitro, detached by trypsin and treated with mitomycin C (250 μg/ml in serum free C—RPMI medium) at 37° C. for 45 minutes. After 5 washes, they were co-incubated with splenocytes harvested from immunized or naïve animals at a ratio of 1:5 (Stimulator:Responder) for 5 days at 37° C. and 5% CO₂. A standard cytotoxicity assay was performed using europium labeled 3T3/neu (DHFR-G8) cells as targets according to the method previously described. Released europium from killed target cells was measured after 4 hour incubation using a spectrophotometer (Perkin Elmer, Victor²) at 590 nm Percent specific lysis was defined as (lysis in experimental group-spontaneous lysis)/(Maximum lysis-spontaneous lysis).

Interferon-γ Secretion by Splenocytes from Immunized Mice

Groups of 3-5 FVB/N or HLA-A2 transgenic mice were immunized three times with one week intervals with 1×10⁸ CFU of ADXS31-164, a negative Listeria control (expressing an irrelevant antigen) or were left naïve. Splenocytes from FVB/N mice were isolated one week after the last immunization and co-cultured in 24 well plates at 5×10⁶ cells/well in the presence of mitomycin C treated NT-2 cells in C—RPMI medium. Splenocytes from the HLA-A2 transgenic mice were incubated in the presence of 1 μM of HLA-A2 specific peptides or 1 μg/ml of a recombinant His-tagged ChHer2 protein, produced in E. coli and purified by a nickel based affinity chromatography system. Samples from supernatants were obtained 24 or 72 hours later and tested for the presence of interferon-γ (IFN-γ) using mouse IFN-γ Enzyme-linked immunosorbent assay (ELISA) kit according to manufacturer's recommendations.

Tumor Studies in her2 Transgenic Animals

Six weeks old FVB/N rat Her2/neu transgenic mice (9-14/group) were immunized 6 times with 5×10⁸ CFU of Lm-LLO-ChHer2, ADXS31-164 or Lm-control. They were observed twice a week for the emergence of spontaneous mammary tumors, which were measured using an electronic caliper, for up to 52 weeks. Escaped tumors were excised when they reached a size 1 cm² in average diameter and preserved in RNA later at −20° C. In order to determine the effect of mutations in the Her2/neu protein on the escape of these tumors, genomic DNA was extracted using a genomic DNA isolation kit, and sequenced.

### Effect of ADXS31-164 on Regulatory T Cells in Spleens and Tumors

Mice were implanted subcutaneously (s.c.) with 1×10⁶ NT-2 cells. On days 7, 14 and 21, they were immunized with 1×10⁸ CFUs of ADXS31-164, LmddA-control or left naïve. Tumors and spleens were extracted on day 28 and tested for the presence of CD3⁺/CD4⁺/FoxP3⁺ Tregs by FACS analysis. Briefly, splenocytes were isolated by homogenizing the spleens between two glass slides in C—RPMI medium. Tumors were minced using a sterile razor blade and digested with a buffer containing DNase (12 U/ml), and collagenase (2 mg/ml) in PBS. After 60 min incubation at RT with agitation, cells were separated by vigorous pipetting. Red blood cells were lysed by RBC lysis buffer followed by several washes with complete RPMI-1640 medium containing 10% FBS. After filtration through a nylon mesh, tumor cells and splenocytes were resuspended in FACS buffer (2% FBS/PBS) and stained with anti-CD3-PerCP-Cy5.5, CD4-FITC, CD25-APC antibodies followed by permeabilization and staining with anti-Foxp3-PE. Flow cytometry analysis was performed using 4-color FACS calibur (BD) and data were analyzed using cell quest software (BD).

### Statistical Analysis

The log-rank Chi-Squared test was used for survival data and student's t-test for the CTL and ELISA assays, which were done in triplicates. A p-value of less than 0.05 (marked as *) was considered statistically significant in these analyzes. All statistical analysis was done with either Prism software, V.4.0a (2006) or SPSS software, V.15.0 (2006). For all FVB/N rat Her2/neu transgenic studies we used 8-14 mice per group, for all wild-type FVB/N studies we used at least 8 mice per group unless otherwise stated. All studies were repeated at least once except for the long term tumor study in Her2/neu transgenic mouse model.

### Example 1

### Generation of L. Monocytogenes Strains that Secrete LLO Fragments Fused to Her-2 Fragments: Construction of ADXS31-164

Construction of the chimeric Her2/neu gene (ChHer2) was described previously. Briefly, ChHer2 gene was generated by direct fusion of two extracellular (aa 40-170 and aa 359-433) and one intracellular fragment (aa 678-808) of the Her2/neu protein by SOEing PCR method. The chimeric protein harbors most of the known human MHC class I epitopes of the protein. ChHer2 gene was excised from the plasmid, pAdv138 (which was used to construct Lm-LLO-ChHer2) and cloned into LmddA shuttle plasmid, resulting in the plasmid pAdv164 (FIG. 1A). There are two major differences between these two plasmid backbones. 1) Whereas pAdv138 uses the chloramphenicol resistance marker (cat) for in vitro selection of recombinant bacteria, pAdv164 harbors the D-alanine racemase gene (dal) from bacillus subtilis, which uses a metabolic complementation pathway for in vitro selection and in vivo plasmid retention in LmddA strain which lacks the dal-dat genes. This vaccine platform was designed and developed to address FDA concerns about the antibiotic resistance of the engineered Listeria vaccine strains. 2) Unlike pAdv138, pAdv164 does not harbor a copy of the prfA gene in the plasmid (see sequence below and FIG. 1A), as this is not necessary for in vivo complementation of the Lmdd strain. The LmddA vaccine strain also lacks the actA gene (responsible for the intracellular movement and cell-to-cell spread of Listeria) so the recombinant vaccine strains derived from this backbone are 100 times less virulent than those derived from the Lmdd, its parent strain. LmddA-based vaccines are also cleared much faster (in less than 48 hours) than the Lmdd-based vaccines from the spleens of the immunized mice. The expression and secretion of the fusion protein tLLO-ChHer2 from this strain was comparable to that of the Lm-LLO-ChHer2 in TCA precipitated cell culture supernatants after 8 hours of in vitro growth (FIG. 1B) as a band of ˜104 KD was detected by an anti-LLO antibody using Western Blot analysis. The Listeria backbone strain expressing only tLLO was used as negative control.

pAdv164 sequence (7075 base pairs) (see FIG. 1):

(SED ID NO: 53)

cggagtgtatactggcttactatgttggcactgatgagggtgtcagtgaagtgcttcatgtggcaggagaaaaaaggctgcaccggtgc
gtcagcagaatatgtgatacaggatatattccgatcctcgctcactgactcgctacgctcggtcgttcgactgcggcgagcggaaatgg
cttacgaacggggcggagatttcctggaagatgccaggaagatacttaacagggaagtgagagggccgcggcaaagccgtttttcca
taggctccgcccccctgacaagcatcacgaaatctgacgctcaaatcagtggtggcgaaacccgacaggactataaagataccaggc
gtttccccctggcggctccctcgtgcgctctcctgttcctgcctttcggtttaccggtgtcattccgctgttatggccgcgtttgtctcattcca
cgcctgacactcagttccgggtaggcagttcgctccaagctggactgtatgcacgaaccccccgttcagtccgaccgctgcgccttatc
cggtaactatcgtcttgagtccaacccggaaagacatgcaaaagcaccactggcagcagccactggtaattgatttagaggagttagtc
ttgaagtcatgcgccggttaaggctaaactgaaaggacaagttttggtgactgcgctcctccaagccagttacctcggttcaaagagttg
gtagctcagagaaccttcgaaaaaccgccctgcaaggcggttttttcgttttcagagcaagagattacgcgcagaccaaaacgatctca
agaagatcatcttattaatcagataaaatatttctagccctcctttgattagtatattcctatcttaaagttacttttatgtggaggcattaacattt
gttaatgacgtcaaaaggatagcaagactagaataaagctataaagcaagcatataatattgcgtttcatctttagaagcgaatttcgcca
atattataattatcaaaagagaggggtggcaaacggtatttggcattattaggttaaaaaatgtagaaggagagtgaaacccatgaaaaa
aataatgctagtttttattacacttatattagttagtctaccaattgcgcaacaaactgaagcaaaggatgcatctgcattcaataaagaaaat
tcaatttcatccatggcaccaccagcatctccgcctgcaagtcctaagacgccaatcgaaaagaaacacgcggatgaaatcgataagta
tatacaaggattggattacaataaaaacaatgtattagtataccacggagatgcagtgacaaatgtgccgccaagaaaaggttacaaag
atggaaatgaatatattgttgtggagaaaaagaagaaatccatcaatcaaaataatgcagacattcaagttgtgaatgcaatttcgagcct
aacctatccaggtgctctcgtaaaagcgaattcggaattagtagaaaatcaaccagatgttctccctgtaaaacgtgattcattaacactca
gcattgatttgccaggtatgactaatcaagacaataaaatagttgtaaaaaatgccactaaatcaaacgttaacaacgcagtaaatacatta
gtggaaagatggaatgaaaaatatgctcaagcttatccaaatgtaagtgcaaaaattgattatgatgacgaaatggcttacagtgaatcac
aattaattgcgaaatttggtacagcatttaaagctgtaaataatagcttgaatgtaaacttcggcgcaatcagtgaagggaaaatgcaaga
agaagtcattagttttaaacaaatttactataacgtgaatgttaatgaacctacaagaccttccagatttttcggcaaagctgttactaaagag
cagttgcaagcgcttggagtgaatgcagaaaatcctcctgcatatatctcaagtgtggcgtatggccgtcaagtttatttgaaattatcaac
taattcccatagtactaaagtaaaagctgcttttgatgctgccgtaagcggaaaatctgtctcaggtgatgtagaactaacaaatatcatca
aaaattcttccttcaaagccgtaatttacggaggaccgcaaaagatgaagttcaaatcatcgacggcaacctcggagacttacgcgatat
tttgaaaaaaggcgctacttttaatcgagaaacaccaggagttcccattgcttatacaacaaacttcctaaaagacaatgaattagctgttat
taaaaacaactcagaatatattgaaacaacttcaaaagcttatacagatggaaaaattaacatcgatcactctggaggatacgttgctcaat
tcaacatttcttgggatgaagtaaattatgatctcgagacccacctggacatgctccgccacctctaccagggctgccaggtggtgcagg
gaaacctggaactcacctacctgcccaccaatgccagcctgtccttcctgcaggatatccaggaggtgcagggctacgtgctcatcgct
cacaaccaagtgaggcaggtcccactgcagaggctgcggattgtgcgaggcacccagctctttgaggacaactatgccctggccgtg
ctagacaatggagacccgctgaacaataccacccctgtcacaggggcctccccaggaggcctgcgggagctgcagcttcgaagcct
cacagagatcttgaaaggaggggtcttgatccagcggaacccccagctctgctaccaggacacgattttgtggaagaatatccaggag
tttgctggctgcaagaagatctttgggagcctggcatttctgccggagagctttgatggggacccagcctccaacactgccccgctcca
gccagagcagctccaagtgtttgagactctggaagagatcacaggttacctatacatctcagcatggccggacagcctgcctgacctca
gcgtcttccagaacctgcaagtaatccggggacgaattctgcacaatggcgcctactcgctgaccctgcaagggctgggcatcagctg
gctggggctgcgctcactgagggaactgggcagtggactggccctcatccaccataacacccacctctgcttcgtgcacacggtgcc
ctgggaccagctctttcggaacccgcaccaagctctgctccacactgccaaccggccagaggacgagtgtgtgggcgagggcctgg
cctgccaccagctgtgcgcccgagggcagcagaagatccggaagtacacgatgcggagactgctgcaggaaacggagctggtgg
agccgctgacacctagcggagcgatgcccaaccaggcgcagatgcggatcctgaaagagacggagctgaggaaggtgaaggtgc
ttggatctggcgcttttggcacagtctacaagggcatctggatccctgatggggagaatgtgaaaattccagtggccatcaaagtgttga
gggaaaacacatcccccaaagccaacaaagaaatcttagacgaagcatacgtgatggctggtgtgggctccccatatgtctcccgcct
tctgggcatctgcctgacatccacggtgcagctggtgacacagcttatgccctatggctgcctcttagactaatctagacccgggccact
aactcaacgctagtagtggatttaatcccaaatgagccaacagaaccagaaccagaaacagaacaagtaacattggagttagaaatgg
aagaagaaaaaagcaatgatttcgtgtgaataatgcacgaaatcattgcttatttttttaaaaagcgatatactagatataacgaaacaacg
aactgaataaagaatacaaaaaaagagccacgaccagttaaagcctgagaaactttaactgcgagccttaattgattaccaccaatcaat
taaagaagtcgagacccaaaatttggtaaagtatttaattactttattaatcagatacttaaatatctgtaaacccattatatcgggtttttgag
gggatttcaagtctttaagaagataccaggcaatcaattaagaaaaacttagttgattgccttttttgttgtgattcaactttgatcgtagcttct
aactaattaattttcgtaagaaaggagaacagctgaatgaatatcccttttgttgtagaaactgtgcttcatgacggcttgttaaagtacaaat
ttaaaaatagtaaaattcgctcaatcactaccaagccaggtaaaagtaaaggggctatttttgcgtatcgctcaaaaaaaagcatgattgg
cggacgtggcgttgttctgacttccgaagaagcgattcacgaaaatcaagatacatttacgcattggacaccaaacgtttatcgttatggt
acgtatgcagacgaaaaccgttcatacactaaaggacattctgaaaacaatttaagacaaatcaataccttctttattgattttgatattcaca
cggaaaaagaaactatttcagcaagcgatattttaacaacagctattgatttaggatttttgcctacgttaattatcaaatctgataaaggttat
caagcatattttgttttagaaacgccagtctatgtgacttcaaaatcagaatttaaatctgtcaaagcagccaaaataatctcgcaaaatatc
cgagaatattttggaaagtctttgccagttgatctaacgtgcaatcattttgggattgctcgtataccaagaacggacaatgtagaattttttg
atcccaattaccgttattctttcaaagaatggcaagattggtctttcaaacaaacagataataagggctttactcgttcaagtctaacggtttt
aagcggtacagaaggcaaaaaacaagtagatgaaccctggtttaatctcttattgcacgaaacgaaattttcaggagaaaagggtttagt
agggcgcaatagcgttatgtttaccctctctttagcctactttagttcaggctattcaatcgaaacgtgcgaatataatatgtttgagtttaata
atcgattagatcaacccttagaagaaaaagaagtaatcaaaattgttagaagtgcctattcagaaaactatcaaggggctaatagggaat
acattaccattctttgcaaagcttgggtatcaagtgatttaaccagtaaagatttatttgtccgtcaagggtggtttaaattcaagaaaaaaag
aagcgaacgtcaacgtgttcatttgtcagaatggaaagaagatttaatggcttatattagcgaaaaaagcgatgtatacaagccttatttag
cgacgaccaaaaaagagattagagaagtgctaggcattcctgaacggacattagataaattgctgaaggtactgaaggcgaatcagga
aattttctttaagattaaaccaggaagaaatggtggcattcaacttgctagtgttaaatcattgttgctatcgatcattaaattaaaaaaagaa
gaacgagaaagctatataaaggcgctgacagcttcgtttaatttagaacgtacatttattcaagaaactctaaacaaattggcagaacgcc
ccaaaacggacccacaactcgatttgtttagctacgatacaggctgaaaataaaacccgcactatgccattacatttatatctatgatacgt
gtttgtttttctttgctggctagcttaattgcttatatttacctgcaataaaggatttcttacttccattatactcccattttccaaaaacatacggg
gaacacgggaacttattgtacaggccacctcatagttaatggtttcgagccttcctgcaatctcatccatggaaatatattcatccccctgc
cggcctattaatgtgacttttgtgcccggcggatattcctgatccagctccaccataaattggtccatgcaaattcggccggcaattttcag
gcgttttcccttcacaaggatgtcggtccctttcaattttcggagccagccgtccgcatagcctacaggcaccgtcccgatccatgtgtctt
tttccgctgtgtactcggctccgtagctgacgctctcgccttttctgatcagtttgacatgtgacagtgtcgaatgcagggtaaatgccgga
cgcagctgaaacggtatctcgtccgacatgtcagcagacgggcgaaggccatacatgccgatgccgaatctgactgcattaaaaaag
cctttttttcagccggagtccagcggcgctgttcgcgcagtggaccattagattctttaacggcagcggagcaatcagctctttaaagcgct
caaactgcattaagaaatagcctctttctttttcatccgctgtcgcaaaatgggtaaatacccctttgcactttaaacgagggttgcggtcaa
gaattgccatcacgttctgaacttcttcctctgtttttacaccaagtctgttcatccccgtatcgaccttcagatgaaaatgaagagaacctttt
ttcgtgtggcgggctgcctcctgaagccattcaacagaataacctgttaaggtcacgtcatactcagcagcgattgccacatactccggg
ggaaccgcgccaagcaccaatataggcgccttcaatccctttttgcgcagtgaaatcgcttcatccaaaatggccacggccaagcatga
agcacctgcgtcaagagcagcctttgctgtttctgcatcaccatgcccgtaggcgtttgctttcacaactgccatcaagtggacatgttca
ccgatatgttttttcatattgctgacattttcctttatcgcggacaagtcaatttccgcccacgtatctctgtaaaaaggttttgtgctcatggaaaactcc
tctcttttttcagaaaatcccagtacgtaattaagtatttgagaattaattttatattgattaatactaagtttacccagttttcacctaaaaaacaaatgat
gagataatagctccaaaggctaaagaggactataccaactatttgttaattaa

### Example 2

### ADXS31-164 is as Immunogenic as LM-LLO-ChHer2

Immunogenic properties of ADXS31-164 in generating anti-Her2/neu specific cytotoxic T cells were compared to those of the Lm-LLO-ChHer2 vaccine in a standard CTL assay. Both vaccines elicited strong but comparable cytotoxic T cell responses toward Her2/neu antigen expressed by 3T3/neu target cells. Accordingly, mice immunized with a Listeria expressing only an intracellular fragment of Her2-fused to LLO showed lower lytic activity than the chimeras which contain more MHC class I epitopes. No CTL activity was detected in naïve animals or mice injected with the irrelevant Listeria vaccine (FIG. 2A). ADXS31-164 was also able to stimulate the secretion of IFN-γ by the splenocytes from wild type FVB/N mice (FIG. 2B). This was detected in the culture supernatants of these cells that were co-cultured with mitomycin C treated NT-2 cells, which express high levels of Her2/neu antigen (FIG. 5C).

Proper processing and presentation of the human MHC class I epitopes after immunizations with ADXS31-164 was tested in HLA-A2 mice. Splenocytes from immunized HLA-A2 transgenics were co-incubated for 72 hours with peptides corresponding to mapped HLA-A2 restricted epitopes located at the extracellular (HLYQGCQVV SEQ ID NO: 11 or KIFGSLAFL SEQ ID NO: 12) or intracellular (RLLQETELV SEQ ID NO: 13) domains of the Her2/neu molecule (FIG. 2C). A recombinant ChHer2 protein was used as positive control and an irrelevant peptide or no peptide as negative controls. The data from this experiment show that ADXS31-164 is able to elicit anti-Her2/neu specific immune responses to human epitopes that are located at different domains of the targeted antigen.

### Example 3

### ADXS31-164 was More Efficacious Than LM-LLO-ChHER2 in Preventing the Onset of Spontaneous Mammary Tumors

Anti-tumor effects of ADXS31-164 were compared to those of Lm-LLO-ChHer2 in Her2/neu transgenic animals which develop slow growing, spontaneous mammary tumors at 20-25 weeks of age. All animals immunized with the irrelevant Listeria-control vaccine developed breast tumors within weeks 21-25 and were sacrificed before week 33. In contrast, Liseria-Her2/neu recombinant vaccines caused a significant delay in the formation of the mammary tumors. On week 45, more than 50% o ADXS31-164 vaccinated mice (5 out of 9) were still tumor free, as compared to 25% of mice immunized with Lm-LLO-ChHer2. At week 52, 2 out of 8 mice immunized with ADXS31-164 still remained tumor free, whereas all mice from other experimental groups had already succumbed to their disease (FIG. 3). These results indicate that despite being more attenuated, ADXS31-164 is more efficacious than Lm-LLO-ChHer2 in preventing the onset of spontaneous mammary tumors in Her2/neu transgenic animals.

### Example 4

### Mutations in Her2/Neu Gene Upon Immunization with ADXS31-164

Mutations in the MHC class I epitopes of Her2/neu have been considered responsible for tumor escape upon immunization with small fragment vaccines or trastuzumab (Herceptin), a monoclonal antibody that targets an epitope in the extracellular domain of Her2/neu. To assess this, genomic material was extracted from the escaped tumors in the transgenic animals and sequenced the corresponding fragments of the neu gene in tumors immunized with the chimeric or control vaccines. Mutations were not observed within the Her-2/neu gene of any vaccinated tumor samples suggesting alternative escape mechanisms (data not shown).

### Example 5

### Adxs31-164 Causes a Significant Decrease in Intra-Tumoral T Regulatory Cells

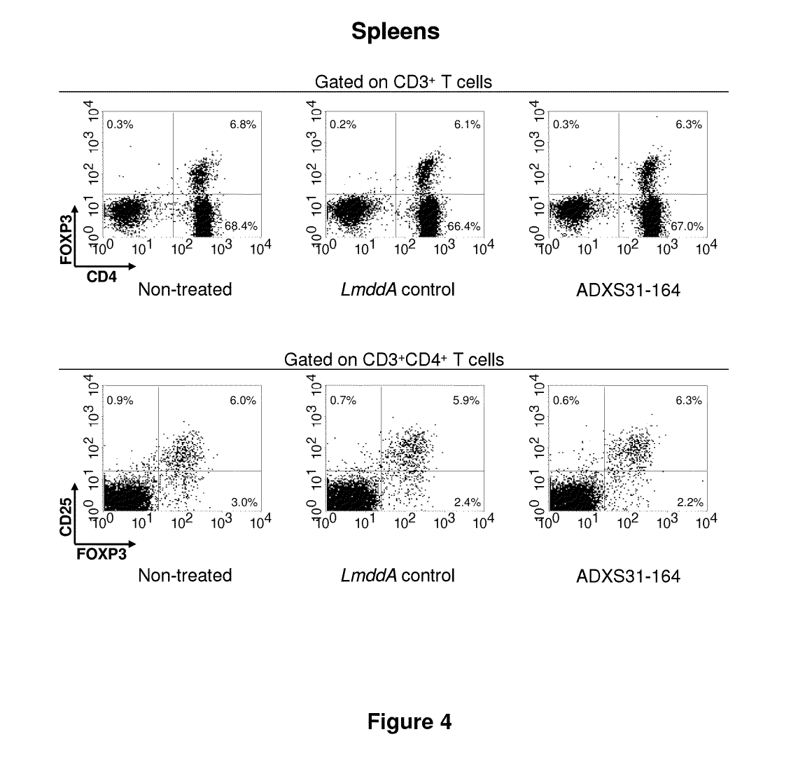

To elucidate the effect of ADXS31-164 on the frequency of regulatory T cells in spleens and tumors, mice were implanted with NT-2 tumor cells. Splenocytes and intra-tumoral lymphocytes were isolated after three immunizations and stained for Tregs, which were defined as CD3⁺/CD4⁺/CD25⁺/FoxP3⁺ cells, although comparable results were obtained with either FoxP3 or CD25 markers when analyzed separately. The results indicated that immunization with ADXS31-164 had no effect on the frequency of Tregs in the spleens, as compared to an irrelevant Listeria vaccine or the naïve animals (See FIG. 4). In contrast, immunization with the Listeria vaccines caused a considerable impact on the presence of Tregs in the tumors (FIG. 5A). Whereas in average 19.0% of all CD3⁺ T cells in untreated tumors were Tregs, this frequency was reduced to 4.2% for the irrelevant vaccine and 3.4% for ADXS31-164, a 5-fold reduction in the frequency of intra-tumoral Tregs (FIG. 5B). The decrease in the frequency of intra-tumoral Tregs in mice treated with either of the LmddA vaccines could not be attributed to differences in the sizes of the tumors. In a representative experiment, the tumors from mice immunized with ADXS31-164 were significantly smaller [mean diameter (mm)±SD, 6.71±0.43, n=5] than the tumors from untreated mice (8.69±0.98, n=5, p<0.01) or treated with the irrelevant vaccine (8.41±1.47, n=5, p=0.04), whereas comparison of these last two groups showed no statistically significant difference in tumor size (p=0.73). The lower frequency of Tregs in tumors treated with LmddA vaccines resulted in an increased intratumoral CD8/Tregs ratio, suggesting that a more favorable tumor microenvironment can be obtained after immunization with LmddA vaccines. However, only the vaccine expressing the target antigen HER2/neu (ADXS31-164) was able to reduce tumor growth, indicating that the decrease in Tregs has an effect only in the presence on antigen-specific responses in the tumor.

### Example 6

### No Escape Mutations were Introduced by Listeria Vaccine Expressing Her-2 Chimera

Tumor samples of the mice immunized with different vaccines such as Lm-LLO-138, LmddA164 and irrelevant vaccine Lm-LLO-NY were harvested. The DNA was purified from these samples and the DNA fragments corresponding to Her-2/neu regions IC 1, EC 1 and EC2 were amplified and were sequenced to determine if there were any immune escape mutations. The alignment of sequence from each DNA was performed using CLUSTALW. The results of the analysis indicated that there were no mutations in the DNA sequences harvested from tumors. The detailed analysis of these sequences is shown below.

### Alignment of EC2 (975-1029 bp of Her-2-Neu)

Reference
(SEQ ID NO: 14)
GGTCACAGCTGAGGACGGAACACAGCGTTGTGAGAAATGCAGCAAGCCCTGTGCT
Lm-LLO-138-2
GGTCACAGCTGAGGACGGAACACAGCGTTGTGAGAAATGCAGCAAGCCCTGTGCT
Lm-LLO-138-3
GGTCACAGCTGAGGACGGAACACAGCGTTGTGAGAAATGCAGCAAGCCCTGTGCT
Lm-ddA-164-1
GGTCACAGCTGAGGACGGAACACAGCGTTGTGAGAAATGCAGCAAGCCCTGTGCT
LmddA164-2
GGTCACAGCTGAGGACGGAACACAGCGTTGTGAGAAATGCAGCAAGCCCTGTGCT
Lm-ddA-164-3
GGTCACAGCTGAGGACGGAACACAGCGTTGTGAGAAATGCAGCAAGCCCTGTGCT
LmddA164-4
GGTCACAGCTGAGGACGGAACACAGCGTTGTGAGAAATGCAGCAAGCCCTGTGCT
Lm-ddA-164-5
GGTCACAGCTGAGGACGGAACACAGCGTTGTGAGAAATGCAGCAAGCCCTGTGCT
LmddA-164-6
GGTCACAGCTGAGGACGGAACACAGCGTTCTGAGAAATGCAGCAAGCCCTGTGCT
Reference
(SEQ ID NO: 15)
CGAGTGTGCTATGGTCTGGGCATGGAGCACCTTCGAGGGGCGAGGGCCATCACCAGTGAC
Lm-LLO-138-2
CGAGTGTGCTATGGTCTGGGCATGGAGCACCTTCGAGGGGCGAGGGCCATCACCAGTGAC
Lm-LLO-138-3
CGAGTGTGCTATGGTCTGGGCATGGAGCACCTTCGAGGGGCGAGGGCCATCACCAGTGAC
Lm-ddA-164-1
CGAGTGTGCTATGGTCTGGGCATGGAGCACCTTCGAGGGGCGAGGGCCATCACCAGTGAC
LmddA164-2
CGAGTGTGCTATGGTCTGGGCATGGAGCACCTTCGAGGGGCGAGGGCCATCACCAGTGAC
Lm-ddA-164-3
CGAGTGTGCTATGGTCTGGGCATGGAGCACCTTCGAGGGGCGAGGGCCATCACCAGTGAC
LmddA164-4
CGAGTGTGCTATGGTCTGGGCATGGAGCACCTTCGAGGGGCGAGGGCCATCACCAGTGAC
Lm-ddA-164-5
CGAGTGTGCTATGGTCTGGGCATGGAGCACCTTCGAGGGGCGAGGGCCATCACCAGTGAC
LmddA-164-6
CGAGTGTGCTATGGTCTGGGCATGGAGCACCTTCGAGGGGCGAGGGCCATCACCAGTGAC
Reference
(SEQ ID No: 16)
AATGTCCAGGAGTTTGATGGCTGCAAGAAGATCTTTGGGAGCCTGGCATTTTTGCCGGAG
Lm-LLO-138-2
AATGTCCAGGAGTTTGATGGCTGCAAGAAGATCTTTGGGAGCCTGGCATTTTTGCCGGAG
Lm-LLO-138-3
AATGTCCAGGAGTTTGATGGCTGCAAGAAGATCTTTGGGAGCCTGGCATTTTTGCCGGAG
Lm-ddA-164-1
AATGTCCAGGAGTTTGATGGCTGCAAGAAGATCTTTGGGAGCCTGGCATTTTTGCCGGAG
LmddA164-2
AATGTCCAGGAGTTTGATGGCTGCAAGAAGATCTTTGGGAGCCTGGCATTTTTGCCGGAG
Lm-ddA-164-3
AATGTCCAGGAGTTTGATGGCTGCAAGAAGATCTTTGGGAGCCTGGCATTTTTGCCGGAG
LmddA164-4
AATGTCCAGGAGTTTGATGGCTGCAAGAAGATCTTTGGGAGCCTGGCATTTTTGCCGGAG
Lm-ddA-164-5
AATGTCCAGGAGTTTGATGGCTGCAAGAAGATCTTTGGGAGCCTGGCATTTTTGCCGGAG
LmddA-164-6
AATGTCCAGGAGTTTGATGGCTGCAAGAAGATCTTTGGGAGCCTGGCATTTTTGCCGGAG
Reference
(SEQ ID NO: 17)
AGCTTTGATGGGGACCCCTCCTCCGGCATTGCTCCGCTGAGGCCTGAGCAGCTCCAAGTG
Lm-LLO-138-2
AGCTTTGATGGGGACCCCTCCTCCGGCATTGCTCCGCTGAGGCCTGAGCAGCTCCAAGTG
Lm-LLO-138-3
AGCTTTGATGGGGACCCCTCCTCCGGCATTGCTCCGCTGAGGCCTGAGCAGCTCCAAGTG
Lm-ddA-164-1
AGCTTTGATGGGGACCCCTCCTCCGGCATTGCTCCGCTGAGGCCTGAGCAGCTCCAAGTG
LmddA164-2
AGCTTTGATGGGGACCCCTCCTCCGGCATTGCTCCGCTGAGGCCTGAGCAGCTCCAAGTG
Lm-ddA-164-3
AGCTTTGATGGGGACCCCTCCTCCGGCATTGCTCCGCTGAGGCCTGAGCAGCTCCAAGTG
LmddA164-4
AGCTTTGATGGGGACCCCTCCTCCGGCATTGCTCCGCTGAGGCCTGAGCAGCTCCAAGTG
Lm-ddA-164-5
AGCTTTGATGGGGACCCCTCCTCCGGCATTGCTCCGCTGAGGCCTGAGCAGCTCCAAGTG
LmddA-164-6
AGCTTTGATGGGGACCCCTCCTCCGGCATTGCTCCGCTGAGGCCTGAGCAGCTCCAAGTG
Reference
(SEQ ID NO: 18)
TTCGAAACCCTGGAGGAGATCACAGGTTACCTGTACATCTCAGCATGGCCAGACAGTCTC
Lm-LLO-138-2
TTCGAAACCCTGGAGGAGATCACAGGTTACCTGTACATCTCAGCATGGCCAGACAGTCTC
Lm-LLO-138-3
TTCGAAACCCTGGAGGAGATCACAGGTTACCTGTACATCTCAGCATGGCCAGACAGTCTC
Lm-ddA-164-1
TTCGAAACCCTGGAGGAGATCACAGGTTACCTGTACATCTCAGCATGGCCAGACAGTCTC
LmddA164-2
TTCGAAACCCTGGAGGAGATCACAGGTTACCTGTACATCTCAGCATGGCCAGACAGTCTC
Lm-ddA-164-3
TTCGAAACCCTGGAGGAGATCACAGGTTACCTGTACATCTCAGCATGGCCAGACAGTCTC
LmddA164-4
TTCGAAACCCTGGAGGAGATCACAGGTTACCTGTACATCTCAGCATGGCCAGACAGTCTC
Lm-ddA-164-5
TTCGAAACCCTGGAGGAGATCACAGGTTACCTGTACATCTCAGCATGGCCANACAGTCTC
LmddA-164-6
TTCGAAACCCTGGAGGAGATCACAGGTTACCTGTACATCTCAGCATGGCCAGACAGTCT
Reference
(SEQ ID NO: 19)
CGTGACCTCAGTGTCTTCCAGAACCTTCGAATCATTCGGGGACGGATTCTCCACGATGGC
Lm-LLO-138-2
CGTGACCTCAGTGTCTTCCAGAACCTTCGAATCATTCGGGGACGGATTCTCCACGATGGC
Lm-LLO-138-3
CGTGACCTCAGTGTCTTCCAGAACCTTCGAATCATTCGGGGACGGATTCTCCACGATGGC
Lm-ddA-164-1
CGTGACCTCAGTGTCTTCCAGAACCTTCGAATCATTCGGGGACGGATTCTCCACGATGGC
LmddA164-2
CGTGACCTCAGTGTCTTCCAGAACCTTCGAATCATTCGGGGACGGATTCTCCACGATGGC
Lm-ddA-164-3
CGTGACCTCAGTGTCTTCCAGAACCTTCGAATCATTCGGGGACGGATTCTCCACGATGGC
LmddA164-4
CGTGACCTCAGTGTCTTCCAAAACCTTCGAATCATTCGGGGACGGATTCTCCACGATGGC
Lm-ddA-164-5
CGTGACCTCAGTGTCTTCCAAAACCTTCGAATCATTCGGGGACGGATTCTCCACGATGGC
LmddA-164-6
CGTGACCTCAGTGTCTTCCAAAACCTTCGAATCATTCGGGGACGGATTCTCCACGATGGC
Reference
(SEQ ID NO: 20)
GCGTACTCATTGACACTGCAAGGCCTGGGGATCCACTCGCTGGGGCTGCGCTCACTGCGG
Lm-LLO-138-2
GCGTACTCATTGACACTGCAAGGCCTGGGGATCCACTCGCTGGGGCTGCGCTCACTGCGG
Lm-LLO-138-3
GCGTACTCATTGACACTGCAAGGCCTGGGGATCCACTCGCTGGGGCTGCGCTCACTGCGG
Lm-ddA-164-1
GCGTACTCATTGACACTGCAAGGCCTGGGGATCCACTCGCTGGGGCTGCGCTCACTGCGG
LmddA164-3
GCGTACTCATTGACACTGCAAGGCCTGGGGATCCACTCGCTGGGGCTGCGCTCACTGCGG
Lm-ddA-164-5
GCGTACTCATTGACACTGCAAGGCCTGGGGATCCACTCGCTGGGGCTGCGCTCACTGCGG
Lm-ddA-164-6
GCGTACTCATTGACACTGCAAGGCCTGGGGATCCACTCGCTGGGGCTGCGCTCACTGCGG
Reference
(SEQ ID NO: 21)
GAGCTGGGCAGTGGATTGGCTCTGATTCACCGCAACGCCCATCTCTGCTTTGTACACACT
Lm-LLO-138-2
GAGCTGGGCAGTGGATTGGCTCTGATTCACCGCAACGCCCATCTCTGCTTTGTACACACT
Lm-LLO-138-3
GAGCTGGGCAGTGGATTGGCTCTGATTCACCGCAACGCCCATCTCTGCTTTGTACACACT
Lm-ddA-164-1
GAGCTGGGCAGTGGATTGGCTCTGATTCACCGCAACGCCCATCTCTGCTTTGTACACACT
LmddA164-3
GAGCTGGGCAGTGGATTGGCTCTGATTCACCGCAACGCCCATCTCTGCTTTGTACACACT
Lm-ddA-164-5
GAGCTGGGCAGTGGATTGGCTCTGATTCACCGCAACGCCCATCTCTGCTTTGTACACACT
Lm-ddA-164-6
GAGCTGGGCAGTGGATTGGCTCTGATTCACCGCAACGCCCATCTCTGCTTTGTACACACT
Reference
(SEQ ID NO: 22)
GTACCTTGGGACCAGCTCTTCCGGAACCCACATCAGGCCCTGCTCCACAGTGGGAACCGG
Lm-LLO-138-2
GTACCTTGGGACCAGCTCTTCCGGAACCCACATCAGGCCCTGCTCCACAGTGGGAACCGG
Lm-LLO-138-3
GTACCTTGGGACCAGCTCTTCCGGAACCCACATCAGGCCCTGCTCCACAGTGGGAACCGG
Lm-ddA-164-1
GTACCTTGGGACCAGCTCTTCCGGAACCCACATCAGGCCCTGCTCCACAGTGGGAACCGG
LmddA164-3
GTACCTTGGGACCAGCTCTTCCGGAACCCACATCAGGCCCTGCTCCACAGTGGGAACCGG
Lm-ddA-164-5
GTACCTTGGGACCANCTCTTCCGGAACCCACATCAGGCCCTGCTCCACAGTGGGAACCGG
Lm-ddA-164-6
GTACCTTGGGACCAGCTCTTCCGGAACCCACATCAGGCCCTGCTCCACAGTGGGAACCGG
Reference
(SEQ ID NO: 23)
CCGGAAGAGGATTGTGGTCTCGAGGGCTTGGTCTGTAACTCACTGTGTGCCCACGGGCAC
Lm-LLO-138-2
CCGGAAGAGGATTGTGGTCTCGAGGGCTTGGTCTGTAACTCACTGTGTGCCCACGGGCAC
Lm-LLO-138-3
CCGGAAGAGGATTGTGGTCTCGAGGGCTTGGTCTGTAACTCACTGTGTGCCCACGGGCAC
Lm-ddA-164-1
CCGGAAGAGGATTGTGGTCTCGAGGGCTTGGTCTGTAACTCACTGTGTGCCCACGGGCAC
LmddA164-3
CCGGAAGAGGATTGTGGTCTCGAGGGCTTGGTCTGTAACTCACTGTGTGCCCACGGGCAC
Lm-ddA-164-6
CCGGAAGAGGATTGTGGTCTCGAGGGCTTGGTCTGTAACTCACTGTGTGCCCACGGGCAC
Reference
(SEQ ID NO: 24)
TGCTGGGGGCCAGGGCCCACCCAGTGTGTCAACTGCAGTCATTTCCTTCGGGGCCAGGAG
Lm-LLO-138-2
TGCTGGGGGCCAGGGCCCACCCAGTGTGTCAACTGCAGTCATTTCCTTCGGGGCCAGGAG
Lm-LLO-138-3
TGCTGGGGGCCAGGGCCCACCCAGTGTGTCAACTGCAGTCATTTCCTTCGGGGCCAGGAG
Lm-ddA-164-1
TGCTGGGGGCCAGGGCCCACCCAGTGTGTCAACTGCAGTCATTTCCTTCGGGGCCAGGAG
LmddA164-3
TGCTGGGGGCCAGGGCCCACCCAGTGTGTCAACTGCAGTCATTTCCTTCGGGGCCAGGAG
Lm-ddA-164-6
TGCTGGGGGCCAGGGCCCACCCA-------------------------------------
Alignment of IC1 (2114-3042 bp of Her-2-neu)
Reference
(SEQ ID NO: 25)
CGCCCAGCGGAGCAATGCCCAACCAGGCTCAGATGCGGATCCTAAAAGAGACGGAGC
Lm-LLO-NY-2
CGCCCAGCGGAGCAATGCCCAACCAGGCTCAGATGCGGATCCTAAAAGAGACGGAGC
Lm-LLO-138-4
CGCCCAGCGGAGCAATGCCCAACCAGGCTCAGATGCGGATCCTAAAAGAGACGGAGC
Lm-ddA-164-2
CGCCCAGCGGAGCAATGCCCAACCAGGCTCAGATGCGGATCCTAAAAGAGACGGAGC
Lm-ddA-164-3
CGCCCAGCGGAGCAATGCCCAACCAGGCTCAGATGCGGATCCTAAAAGAGACGGAGC
Lm-ddA164-6
CGCCCAGCGGAGCAATGCCCAACCAGGCTCAGATGCGGATCCTAAAAGAGACGGAGC
Reference
(SEQ ID NO: 26)
TAAGGAAGGTGAAGGTGCTTGGATCAGGAGCTTTTGGCACTGTCTACAAGGGCATCTGGA
Lm-LLO-NY-1
TAAGGAAGGTGAAGGTGCTTGGATCAGGAGCTTTTGGCACTGTCTACAAGGGCATCTGGA
Lm-LLO-NY-2
TAAGGAAGGTGAAGGTGCTTGGATCAGGAGCTTTTGGCACTGTCTACAAGGGCATCTGGA
Lm-LLO-138-1
TAAGGAAGGTGAACGTGCTTGGATCAGGAGCTTTTGGCACTGTCTACAAGGGCATCTGGA
Lm-LLO-138-2
TAAGGAAGGTGAAGGTGCTTGGATCAGGAGCTTTTGGCACTGTCTACAAGGGCATCTGGA
Lm-LLO-138-3
TAAGGAAGGTGAAGGTGCTTGGATCAGGAGCTTTTGGCACTGTCTACAAGGGCATCTGGA
Lm-LLO-138-4
TAAGGAAGGTGAAGGTGCTTGGATCAGGAGCTTTTGGCACTGTCTACAAGGGCATCTGGA
Lm-ddA-164-1
TAAGGAAGGTGAAGGTGCTTGGATCAGGAGCTTTTGGCACTGTCTACAAGGGCATCTGGA
Lm-ddA-164-2
TAAGGAAGGTGAAGGTGCTTGGATCAGGAGCTTTTGGCACTGTCTACAAGGGCATCTGGA
Lm-ddA-164-3
TAAGGAAGGTGAAGGTGCTTGGATCAGGAGCTTTTGGCACTGTCTACAAGGGCATCTGGA
Lm-ddA-164-4
TAAGGAAGGTGAAGGTGCTTGGATCAGGAGCTTTTGGCACTGTCTACAAGGGCATCTGGA
Lm-ddA-164-5
TAAGGAAGGTGAAGGTGCTTGGATCAGGAGCTTTTGGCACTGTCTACAAGGGCATCTGGA
Lm-ddA164-6
TAAGGAAGGTGAAGGTGCTTGGATCAGGAGCTTTTGGCACTGTCTACAAGGGCATCTGGA
Reference
(SEQ ID NO: 27)
TCCCAGATGGGGAGAATGTGAAAATCCCCGTGGCTATCAAGGTGTTGAGAGAAAACACAT
Lm-LLO-NY-1
TCCCAGATGGGGAGAATGTGAAAATCCCCGTGGCTATCAAGGTGTTGAGAGAAAACACAT
Lm-LLO-NY-2
TCCCAGATGGGGAGAATGTGAAAATCCCCGTGGCTATCAAGGTGTTGAGAGAAAACACAT
Lm-LLO-138-1
TCCCAGATGGGGAGAATGTGAAAATCCCCGTGGCTATCAAGGTGTTGAGAGAAAACACAT
Lm-LLO-138-2
TCCCAGATGGGGAGAATGTGAAAATCCCCGTGGCTATCAAGGTGTTGAGAGAAAACACAT
Lm-LLO-138-3
TCCCAGATGGGGAGAATGTGAAAATCCCCGTGGCTATCAAGGTGTTGAGAGAAAACACAT
Lm-LLO-138-4
TCCCAGATGGGGAGAATGTGAAAATCCCCGTGGCTATCAAGGTGTTGAGAGAAAACACAT
Lm-ddA-164-1
TCCCAGATGGGGAGAATGTGAAAATCCCCGTGGCTATCAAGGTGTTGAGAGAAAACACAT
Lm-ddA-164-2
TCCCAGATGGGGAGAATGTGAAAATCCCCGTGGCTATCAAGGTGTTGAGAGAAAACACAT
Lm-ddA-164-3
TCCCAGATGGGGAGAATGTGAAAATCCCCGTGGCTATCAAGGTGTTGAGAGAAAACACAT
Lm-ddA-164-4
TCCCAGATGGGGAGAATGTGAAAATCCCCGTGGCTATCAAGGTGTTGAGAGAAAACACAT
Lm-ddA-164-5
TCCCAGATGGGGAGAATGTGAAAATCCCCGTGGCTATCAAGGTGTTGAGAGAAAACACAT
Lm-ddA164-6
TCCCAGATGGGGAGAATGTGAAAATCCCCGTGGCTATCAAGGTGTTGAGAGAAAACACAT
Reference
(SEQ ID NO: 28)
CTCCTAAAGCCAACAAAGAAATTCTAGATGAAGCGTATGTGATGGCTGGTGTGGGTTCTC
Lm-LLO-NY-1
CTCCTAAAGCCAACAAAGAAATTCTAGATGAAGCGTATGTGATGGCTGGTGTGGGTTCTC
Lm-LLO-NY-2
CTCCTAAAGCCAACAAAGAAATTCTAGATGAAGCGTATGTGATGGCTGGTGTGGGTTCTC
Lm-LLO-138-1
CTCCTAAAGCCAACAAAGAAATTCTAGATGAAGCGTATGTGATGGCTGGTGTGGGTTCTC
Lm-LLO-138-2
CTCCTAAAGCCAACAAAGAAATTCTAGATGAAGCGTATGTGATGGCTGGTGTGGGTTCTC
Lm-LLO-138-3
CTCCTAAAGCCAACAAAGAAATTCTAGATGAAGCGTATGTGATGGCTGGTGTGGGTTCTC
lm-LLO-138-4
CTCCTAAAGCCAACAAAGAAATTCTAGATGAAGCGTATGTGATGGCTGGTGTGGGTTCTC
Lm-ddA-164-1
CTCCTAAAGCCAACAAAGAAATTCTAGATGAAGCGTATGTGATGGCTGGTGTGGGTTCTC
Lm-ddA-164-2
CTCCTAAAGCCAACAAAGAAATTCTAGATGAAGCGTATGTGATGGCTGGTGTGGGTTCTC
Lm-ddA-164-3
CTCCTAAAGCCAACAAAGAAATTCTAGATGAAGCGTATGTGATGGCTGGTGTGGGTTCTC
Lm-ddA-164-4
CTCCTAAAGCCAACAAAGAAATTCTAGATGAAGCGTATGTGATGGCTGGTGTGGGTTCTC
Lm-ddA-164-5
CTCCTAAAGCCAACAAAGAAATTCTAGATGAAGCGTATGTGATGGCTGGTGTGGGTTCTC
Lm-ddA164-6
CTCCTAAAGCCAACAAAGAAATTCTAGATGAAGCGTATGTGATGGCTGGTGTGGGTTCTC
Reference
(SEQ ID NO: 29)
CGTATGTGTCCCGCCTCCTGGGCATCTGCCTGACATCCACAGTACAGCTGGTGACACAGC
Lm-LLO-NY-1
CGTATGTGTCCCGCCTCCTGGGCATCTGCCTGACATCCACAGTACAGCTGGTGACACAGC
Lm-LLO-NY-2
CGTATGTGTCCCGCCTCCTGGGCATCTGCCTGACATCCACAGTACAGCTGGTGACACAGC
Lm-LLO-138-1
CGTATGTGTCCCGCCTCCTGGGCATCTGCCTGACATCCACAGTACAGCTGGTGACACAGC
Lm-LLO-138-2
CGTATGTGTCCCGCCTCCTGGGCATCTGCCTGACATCCACAGTACAGCTGGTGACACAGC
Lm-LLO-138-3
CGTATGTGTCCCGCCTCCTGGGCATCTGCCTGACATCCACAGTACAGCTGGTGACACAGC
Lm-LLO-138-4
CGTATGTGTCCCGCCTCCTGGGCATCTGCCTGACATCCACAGTACAGCTGGTGACACAGC
Lm-ddA-164-1
CGTATGTGTCCCGCCTCCTGGGCATCTGCCTGACATCCACAGTACAGCTGGTGACACAGC
Lm-ddA-164-2
CGTATGTGTCCCGCCTCCTGGGCATCTGCCTGACATCCACAGTACAGCTGGTGACACAGC
Lm-ddA-164-3
CGTATGTGTCCCGCCTCCTGGGCATCTGCCTGACATCCACAGTACAGCTGGTGACACAGC
Lm-ddA-164-4
CGTATGTGTCCCGCCTCCTGGGCATCTGCCTGACATCCACAGTACAGCTGGTGACACAGC
Lm-ddA-164-5
CGTATGTGTCCCGCCTCCTGGGCATCTGCCTGACATCCACAGTACAGCTGGTGACACAGC
Lm-ddA164-6
CGTATGTGTCCCGCCTCCTGGGCATCTGCCTGACATCCACAGTACAGCTGGTGACACAGC
Reference
(SEQ ID NO: 30)
TTATGCCCTACGGCTGCCTTCTGGACCATGTCCGAGAACACCGAGGTCGCCTAGGCTCCC
Lm-LLO-NY-1
TTATGCCCTACGGCTGCCTTCTGGACCATGTCCGAGAACACCGAGGTCGCCTAGGCTCCC
Lm-LLO-NY-2
TTATGCCCTACGGCTGCCTTCTGGACCATGTCCGAGAACACCGAGGTCGCCTAGGCTCCC
Lm-LLO-138-1
TTATGCCCTACGGCTGCCTTCTGGACCATGTCCGAGAACACCGAGGTCGCCTAGGCTCCC
Lm-LLO-138-2
TTATGCCCTACGGCTGCCTTCTGGACCATGTCCGAGAACACCGAGGTCGCCTAGGCTCCC
Lm-LLO-138-3
TTATGCCCTACGGCTGCCTTCTGGACCATGTCCGAGAACACCGAGGTCGCCTAGGCTCCC
Lm-LLO-138-4
TTATGCCCTACGGCTGCCTTCTGGACCATGTCCGAGAACACCGAGGTCGCCTAGGCTCCC
Lm-ddA-164-1
TTATGCCCTACGGCTGCCTTCTGGACCATGTCCGAGAACACCGAGGTCGCCTAGGCTCCC
Lm-ddA-164-2
TTATGCCCTACGGCTGCCTTCTGGACCATGTCCGAGAACACCGAGGTCGCCTAGGCTCCC
Lm-ddA-164-3
TTATGCCCTACGGCTGCCTTCTGGACCATGTCCGAGAACACCGAGGTCGCCTAGGCTCCC
Lm-ddA-164-4
TTATGCCCTACGGCTGCCTTCTGGACCATGTCCGAGAACACCGAGGTCGCCTAGGCTCCC
Lm-ddA-164-5
TTATGCCCTACGGCTGCCTTCTGGACCATGTCCGAGAACACCGAGGTCGCCTAGGCTCCC
Lm-ddA164-6
TTATGCCCTACGGCTGCCTTCTGGACCATGTCCGAGAACACCGAGGTCGCCTAGGCTCCC
Reference
(SEQ ID NO: 31)
AGGACCTGCTCAACTGGTGTGTTCAGATTGCCAAGGGGATGAGCTACCTGGAGGACGTGC
Lm-LLO-NY-1
AGGACCTGCTCAACTGGTGTGTTCAGATTGCCAAGGGGATGAGCTACCTGGAGGACGTGC
Lm-LLO-NY-2
AGGACCTGCTCAACTGGTGTGTTCAGATTGCCAAGGGGATGAGCTACCTGGAGGACGTGC
Lm-LLO-138-1
AGGACCTGCTCAACTGGTGTGTTCAGATTGCCAAGGGGATGAGCTACCTGGAGGACGTGC
Lm-LLO-138-2
AGGACCTGCTCAACTGGTGTGTTCAGATTGCCAAGGGGATGAGCTACCTGGAGGACGTGC
Lm-LLO-138-3
AGGACCTGCTCAACTGGTGTGTTCAGATTGCCAAGGGGATGAGCTACCTGGAGGACGTGC
Lm-LLO-138-4
AGGACCTGCTCAACTGGTGTGTTCAGATTGCCAAGGGGATGAGCTACCTGGAGGACGTGC
Lm-ddA-164-1
AGGACCTGCTCAACTGGTGTGTTCAGATTGCCAAGGGGATGAGCTACCTGGAGGACGTGC
Lm-ddA-164-2
AGGACCTGCTCAACTGGTGTGTTCAGATTGCCAAGGGGATGAGCTACCTGGAGGACGTGC
Lm-ddA-164-3
AGGACCTGCTCAACTGGTGTGTTCAGATTGCCAAGGGGATGAGCTACCTGGAGGACGTGC
Lm-ddA-164-4
AGGACCTGCTCAACTGGTGTGTTCAGATTGCCAAGGGGATGAGCTACCTGGAGGACGTGC
Lm-ddA-164-5
AGGACCTGCTCAACTGGTGTGTTCAGATTGCCAAGGGGATGAGCTACCTGGAGGACGTGC
Lm-ddA164-6
AGGACCTGCTCAACTGGTGTGTTCAGATTGCCAAGGGGATGAGCTACCTGGAGGACGTGC
Reference
(SEQ ID NO: 32)
GGCTTGTACACAGGGACCTGGCTGCCCGGAATGTGCTAGTCAAGAGTCCCAACCACGTCA
Lm-LLO-NY-1
GGCTTGTACACAGGGACCTGGCTGCCCGGAATGTGCTAGTCAAGAGTCCCAACCACGTCA
Lm-LLO-NY-2
GGCTTGTACACAGGGACCTGGCTGCCCGGAATGTGCTAGTCAAGAGTCCCAACCACGTCA
Lm-LLO-138-1
GGCTTGTACACAGGGACCTGGCTGCCCGGAATGTGCTAGTCAAGAGTCCCAACCACGTCA
Lm-LLO-138-2
GGCTTGTACACAGGGACCTGGCTGCCCGGAATGTGCTAGTCAAGAGTCCCAACCACGTCA
Lm-LLO-138-3
GGCTTGTACACAGGGACCTGGCTGCCCGGAATGTGCTAGTCAAGAGTCCCAACCACGTCA
Lm-LLO-138-4
GGCTTGTACACAGGGACCTGGCTGCCCGGAATGTGCTAGTCAAGAGTCCCAACCACGTCA
Lm-ddA-164-1
GGCTTGTACACAGGGACCTGGCTGCCCGGAATGTGCTAGTCAAGAGTCCCAACCACGTCA
Lm-ddA-164-2
GGCTTGTACACAGGGACCTGGCTGCCCGGAATGTGCTAGTCAAGAGTCCCAACCACGTCA
Lm-ddA-164-4
GGCTTGTACACAGGGACCTGGCTGCCCGGAATGTGCTAGTCAAGAGTCCCAACCACGTCA
Lm-ddA-164-3
GGCTTGTACACAGGGACCTGGCTGCCCGGAATGTGCTAGTCAAGAGTCCCAACCACGTCA
Lm-ddA-164-5
GGCTTGTACACAGGGACCTGGCTGCCCGGAATGTGCTAGTCAAGAGTCCCAACCACGTCA
Lm-ddA164-6
GGCTTGTACACAGGGACCTGGCTGCCCGGAATGTGCTAGTCAAGAGTCCCAACCACGTCA
Reference
(SEQ ID NO: 33)
AGATTACAGATTTCGGGCTGGCTCGGCTGCTGGACATTGATGAGACAGAGTACCATGCAG
Lm-LLO-NY-1
AGATTACAGATTTCGGGCTGGCTCGGCTGCTGGACATTGATGAGACAGAGTACCATGCAG
Lm-LLO-NY-2
AGATTACAGATTTCGGGCTGGCTCGGCTGCTGGACATTGATGAGACAGAGTACCATGCAG
Lm-LLO-138-1
AGATTACAGATTTCGGGCTGGCTCGGCTGCTGGACATTGATGAGACAGAGTACCATGCAG
Lm-LLO-138-2
AGATTACAGATTTCGGGCTGGCTCGGCTGCTGGACATTGATGAGACAGAGTACCATGCAG
Lm-LLO-138-3
AGATTACAGATTTCGGGCTGGCTCGGCTGCTGGACATTGATGAGACAGAGTACCATGCAG
Lm-LLO-138-4
AGATTACAGATTTCGGGCTGGCTCGGCTGCTGGACATTGATGAGACAGAGTACCATGCAG
Lm-ddA-164-1
AGATTACAGATTTCGGGCTGGCTCGGCTGCTGGACATTGATGAGACAGAGTACCATGCAG
Lm-ddA-164-2
AGATTACAGATTTCGGGCTGGCTCGGCTGCTGGACATTGATGAGACAGAGTACCATGCAG
Lm-ddA-164-3
AGATTACAGATTTCGGGCTGGCTCGGCTGCTGGACATTGATGAGACAGAGTACCATGCAG
Lm-ddA-164-4
AGATTACAGATTTCGGGCTGGCTCGGCTGCTGGACATTGATGAGACAGAGTACCATGCAG
Lm-ddA-164-5
AGATTACAGATTTCGGGCTGGCTCGGCTGCTGGACATTGATGAGACAGAGTACCATGCAG
Lm-ddA164-6
AGATTACAGATTTCGGGCTGGCTCGGCTGCTGGACATTGATGAGACAGAGTACCATGCAG
Reference
(SEQ ID NO: 34)
ATGGGGGCAAGGTGCCCATCAAATGGATGGCATTGGAATCTATTCTCAGACGCCGGTTCA
Lm-LLO-NY-1
ATGGGGGCAAGGTGCCCATCAAATGGATGGCATTGGAATCTATTCTCAGACGCCGGTTCA
Lm-LLO-NY-2
ATGGGGGCAAGGTGCCCATCAAATGGATGGCATTGGAATCTATTCTCAGACGCCGGTTCA
Lm-LLO-138-1
ATGGGGGCAAGGTGCCCATCAAATGGATGGCATTGGAATCTATTCTCAGACGCCGGTTCA
Lm-LLO-138-2
ATGGGGGCAAGGTGCCCATCAAATGGATGGCATTGGAATCTATTCTCAGACGCCGGTTCA
Lm-LLO-138-3
ATGGGGGCAAGGTGCCCATCAAATGGATGGCATTGGAATCTATTCTCAGACGCCGGTTCA
Lm-LLO-138-4
ATGGGGGCAAGGTGCCCATCAAATGGATGGCATTGGAATCTATTCTCAGACGCCGGTTCA
Lm-ddA-164-1
ATGGGGGCAAGGTGCCCATCAAATGGATGGCATTGGAATCTATTCTCAGACGCCGGTTCA
Lm-ddA-164-2
ATGGGGGCAAGGTGCCCATCAAATGGATGGCATTGGAATCTATTCTCAGACGCCGGTTCA
Lm-ddA-164-3
ATGGGGGCAAGGTGCCCATCAAATGGATGGCATTGGAATCTATTCTCAGACGCCGGTTCA
Lm-ddA-164-4
ATGGGGGCAAGGTGCCCATCAAATGGATGGCATTGGAATCTATTCTCAGACGCCGGTTCA
Lm-ddA-164-5
ATGGGGGCAAGGTGCCCATCAAATGGATGGCATTGGAATCTATTCTCAGACGCCGGTTCA
Lm-ddA-164-6
ATGGGGGCAAGGTGCCCATCAAATGGATGGCATTGGAATCTATTCTCAGACGCCGGTTCA
Reference
(SEQ ID NO: 35)
CCCATCAGAGTGATGTGTGGAGCTATGGAGTGACTGTGTGGGAGCTGATGACTTTTGGGG
Lm-LLO-NY-1
CCCATCAGAGTGATGTGTGGAGCTATGGAGTGACTGTGTGGGAGCTGATGACTTTTGGGG
Lm-LLO-NY-2
CCCATCAGAGTGATGTGTGGAGCTATGGAGTGACTGTGTGGGAGCTGATGACTTTTGGGG
Lm-LLO-138-1
CCCATCAGAGTGATGTGTGGAGCTATGGAGTGACTGTGTGGGAGCTGATGACTTTTGGGG
Lm-LLO-138-2
CCCATCAGAGTGATGTGTGGAGCTATGGAGTGACTGTGTGGGAGCTGATGACTTTTGGGG
Lm-LLO-138-3
CCCATCAGAGTGATGTGTGGAGCTATGGAGTGACTGTGTGGGAGCTGATGACTTTTGGGG
Lm-LLO-138-4
CCCATCAGAGTGATGTGTGGAGCTATGGAGTGACTGTGTGGGAGCTGATGACTTTTGGGG
Lm-ddA-164-1
CCCATCAGAGTGATGTGTGGAGCTATGGAGTGACTGTGTGGGAGCTGATGACTTTTGGGG
Lm-ddA-164-2
CCCATCAGAGTGATGTGTGGAGCTATGGAGTGACTGTGTGGGAGCTGATGACTTTTGGGG
Lm-ddA-164-3
CCCATCAGAGTGATGTGTGGAGCTATGGAGTGACTGTGTGGGAGCTGATGACTTTTGGGG
Lm-ddA-164-4
CCCATCAGAGTGATGTGTGGAGCTATGGAGTGACTGTGTGGGAGCTGATGACTTTTGGGG
Lm-ddA-164-5
CCCATCAGAGTGATGTGTGGAGCTATGGAGTGACTGTGTGGGAGCTGATGACTTTTGGGG
Lm-ddA164-6
CCCATCAGAGTGATGTGTGGAGCTATGGAGTGACTGTGTGGGAGCTGATGACTTTTGGGG
Reference
(SEQ ID NO: 36)
CCAAACCTTACGATGGAATCCCAGCCCGGGAGATCCCTGATTTGCTGGAGAAGGGAGAA
Lm-LLO-NY-1
CCAAACCTTACGATGGAATCCCAGCCCGGGAGATCCCTGATTTGCTGGAGAAGGGAGAA
Lm-LLO-NY-2
CCAAACCTTACGATGGAATCCCAGCCCGGGAGATCCCTGATTTGCTGGAGAAGGGAGAA
Lm-LLO-138-1
CCAAACCTTACGATGGAATCCCAGCCCGGGAGATCCCTGATTTGCTGGAGAAGGGAGAA
Lm-LLO-138-3
CCAAACCTTACGATGGAATCCCAGCCCGGGAGATCCCTGATTTGCTGGAGAAGGGAGAA
Lm-LLO-138-4
CCAAACCTTACGATGNAATCCCAGCCCGGGAGATCCCTGATTTGCTGGAGAAGGGAGAA
Lm-ddA164-6
CCAAACCTTACGATGGAATCCCAGCCCGGGAGATCCCTGATTTGCTGGAGAAGGGAGAA
Lm-ddA-164-2
CCAAACCTTACGATGGAATCCCAGCCCGGGAGATCCCTGATTTGCTGGAGAAGGGAGAA
Lm-LLO-138-2
CCAAACCTTACGATGGAATCCCAGCCCGGGAGATCCCTGATTTGCTGGAGAAGGGAGAA
Lm-ddA-164-3
CCAAACCTTACGATGGAATCCCAGCCCGGGAGATCCCTGATTTGCTGGAGAAGGGAGAA
Lm-ddA-164-5
CCAAACCTTACGATGGAATCCCAGCCCGGGAGATCCCTGATTTGCTGGAGAAGGGAGAA
Lm-ddA-164-1
CCAAACCTTACGATGGAATCCCAGCCCGGGAGATCCCTGATTTGCTGGAGAAGGGAGAA
Lm-ddA-164-4
CCAAACCTTACGATGGAATCCCAGCCCGGGAGATCCCTGATTTGCTGGAGAAGGGAGAA
Reference
(SEQ ID NO: 37)
CGCCTACCTCAGCCTCCAATCTGCACCATTGATGTCTACATGATTATGGTCAAATGTT
Lm-LLO-NY-1
CGCCTACCTCAGCCTCCAATCTGCACCATTGATGTCTACATGATTATGGTCAAATGTT
Lm-LLO-NY-2
CGCCTACCTCAGCCTCCAATCTGCACCATTGATGTCTACATGATTATGGTCAAATGTT
Lm-LLO-138-1
CGCCTACCTCAGCCTCCAATCTGCACCATTGATGTCTACATGATTATGGTCAAATGTT
Lm-LLO-138-2
CGCCTACCTCAGCCTCCAATCTGCACCATTGATGTCTACATGATTATGGTCAAATGTT
Lm-LLO-138-3
CGCCTACCTCAGCCTCCAATCTGCACCATTGATGTCTACATGATTATGGTCAAATGTT
Lm-LLO-138-4
CGCCTACCTCAGCCTCCAATCTGCACCATTGATGTCTACATGATTATGGTCAAATGTT
Lm-ddA-164-1
CGCCTACCTCAGCCTCCAATCTGCACCATTGATGTCTACATGATTATGGTCAAATGTT
Lm-ddA-164-2
CGCCTACCTCAGCCTCCAATCTGCACCATTGATGTCTACATGATTATGGTCAAATGTT
Lm-ddA-164-3
CGCCTACCTCAGCCTCCAATCTGCACCATTGATGTCTACATGATTATGGTCAAATGTT
Lm-ddA-164-4
CGCCTACCTCAGCCTCCAATCTGCACCATTGATGTCTACATGATTATGGTCAAATGTT
Lm-ddA-164-5
CGCCTACCTCAGCCTCCAATCTGCACCATTGATGTCTACATGATTATGGTCAAATGTT
Lm-ddA164-6
CGCCTACCTCAGCCTCCAATCTGCACCATTGATGTCTACATGATTATGGTCAAATGTT
Reference
(SEQ ID NO: 38)
GGATGATTGACTCTGAATGTCGCCCGAGATTCCGGGAGTTGGTGTCAGAATTTT
Lm-LLO-NY-1
GGATGATTGACTCTGAATGTCGCCCGAGATTCCGGGAGTTGGTGTCAGAATTTT
Lm-LLO-NY-2
GGATGATTGACTCTGAATGTCGCCCGAGATTCCGGGAGTTGGTGTCAGAATTTT
Lm-LLO-138-2
GGATGATTGACTCTGAATGTCCCCCGAGATTCCGGGAGTTGGTGTCAAAATTTT
Lm-LLO-138-3
GGATGATTGACTCTGAATGTCGCCCGAGATTCCGGGAGTTGGTGTCAGAATTTT
Lm-LLO-138-4
GGATGATTGACTCTGAATGTCGCCCGAGATTCCGGGAGTTGGTGTCAGAATTTT
Lm-ddA-164-1
GGATGATTGACTCTGAATGTCGCCCGAGATTCCGGGAGTTGGTGTCAGAATTTT
Lm-ddA-164-2
GGATGATTGACTCTGAATGTCGCCCGAGATTCCGGGAGTTGGTGTCAGAATTTT
Lm-ddA-164-3
GGATGATTGACTCTGAATGTCGCCCGAGATTCCGGGAGTTGGTGTCAGAATTTT
Lm-ddA-164-5
GGATGATTGACTCTGAATGTCGCCCGAGATTCCGGGAGTTGGTGTCAGAATTTT
Lm-ddA-164-4
GGATGATTGACTCTGAATGTCGCCCGAGATTCCGGGAGTTGGTGTCAGAATTTT
Lm-ddA164-6
GGATGATTGACTCTGAATGTCGCCCGAGATTCCGGGAGTTGGTGTCAGAATTTT
Reference
(SEQ ID NO: 39)
CACGTATGGCGAGGGACCCCCAGCGTTTTGTGGTCATCCAGAACGAGGACTT
Lm-LLO-NY-1
CACGTATGGCGAGGGACCCCCAGCGTTTTGTGGTCATCCAGAACGAGGACTT
Lm-LLO-NY-2
CACGTATGGCGAGGGACCCCCAGCGTTTTGTGGTCATCCAGAACGAGGACTT
Lm-LLO-138-2
CACGTATGGCGAGGGACCCCCAGCGTTTTGTGGTCATCCAGAACGAGGACTT
Lm-LLO-138-3
CACGTATGGCGAGGGACCCCCAGCGTTTTGTGGTCATCCAGAACGAGGACTT
Lm-LLO-138-4
CACGTATGGCGAGGGACCCCCAGCGTTTTGTGGTCATCCAGAACGAGGACTT
Lm-ddA-164-1
CACGTATGGCGAGGGACCCCCAGCGTTTTGTGGTCATCCAGAACGAGGACTT
Lm-ddA-164-2
CACGTATGGCGAGGGACCCCCAGCGTTTTGTGGTCATCCAGAACGAGGACTT
Lm-ddA-164-3
CACGTATGGCGAGGGACCCCCAGCGTTTTGTGGTCATCCAGAACGAGGACTT
Lm-ddA-164-5
CACGTATGGCGAGGGACCCCCAGCGTTTTGTGGTCATCCAGAACGAGGACTT
Lm-ddA-164-6
CACGTATGGCGAGGGACCCCCAGCGTTTTGTGGTCATCCAGAACGAGGACTT

Alignment of EC1 (399-758 bp of Her-2-neu)

Reference
(SEQ ID NO: 40)
CCCAGGCAGAACCCCAGAGGGGCTGCGGGAGCTGCAGCTTCGAAGTCTCACAGAGATCCT
Lm-LLO-138-1
CCCAGGCAGAACCCCAGAGGGGCTGCGGGAGCTGCAGCTTCGAAGTCTCACAGAGATCCT
Lm-LLO-138-2
CCCAGGCAGAACCCCAGAGGGGCTGCGGGAGCTGCAGCTTCGAAGTCTCACAGAGATCCT
Lm-ddA-164-1
CCCAGGCAGAACCCCAGAGGGGCTGCGGGAGCTGCAGCTTCGAAGTCTCACAGAGATCCT
LmddA-164-2
CCCAGGCAGAACCCCAGAGGGGCTGCGGGAGCTGCAGCTTCGAAGTCTCACAGAGATCCT
LmddA-164-3
CCCAGGCAGAACCCCAGAGGGGCTGCGGGAGCTGCAGCTTCGAAGTCTCACAGAGATCCT
LmddA164-4
CCCAGGCAGAACCCCAGAGGGGCTGCGGGAGCTGCAGCTTCGAAGTCTCACAGAGATCCT
Reference
(SEQ ID NO: 41)
GAAGGGAGGAGTTTTGATCCGTGGGAACCCTCAGCTCTGCTACCAGGACATGGTTTTGTG
Lm-LLO-138-1
GAAGGGAGGAGTTTTGATCCGTGGGAACCCTCAGCTCTGCTACCAGGACATGGTTTTGTG
Lm-LLO-138-2
GAAGGGAGGAGTTTTGATCCGTGGGAACCCTCAGCTCTGCTACCAGGACATGGTTTTGTG
Lm-ddA-164-1
GAAGGGAGGAGTTTTGATCCGTGGGAACCCTCAGCTCTGCTACCAGGACATGGTTTTGTG
LmddA-164-2
GAAGGGAGGAGTTTTGATCCGTGGGAACCCTCAGCTCTGCTACCAGGACATGGTTTTGTG
LmddA-164-3
GAAGGGAGGAGTTTTGATCCGTGGGAACCCTCAGCTCTGCTACCAGGACATGGTTTTGTG
LmddA164-4
GAAGGGAGGAGTTTTGATCCGTGGGAACCCTCAGCTCTGCTACCAGGACATGGTTTTGTG
Reference
(SEQ ID NO: 42)
CCGGGCCTGTCCACCTTGTGCCCCCGCCTGCAAAGACAATCACTGTTGGGGTGAGAGTCC
Lm-LLO-138-1
CCGGGCCTGTCCACCTTGTGCCCCCGCCTGCAAAGACAATCACTGTTGGGGTGAGAGTCC
Lm-LLO-138-2
CCGGGCCTGTCCACCTTGTGCCCCCGCCTGCAAAGACAATCACTGTTGGGGTGAGAGTCC
Lm-ddA-164-1
CCGGGCCTGTCCACCTTGTGCCCCCGCCTGCAAAGACAATCACTGTTGGGGTGAGAGTCC
LmddA-164-2
CCGGGCCTGTCCACCTTGTGCCCCCGCCTGCAAAGACAATCACTGTTGGGGTGAGAGTCC
LmddA-164-3
CCGGGCCTGTCCACCTTGTGCCCCCGCCTGCAAAGACAATCACTGTTGGGGTGAGAGTCC
LmddA164-4
CCGGGCCTGTCCACCTTGTGCCCCCGCCTGCAAAGACAATCACTGTTGGGGTGAGAGTCC
Reference
(SEQ ID NO: 43)
GGAAGACTGTCAGATCTTGACTGGCACCATCTGTACCAGTGGTTGTGCCCGGTGCAAGGG
Lm-LLO-138-1
GGAAGACTGTCAGATCTTGACTGGCACCATCTGTACCAGTGGTTGTGCCCGGTGCAAGGG
Lm-LLO-138-2
GGAAGACTGTCAGATCTTGACTGGCACCATCTGTACCAGTGGTTGTGCCCGGTGCAAGGG
Lm-ddA-164-1
GGAAGACTGTCAGATCTTGACTGGCACCATCTGTACCAGTGGTTGTGCCCGGTGCAAGGG
LmddA-164-2
GGAAGACTGTCAGATCTTGACTGGCACCATCTGTACCAGTGGTTGTGCCCGGTGCAAGGG
LmddA-164-3
GGAAGACTGTCAGATCTTGACTGGCACCATCTGTACCAGTGGTTGTGCCCGGTGCAAGGG
LmddA164-4
GGAAGACTGTCAGATCTTGACTGGCACCATCTGTACCAGTGGTTGTGCCCGGTGCAAGGG
Reference
(SEQ ID NO: 44)
CCGGCTGCCCACTGACTGCTGCCATGAGCAGTGTGCCGCAGGCTGCACGGGCCCCAAGCA
Lm-LLO-138-1
CCGGCTGCCCACTGACTGCTGCCATGAGCAGTGTGCCGCAGGCTGCACGGGCCCCAAGCA
Lm-LLO-138-2
CCGGCTGCCCACTGACTGCTGCCATGAGCAGTGTGCCGCAGGCTGCACGGGCCCCAAGCA
Lm-ddA-164-1
CCGGCTGCCCACTGACTGCTGCCATGAGCAGTGTGCCGCAGGCTGCACGGGCCCCAAGCA
LmddA-164-2
CCGGCTGCCCACTGACTGCTGCCATGAGCAGTGTGCCGCAGGCTGCACGGGCCCCAAGTA
LmddA-164-3
CCGGCTGCCCACTGACTGCTGCCATGAGCAGTGTGCCGCAGGCTGCACGGGCCCCAAGTA
LmddA-164-4
CCGGCTGCCCACTGACTGCTGCCATGAGCAGTGTGCCGCAGGCTGCACGGGCCCCAAGTA

### Example 7

### Peripheral Immunization with ADXS31-164 can Delay the Growth of a Metastatic Breast Cancer Cell Line in the Brain

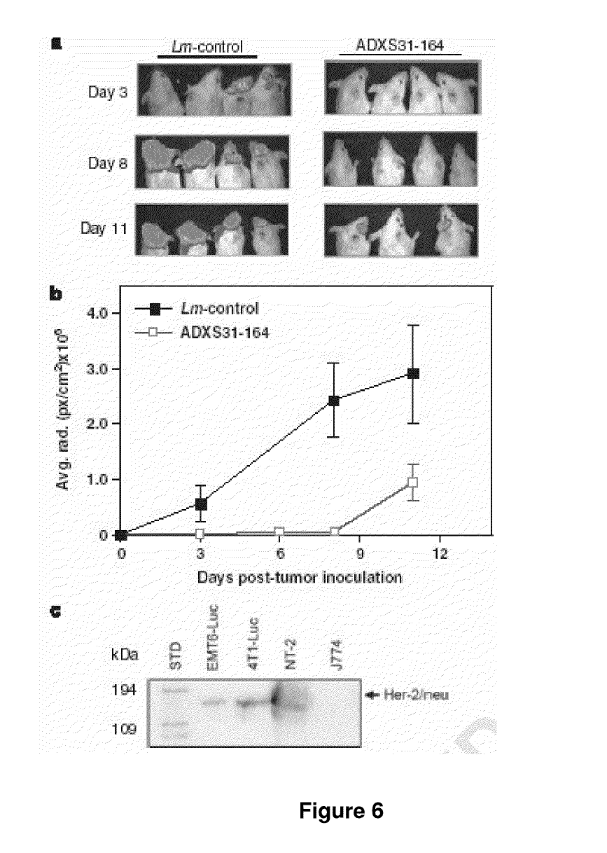

Mice were immunized IP with ADXS31-164 or irrelevant Lm-control vaccines and then implanted intra-cranially with 5,000 EMT6-Luc tumor cells, expressing luciferase and low levels of Her2/neu (FIG. 6C). Tumors were monitored at different times post-inoculation by ex vivo imaging of anesthetized mice. On day 8 post-tumor inoculation tumors were detected in all control animals, but none of the mice in ADXS31-164 group showed any detectable tumors (FIGS. 6A and B). ADXS31-164 could clearly delay the onset of these tumors, as on day 11 post-tumor inoculation all mice in negative control group had already succumbed to their tumors, but all mice in ADXS31-164 group were still alive and only showed small signs of tumor growth. These results strongly suggest that the immune responses obtained with the peripheral administration of ADXS31-164 could possibly reach the central nervous system and that LmddA-based vaccines might have a potential use for treatment of CNS tumors.

### Example 8

### Treatment of Canine Osteasarcoma by Immunization with ADXS31-164

Canine Osteosarcoma is a cancer of long (leg) bones that is a leading killer of large dogs over the age of 10 years. Standard treatment is amputation immediately after diagnosis, followed by chemotherapy. Invariably, however, the cancer metastasizes to the lungs. With chemotherapy, dogs survive about 18 months compared to 6-12 months, without treatment. The HER2 antigen is believed to be present in up to 50% of osteosarcoma. ADXS31-164 creates an immune attack on cells expressing this antigen and has been developed to treat human breast cancer.

Dogs with a histological diagnosis of osteosarcoma and evidence of expression of HER2/neu by malignant cells are eligible for enrollment.

### Canine Osteosarcoma Trial

In the first regiment the limbs are amputated, followed by round of chemotherapy treatment. 3 doses of Her-2 vaccine are subsequently administered with or without a 6 month interval booster.

All dogs are to receive 4 weeks of carboplatin therapy. Four weeks after the last carboplatin dose, dogs are to receive ADXS-HER2 once every three weeks for a total of 3 doses. Group 1 (3 dogs) receive 1×10⁸ CFU per dose, Group 2 (3 dogs) each receive 5×10⁸ CFU per dose and Group 3 (3 dogs) receives 1×10⁹ CFU per dose. Additional dogs are added to a Group to gather more data should if a potentially dose limiting toxicities, be observed. Therefore 9-18 dogs may be treated in the initial study.

In the second regiment, the same as the first regiment is repeated with the exception that only a single dose of vaccine is administered before chemotherapy (1 month before) for a total of 4 doses.

Further, in both regiments a single dose is administered a month after chemotherapy.

### Example 9

### Phase 1 Dose Escalation Study Evaluating the Safety of Adxs-Cher2 in Companion Dogs with Her2/Neu Overexpressing Canine Osteosarcoma

A pilot phase I dose escalation study was performed to determine the dose of a L. monocytogenes expressing human Her-2/neu recombinant vaccine that can safely and effectively stimulate tumor-specific immunity in dogs with osteosarcoma. The tumors of all dogs presenting to PennVet for limb amputation due to suspected or confirmed OSA were routinely harvested and evaluated histopathologically to confirm the diagnosis of OSA. In addition, tumor sections from all dogs were evaluated by IHC and Western blot analysis to determine whether the tumor expresses Her-2/neu. Only dogs with a histological diagnosis of OSA and evidence of expression of Her-2/neu by malignant cells were eligible for enrollment. Single cell suspensions of tumor tissue taken at surgery are cryopreserved and used as autologous tumor targets in chromium release assays to determine anti-tumor immunity.

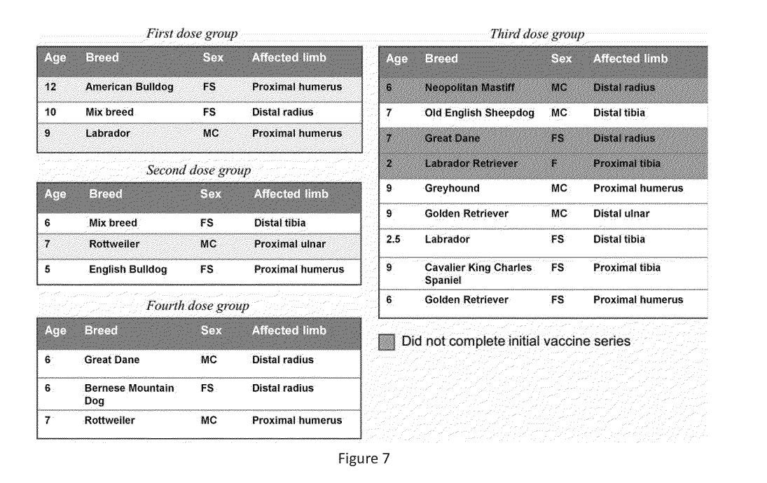

Up to 18 privately owned dogs with appendicular OSA and confirmed expression of Her2-neu were enrolled (FIG. 7). At enrollment (3 weeks post last carboplatin treatment), all dogs received basic clinical laboratory tests including a Complete Blood Count (CBC), Chemistry Screen (CS) and urinalysis (UA) and a baseline evaluation of cardiac function by echocardiography and measurement of cardiac-specific Troponin I (cTnI) levels. Thoracic radiographs are taken to determine whether pulmonary metastases are present. Only dogs with no evidence of pulmonary metastases were eligible for inclusion in the study. At the time of enrollment, peripheral blood mononuclear cells (PBMCs) are collected to assess baseline levels of anti-tumor immunity (see Assessment of anti-tumor immunity). Furthermore, blood was taken to evaluate baseline immune function to ensure they are no longer immune suppressed by carboplatin. Only dogs with functionally intact immune systems were eligible to receive the Listeria vaccine.

### Lm Recombinant Dosing and Data Capture

All dogs were vaccinated using a single Lm-huHer-2/neu recombinant vaccine. The first Lm-huHer2-neu vaccine were given three weeks after the last carboplatin dose and were given once every three weeks after this for a total of 3 doses (FIG. 7).

Group 1 (3 dogs) received the ADXS31-164 (Lm-hucHer-2/neu) vaccine at 1×10⁸ CFU per dose, Group 2 (3 dogs) each received 5×10⁸ CFU per dose, Group 3 (3 dogs) receive 1×10⁹ CFU per dose, and 3.3×10⁹ CFU per dose (1 dog). Recombinant Lm are administered as a slow intravenous infusion over 30 minutes. The dose chosen for Group 1 is the established safe dose for the chimeric huHer-2/neu recombinant in mice. In humans, the non-toxic dose for Lovaxin C is only one log higher than that established in mice, and this dose is the dose evaluated in Group 3 in this pilot trial.

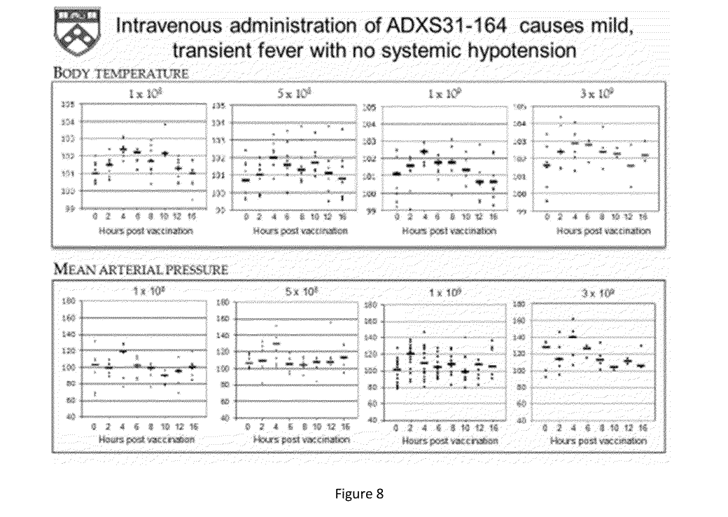

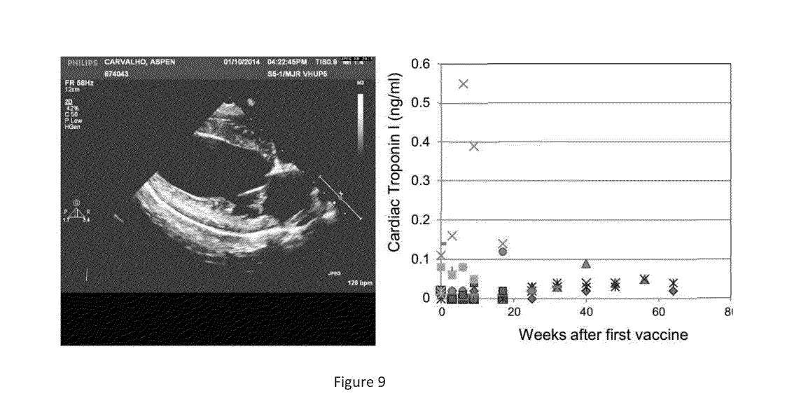

At the time of Lm administration, dogs were monitored for evidence of systemic adverse effects. During infusion, heart rate and rhythm was monitored by ECG and respiratory rate are recorded. Further, heart damage was monitored using ultrasound and by measuring Troponin I levels (FIG. 9). Following infusion, dogs are monitored closely for 48 hours. Core body temperature is monitored continuously for <12 hours post infusion using the Vital Sense continuous body temperature monitoring system by MiniMitter Respironics (routinely used in our Veterinary Clinical Trials Center, VCIC). Pulse rate, rhythm and quality, respiratory rate and effort, were monitored and recorded every hour for the first 6 hours then every 4 hours thereafter, as well as blood pressure and temperature (FIG. 8). All symptoms consistent with immune stimulation are noted and fluids, analgesics, anti-emetics and anti-histamines are used as necessary to control severe reactions. All dogs were observed six times a day and any signs of toxicological effects of the recombinants including discomfort, lethargy, nausea, vomiting and diarrhea were recorded. Blood samples were taken at 24, 48 and 72 hours after the first ADXS31-164 vaccine for cultures to assess the clearance of Lm after systemic administration.

### Assessment of Anti-Tumor Immunity

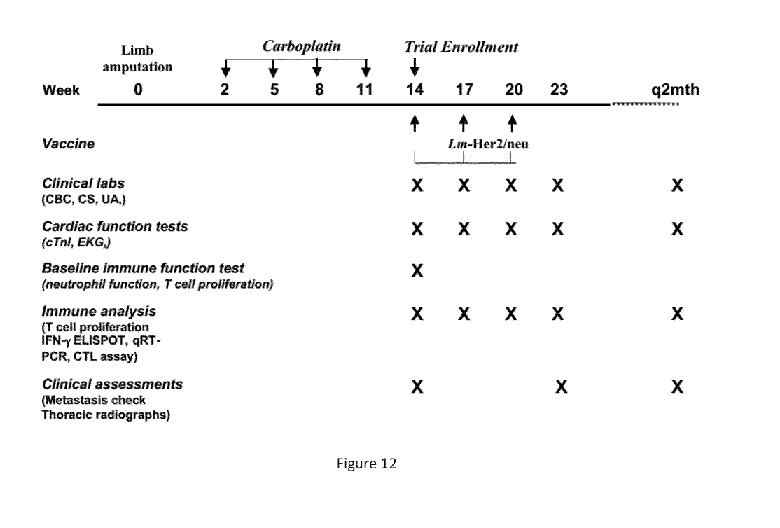

Three weeks following the last carboplatin dose, dogs receive a routine clinical examination and baseline blood work including CBC, CS, UA and cTnI levels. PBMCs are taken at this time for baseline evaluation of anti-tumor immunity. Repeat immune assessment is performed at the time of each vaccination and three weeks after the last vaccination. PBMCs are analyzed for Her-2/neu specific T cell responses by CFSE proliferation, cytokine production (ELISpot and qRT-PCR) and CTL assay against autologous tumor targets as outlined below (FIG. 12).

### Results

To date we have performed a total of 41 infusions of ADXS31-164 in 16 dogs.

| Number of | Number of | |
|---|---|---|
| dogs | infusions | Rationale |
| 1 | 5 | Two additional infusions post priming series to |
| | | treat metastatic disease |
| 4 | 4 | One additional infusion post priming series to |
| | | maintain tumor free status |
| 4 | 3 | Finished scheduled priming series |
| 1 | 2 | Succumbed to metastatic disease prior to finish of |
| | | priming course |
| 2 | 1 | Succumbed to metastatic disease prior to finish of |
| | | priming course |
| 4 | 1 | Priming course of vaccinations underway |

ADXS31-164 dose has ranged from 1×10⁸, 5×10⁸, 1×10⁹ and 3.3×10⁹ CFU.

| | Total number | Number | |
|---|---|---|---|
| Dose | of doses | of | |
| received | administered | dogs | Reported side effects |
| 1 × 10⁸ | 9 | 3 | Fever, nausea, vomiting, |
| | | | elevated liver enzymes |
| 5 × 10⁸ | 9 | 3 | Fever, nausea, vomiting, |
| | | | elevated liver enzymes |
| 1 × 10⁹ | 17 | 10 | Fever, nausea, vomiting, |
| | | | elevated liver enzymes, |
| | | | thrombocytopenia |
| 3.3 × 10⁹ | 1 | 1 | Nausea, vomiting, |

### Standard Operating Procedure for Vaccine Administration

A standard operating procedure was developed for the administration of ADXS31-164.

One hour prior to vaccination patients receive 2 mg/kg diphenhydramine via intramuscular injection and 0.2 mg/kg ondansetron as a slow intravenous push. The vaccine was kept at −80° C. and thawed patient-side. It was administered in 200 mls of 0.9% NaCl over 30 mins. The infusion line is then flushed with 30 mls of Plasmalyte. Dogs are sent home with a three day course of amoxicillin (to start 72 hours post vaccination) and a 7 day course of liver supplement (S-adenosyl-methionine) that aids in cellular growth and repair.

The primary endpoint of the study was to determine the maximum tolerated dose of ADXS31-164.

Doses up to 3.3×10⁹ were well tolerated in dogs ranging in body weight from 25 kg to 67 kg. All side effects reported were grade I toxicities and the maximum tolerated dose has yet to be reached. Side effects routinely occurred within 2-4 hours of vaccine administration. High fevers usually resolved with intravenous isotonic fluids delivered at maintenance rate (4 mls/kg/hour) for 2-4 hours. In two cases where fevers reached 104.7 and above, a single subcutaneous injection of carprofen induced normothermia within 1-2 hours. Nausea and vomiting was usually self-limiting but in cases where several episodes are noted, 1 mg/kg cerenia is administered and this was very effective at preventing further nausea and vomiting. A total of 5 dogs developed mild, grade I elevations in liver enzymes within 48 hours of vaccine administration—these resolved by one week post vaccination.

### Clearance of Listeria

After performing blood cultures on all 16 dogs vaccinated to date there was no detectable listeria in the peripheral circulation of any of the dogs at 24 hours post vaccination. Shedding of listeria in the urine and feces of vaccinated dogs was not assessed.

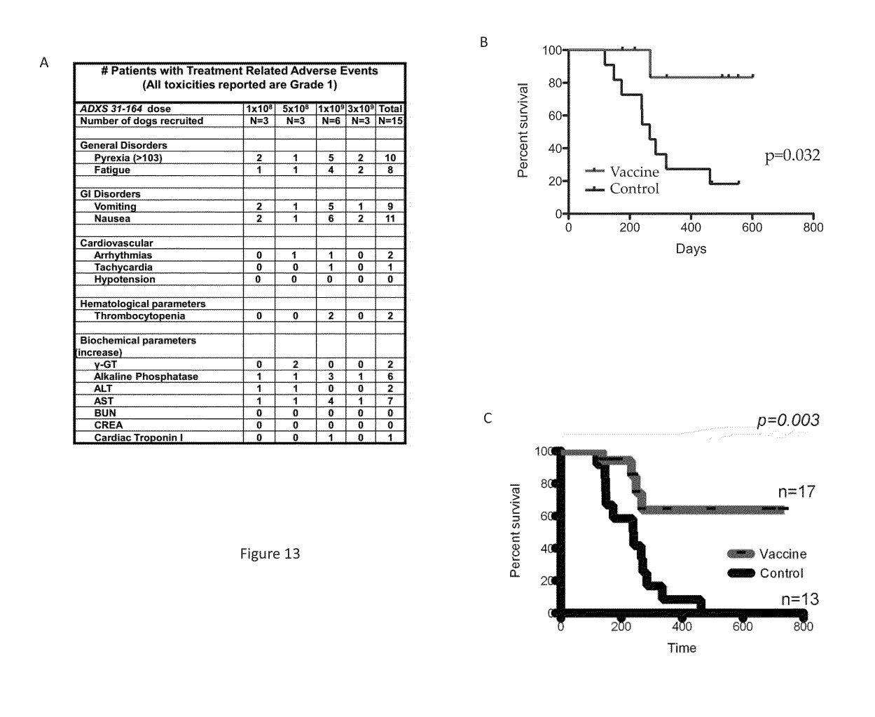

Secondary endpoints for the study are progression-free survival and overall survival. A statistically significant overall survival advantage in dogs with osteosarcoma has been observed when ADXS31-164 is administered after limb amputation and 4 doses of carboplatin. Early results from the first two dose groups (6 dogs) show a significant survival advantage in dogs that received ADXS31-164 compared to 6 dogs whose owners elected not to participate in the trial but who were followed for survival (p=0.003) (FIG. 13). The mean survival time for unvaccinated dogs is 239.5 days. The mean survival time for vaccinated dogs has not yet been reached. This remains true when all dogs within the intent to treat group are included in analysis.

In conclusion, there was no evidence of significant short or long-term side effects on the cardiovascular, hematopoietic, hepatic, or renal systems. Moreover, administration of ADXS31-164 in the presence of minimal residual disease can delay/prevent metastatic disease and prolong overall survival of dogs with Her2/neu positive osteosarcoma.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of treating a Her-2/neu-expressing tumor growth or cancer in a human subject, the method comprising the step of administering a recombinant attenuated Listeria comprising nucleic acid encoding a fusion polypeptide, wherein said fusion polypeptide comprises a Her2/neu chimeric antigen fused to an additional adjuvant polypeptide, wherein said nucleic acid molecule comprises a first open reading frame encoding said fusion polypeptide, wherein said nucleic acid molecule further comprises a second open reading frame encoding a metabolic enzyme, and wherein said metabolic enzyme complements an endogenous gene that is lacking in the chromosome of said recombinant Listeria vaccine strain.

2. The method of claim 1, wherein said human subject is a child, an adolescent or an adult.

3. The method of claim 1, wherein administering said fusion polypeptide to said subject prevents escape mutations within said tumor.

4. The method of claim 1, wherein said Her2/neu chimeric antigen comprises at least 5, 9, 13, 14, or 17 of the mapped human MHC-class I epitopes.

5. The method of claim 1, wherein said nucleic acid molecule is integrated into the Listeria genome.

6. The method of claim 1, wherein said nucleic acid molecule is in a plasmid in said recombinant Listeria vaccine strain and wherein said plasmid is stably maintained in said recombinant Listeria vaccine strain in the absence of antibiotic selection.

7. The method of claim 1, wherein said recombinant Listeria lacks the ActA virulence gene.

8. The method of claim 1, wherein said additional polypeptide is selected from the group consisting of: a) non-hemolytic LLO protein or N-terminal fragment, b) a PEST sequence, or c) an ActA fragment.

9. The method of claim 1, wherein said metabolic enzyme encoded by said second open reading frame is an alanine racemase enzyme or a D-amino acid transferase enzyme.

10. The method of claim 1, further comprising an independent adjuvant.

11. The method of claim 11, wherein said adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

12. The method of claim 1, wherein said tumor is a Her2/neu positive tumor and wherein said cancer is a Her2/neu-expressing cancer.

13. The method of claim 1, wherein said cancer is osteosarcoma or Ewing's sarcoma (ES).

14. A method of eliciting an enhanced immune response against a Her-2/neu-expressing tumor growth or cancer in a human subject, the method comprising the step of administering a recombinant attenuated Listeria comprising a nucleic acid encoding a fusion polypeptide, wherein said fusion polypeptide comprises a Her2/neu chimeric antigen fused to an additional adjuvant polypeptide, wherein said nucleic acid molecule comprises a first open reading frame encoding said fusion polypeptide, wherein said nucleic acid molecule further comprises a second open reading frame encoding a metabolic enzyme, and wherein said metabolic enzyme complements an endogenous gene that is lacking in the chromosome of said recombinant Listeria vaccine strain.

15. The method of claim 15, wherein said human subject is a child, an adolescent or an adult.

16. The method of claim 15, wherein administering said fusion polypeptide to said subject prevents escape mutations within said tumor.

17. The method of claim 15, wherein said Her2/neu chimeric antigen comprises at least 5, 9, 13, 14, or 17 of the mapped human MHC-class I epitopes.

18. The method of claim 15, wherein said nucleic acid molecule is integrated into the Listeria genome.

19. The method of claim 15, wherein said nucleic acid molecule is in a plasmid in said recombinant Listeria vaccine strain.

20. The method of claim 15, wherein said plasmid is stably maintained in said recombinant Listeria vaccine strain in the absence of antibiotic selection.

21. The method of claim 15, wherein said recombinant Listeria lacks the ActA virulence gene.

22. The method of claim 15, wherein said additional polypeptide is selected from the group consisting of: a) non-hemolytic LLO protein or N-terminal fragment, b) a PEST sequence, or c) an ActA fragment.

23. The method of claim 15, wherein said metabolic enzyme encoded by said second open reading frame is an alanine racemase enzyme or a D-amino acid transferase enzyme.

24. The method of claim 15, further comprising an independent adjuvant.

25. The method of claim 25, wherein said adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

26. The method of claim 15, wherein said tumor is a Her2/neu positive tumor and wherein said cancer is a Her2/neu-expressing cancer.

27. The method of claim 15, wherein said cancer is osteosarcoma or Ewing's sarcoma (ES).

28. The method of claim 16, wherein said immune response against said Her2/neu-expressing tumor or cancer comprises an immune response to a subdominant epitope of said Her2/neu protein.
